(19) United States
(12) Patent Application Publication
(10) Pub. No.: US 2003/0170694 A1
(43) Pub. Date: Sep. 11, 2003

(54) Stabilized nucleic acids in gene and drug discovery and methods of use

(76) Inventors: Daniel Wall, San Diego, CA (US); Jamie Froelich, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 10/327,592

(22) Filed: Dec. 20, 2002

Related U.S. Application Data
(60) Provisional application No. 60/343,512, filed on Dec. 21, 2001.

(51) Int. Cl. C12Q 1/68; C12Q 1/18

(52) U.S. Cl. 435/6; 435/32

(57) ABSTRACT

Stabilized nucleic acids for use in gene and drug discovery are disclosed. Vectors and host cells useful in the production of stabilized nucleic acids are also disclosed. Cell-based assays which employ stabilized antisense nucleic acids to identify and develop antibiotics and to identify genes required for proliferation are described. The use of stabilized nucleic acids to identify homologous nucleic acids required for the proliferation of heterologous organisms is also described. Inhibition of the expression of genes required for proliferation in heterologous organisms through the use of stabilized antisense nucleic acids is disclosed.

### RELATED APPLICATIONS

This application claims priority to U.S. Provisional Patent Application Serial No. 60/343,512, filed Dec. 21, 2001, by Daniel Wall, et al., and entitled “STABILIZED NUCLEIC ACIDS IN GENE AND DRUG DISCOVERY AND METHODS OF USE”, the disclosure of which is incorporated herein by reference in its entirety.

### BACKGROUND OF THE INVENTION

Since the discovery of penicillin, the use of antibiotics to treat the ravages of bacterial infections has saved millions of lives. With the advent of these “miracle drugs,” for a time it was popularly believed that humanity might, once and for all, be saved from the scourge of bacterial infections. In fact, during the 1980s and early 1990s, many large pharmaceutical companies cut back or eliminated antibiotics research and development. They believed that infectious disease caused by bacteria finally had been conquered and that markets for new drugs were limited. Unfortunately, this belief was overly optimistic.

The tide is beginning to turn in favor of the bacteria as reports of drug resistant bacteria become more frequent. The United States Centers for Disease Control announced that one of the most powerful known antibiotics, vancomycin, was unable to treat an infection of the common Staphylococcus aureus (staph). This organism is commonly found in our environment and is responsible for many nosocomial infections. The import of this announcement becomes clear when one considers that vancomycin was used for years to treat infections caused by Staphylococcus species as well as other stubborn strains of bacteria. In short, bacteria are becoming resistant to our most powerful antibiotics. If this trend continues, it is conceivable that we will return to a time when what are presently considered minor bacterial infections are fatal diseases.

Over-prescription and improper prescription habits by some physicians have caused an indiscriminate increase in the availability of antibiotics to the public. The patients are also partly responsible, since they will often improperly use the drug, thereby generating yet another population of bacteria that is resistant, in whole or in part, to traditional antibiotics.

The bacterial pathogens that have haunted humanity remain, in spite of the development of modern scientific practices to deal with the diseases that they cause. Drug resistant bacteria are now an increasing threat to the health of humanity. A new generation of antibiotics is needed to once again deal with the pending health threat that bacteria present.

### Discovery of New Antibiotics

As more and more bacterial strains become resistant to the panel of available antibiotics, new antibiotics are required to treat infections. In the past, practitioners of pharmacology would have to rely upon traditional methods of drug discovery to generate novel, safe and efficacious compounds for the treatment of disease. Traditional drug discovery methods involve blindly testing potential drug candidate-molecules, often selected at random, in the hope that one might prove to be an effective treatment for some disease. The process is painstaking and laborious, with no guarantee of success. Today, the average cost to discover and develop a new drug exceeds US $500 million, and the average time from laboratory to patient is 15 years. Improving this process, even incrementally, would represent a huge advance in the generation of novel antimicrobial agents.

Newly emerging practices in drug discovery utilize a number of biochemical techniques to provide for directed approaches to creating new drugs, rather than discovering them at random. For example, gene sequences and proteins encoded thereby that are required for the proliferation of a cell or microorganism make excellent targets since exposure of bacteria to compounds active against these targets would result in the inactivation of the cell or microorganism. Once a target is identified, biochemical analysis of that target can be used to discover or to design molecules that interact with and alter the functions of the target. Use of physical and computational techniques to analyze structural and biochemical properties of targets in order to derive compounds that interact with such targets is called rational drug design and offers great potential. Thus, emerging drug discovery practices use molecular modeling techniques, combinatorial chemistry approaches, and other means to produce and screen and/or design large numbers of candidate compounds.

Nevertheless, while this approach to drug discovery is clearly the way of the future, problems remain. For example, the initial step of identifying molecular targets for investigation can be an extremely time consuming task. It may also be difficult to design molecules that interact with the target by using computer modeling techniques. Furthermore, in cases where the function of the target is not known or is poorly understood, it may be difficult to design assays to detect molecules that interact with and alter the functions of the target. To improve the rate of novel drug discovery and development, methods of identifying important molecular targets in pathogenic cells or microorganisms and methods for identifying molecules that interact with and alter the functions of such molecular targets are urgently required.

Staphylococcus aureus is a Gram positive microorganism which is the causative agent of many infectious diseases. Local infection by Staphylococcus aureus can cause abscesses on skin and cellulitis in subcutaneous tissues and can lead to toxin-related diseases such as toxic shock and scalded skin syndromes. Staphylococcus aureus can cause serious systemic infections such as osteomyelitis, endocarditis, pneumonia, and septicemia. Staphylococcus aureus is also a common cause of food poisoning, often arising from contact between prepared food and infected food industry workers. Antibiotic resistant strains of Staphylococcus aureus have recently been identified, including those that are now resistant to all available antibiotics, thereby severely limiting the options of care available to physicians.

Pseudomonas aeruginosa is an important Gram-negative opportunistic pathogen. It is the most common Gram-negative found in nosocomial infections. P. aeruginosa is responsible for 16% of nosocomial pneumonia cases, 12% of hospital-acquired urinary tract infections, 8% of surgical wound infections, and 10% of bloodstream infections. Immunocompromised patients, such as neutropenic cancer and bone marrow transplant patients, are particular susceptible to opportunistic infections. In this group of patients, P. aeruginosa is responsible for pneumonia and septicemia with attributable deaths reaching 30%. P. aeruginosa is also one of the most common and lethal pathogens responsible for ventilator-associated pneumonia in intubated patients, with directly attributable death rates reaching 38%. Although P. aeruginosa outbreaks in burn patients are rare, it is associated with 60% death rates. In the AIDS population, P. aeruginosa is associated with 50% of deaths. Cystic fibrosis patients are characteristically susceptible to chronic infection by P. aeruginosa, which is responsible for high rates of illness and death. Current antibiotics work poorly for CF infections (Van Delden & Igelwski. 1998. Emerging Infectious Diseases 4:551-560; references therein).

The gram-negative enteric bacterial genus, Salmonella, encompasses at least 2 species. One of these, S. enterica, is divided into multiple subspecies and thousands of serotypes or serovars (Brenner, et al. 2000 J. Clin. Microbiol. 38:2465-2467). The S. enterica human pathogens include serovars Typhi, Paratyphi, Typhimurium, Cholerasuis, and many others deemed so closely related that they are variants of a widespread species. Worldwide, disease in humans caused by Salmonella is a very serious problem. In many developing countries, S. enterica ser. Typhi still causes often-fatal typhoid fever. This problem has been reduced or eliminated in wealthy industrial states. However, enteritis induced by Salmonella is widespread and is the second most common disease caused by contaminated food in the United States (Edwards, B H 1999 “Salmonella and Shigella species” Clin. Lab Med. 19(3):469-487). Though usually self-limiting in healthy individuals, others such as children, seniors, and those with compromising illnesses can be at much greater risk of serious illness and death.

Some S. enterica serovars (e.g. Typhimurium) cause a localized infection in the gastrointestinal tract. Other serovars (i.e. Typhi and Paratyphi) cause a much more serious systemic infection. In animal models, these roles can be reversed which has allowed the use of the relatively safe S. enterica ser. Typhimurium as a surrogate in mice for the typhoid fever agent, S. enterica ser. Typhi. In mice, S. enterica ser Typhimurium causes a systemic infection similar in outcome to typhoid fever. Years of study of the Salmonella have led to the identification of many determinants of virulence in animals and humans. Salmonella is interesting in its ability to localize to and invade the intestinal epithelium, induce morphologic changes in target cells via injection of certain cell-remodeling proteins, and to reside intracellularly in membrane-bound vesicles (Wallis, T S and Galyov, E E 2000 “Molecular basis of Salmonella-induced enteritis.” Molec. Microb. 36:997-1005; Falkow, S “The evolution of pathogenicity in Escherichia, Shigella, and Salmonella,” Chap. 149 in Neidhardt, et al. eds pp 2723-2729; Gulig, P A “Pathogenesis of Systemic Disease,” Chap. 152 in Neidhardt, et al. ppp 2774-2787). The immediate infection often results in a severe watery diarrhea but Salmonella also can establish and maintain a subclinical carrier state in some individuals. Spread is via food contaminated with sewage.

The gene products implicated in Salmonella pathogenesis include type three secretion systems (TTSS), proteins affecting cytoplasmic structure of the target cells, many proteins carrying out functions necessary for survival and proliferation of Salmonella in the host, as well as “traditional” factors such as endotoxin and secreted exotoxins. Additionally, there must be factors mediating species-specific illnesses. Despite this most of the genomes of S. enterica ser. Typhi (see http://www.sanger.ac.uk/Projects/S_typhi/ for the genome database) and S. enterica ser. Typhimurium (see http://genome.wustl.edu/gsc/bacterial/salmonella.shtml for the genome database) are highly conserved and are mutually useful for gene identification in multiple serovars. The Salmonella are a complex group of enteric bacteria causing disease similar to but distinct from other gram-negative enterics such as E. coli and have been a focus of biomedical research for the last century.

Enterococcus faecalis, a Gram-positive bacterium, is by far the most common member of the enterococci to cause infections in humans. Enterococcus faecium generally accounts for less than 20% of clinical isolates. Enterococci infections are mostly hospital-acquired though they are also associated with some community-acquired infections. Of nosocomial infections enterococci account for 12% of bacteremia, 15% of surgical wound infections, 14% of urinary tract infections, and 5 to 5% of endocarditis cases (Huycke, M. M., D. F., Sahm and M. S. Gilmore. 1998. Emerging Infectious Diseases 4:239-249). Additionally enterococci are frequently associated with intraabdominal and pelvic infections. Enterococci infections are often hard to treat because they are resistant to a vast array of antimicrobial drugs, including aminoglycosides, penicillin, ampicillin and vancomycin. The development of multiple-drug resistant (MDR) enterococci has made this bacteria a major concern for treating nosocomial infections.

These are just a few reasons which underscore the urgency of developing new antibiotics that are effective against pathogenic microorganisms. Accordingly, there is a need to refine the methods used to identify and characterize bacterial genomic sequences that encode gene products involved in proliferation, which can be used to identify potential new targets for antibiotic development. Prior to antisense based gene and drug discovery methods, such as those described in U.S. Pat. No. 6,228,579 and International Publication WO 01/70955, the discovery of genes required for the proliferation of pathogenic microorganism was a painstaking and slow process. The antisense approaches described in the above publications have been successfully used to expedite both the identification of proliferation-required genes in various organisms and the discovery of novel compounds that detrimentally effect organisms having decreased expression of those genes.

Antisene molecules, which provide the foundation for the above gene and drug discovery approaches, are introduced into the host organisms through the use of expression vectors. Methods that rely such vectors to introduce proliferation-inhibiting antisense transcripts into host organisms, however, are limited by the stability of these expressed RNA transcripts. Because certain RNA molecules are subject to rapid degradation inside the host organism, the effective concentration of such RNA molecules, and thus their inhibitory effect, is significantly reduced. Accordingly, proliferation-required genes that are complementary to such antisense molecules may escape detection. In order to increase the sensitivity and overall effectiveness of these important antisense-based gene and drug discovery approaches, there is a need to develop methods of providing host organisms with proliferation-inhibiting antisense transcripts that are resistance to degradation

### SUMMARY OF THE INVENTION

Some aspects of the present invention are described in the numbered paragraphs below.

1. A method for identifying genes involved in microbial proliferation comprising:

constructing a nucleotide sequence comprising an antisense nucleic acid flanked on each end by at least one stem-loop structure;

introducing the nucleotide sequence into a microorganism such that the antisense nucleic acid is present in the microorganism;

identifying an antisense nucleic acid which inhibits the proliferation of the microorganism; and

identifying the gene to which at least a portion of the identified antisense nucleic acid is complementary.

2. The method of Paragraph 1, wherein the at least one stem-loop structure formed at the 5′ end of the antisense nucleic acid comprises a flush, double stranded 5′ end.

3. The method of Paragraph 1, wherein the activity of at least one enzyme involved in RNA degradation has been reduced in the microorganism.

4. The method of Paragraph 3, wherein the at least one enzyme involved in RNA degradation is selected from the group consisting of RNase E, RNase II, RNase III, polynucleotide phosphorylase, and poly(A) polymerase.

5. The method of Paragraph 1, wherein the microorganism has a reduced activity of at least one RNA helicase.

6. The method of Paragraph 1, wherein the microorganism has a reduced activity of enolase.

7. The method of Paragraph 1, wherein the antisense nucleic acid comprises a random genomic fragment from the microorganism.

8. The method of Paragraph 7, wherein the step of introducing the nucleotide sequence comprises transcribing the nucleotide sequence from a promoter.

9. The method of Paragraph 8, wherein the first transcribed nucleotide from the promoter is the first nucleotide of a 5′ stem-loop structure.

10. The method of Paragraph 8, wherein the promoter is regulatable.

11. The method of Paragraph 10, wherein the promoter is inducible.

12. The method of Paragraph 11, wherein the step of identifying the antisense nucleic acid which inhibits the proliferation of the microorganism comprises comparing the proliferation of the microorganism transcribing a first level of the nucleotide sequence to the proliferation of the microorganism which transcribes a lower level of the nucleotide sequence or which does not transcribe the nucleotide sequence.

13. The method of Paragraph 1, wherein the nucleotide sequence is RNA.

14. The method of Paragraph 13, wherein the RNA is untranslated.

15. The method of Paragraph 1, wherein the microorganism is a gram-negative bacterium.

16. The method of Paragraph 1, wherein one the stem-loop structure comprises SEQ ID NO.: 5.

17. The method of Paragraph 1, wherein the free energy of formation is less than or equal to −7 kcal/mol.

18. The method of Paragraph 1, wherein the stem of the at least one stem-loop structure comprises at least eight base pairs.

19. The method of Paragraph 1, wherein the stem of the at least one stem-loop structure comprises at least twenty-five percent GC base pairs.

20. The method of Paragraph 1, wherein the loop of the at least one stem-loop structure comprises at least five nucleotides.

21. The method of Paragraph 1, wherein the nucleotide sequence lacks RNase E recognition sites.

22. The method of Paragraph 1, wherein the at least one stem-loop structure lacks RNase III recognition sites.

23. The method of Paragraph 1, wherein the at least one stem-loop structure lacks a ribosome binding site.

24. The method of Paragraph 1, wherein the at least one stem-loop structure formed at the 3′ end of the antisense nucleic acid comprises at least one rho independent terminator.

25. The method of Paragraph 24, wherein the at least one rho independent terminator comprises rrnBt1 and rrnBt2.

26. The method of Paragraph 1, wherein the microorganism is selected from a group consisting of Bacteroides fragilis, Bordetella pertussis, Burkholderia cepacia, Campylobacter jejuni, Chlamydia pneumoniae, Chlamydia trachomatus, Enterobacter cloacae, Escherichia coli, Haemophilus influenzae, Helicobacter pylori, Klebsiella pneumoniae, Neisseria gonorrhoeae, Neisseria meningitidis, Pasteurella haemolytica, Pasteurella multocida, Proteus vulgaris, Pseudomonas aeruginosa, Salmonella bongori, Salmonella cholerasuis, Salmonella enterica, Salmonella paratyphi, Salmonella typhi, Salmonella typhimurium, Moxarella catarrhalis, Shigella boydii, Shigella dysenteriae, Shigella flexneri, Shigella sonnei, Treponema pallidum, Yersinia enterocolitica, Yersinia pestis or any species falling within the genera of any of the above species.

27. The method of Paragraph 1, wherein the microorganism is selected from a group consisting of Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species.

28. A method for identifying a compound which reduces the activity or level of a gene product required for proliferation of a cell comprising the steps of:

(a) sensitizing a cell by providing a sublethal level of an antisense nucleic acid comprising a nucleotide sequence complementary to at least a portion of the gene encoding the gene product in the cell, wherein the antisense nucleic acid is flanked on each end by at least one stem-loop structure;

(b) contacting the sensitized cell with a compound; and

(c) determining the degree to which the compound inhibits proliferation of the sensitized cell relative to a cell which has not been sensitized.

29. The method of Paragraph 28, wherein the at least one stem-loop structure formed at the 5′ end of the antisense nucleic acid comprises a flush, double stranded 5′ end.

30. The method of Paragraph 28, wherein the activity of at least one enzyme involved in RNA degradation has been reduced in the sensitized cell.

31. The method of Paragraph 30, wherein the at least one enzyme involved in RNA degradation is selected from the group consisting of RNase E, RNase II, RNase III, polynucleotide phosphorylase, and poly(A) polymerase.

32. The method of Paragraph 28, wherein the sensitized cell has a reduced activity of at least one RNA helicase.

33. The method of Paragraph 28, wherein the sensitized cell has a reduced activity of enolase.

34. The method of Paragraph 28, wherein the step of sensitizing the cell comprises transcribing the antisense nucleic acid from a promoter.

35. The method of Paragraph 34, wherein the first transcribed nucleotide from the promoter is the first nucleotide of a 5′ stem-loop structure.

36. The method of Paragraph 34, wherein the promoter is regulatable.

37. The method of Paragraph 36, wherein the promoter is inducible.

38. The method of Paragraph 28, wherein the antisense nucleic acid is RNA.

39. The method of Paragraph 38, wherein the RNA is untranslated.

40. The method of Paragraph 28, wherein the sensitized cell is a gram-negative bacterium.

41. The method of Paragraph 28, wherein one the stem-loop structure comprises SEQ ID NO.: 5.

42. The method of Paragraph 28, wherein the free energy of formation is less than or equal to −7 kcal/mol.

43. The method of Paragraph 28, wherein the stem of the at least one stem-loop structure comprises at least eight base pairs.

44. The method of Paragraph 28, wherein the stem of the at least one stem-loop structure comprises at least twenty-five percent GC base pairs.

45. The method of Paragraph 28, wherein the loop of the at least one stem-loop structure comprises at least five nucleotides.

46. The method of Paragraph 28, wherein the antisense nucleic acid lacks RNase E recognition sites.

47. The method of Paragraph 28, wherein the at least one stem-loop structure lacks RNase III recognition sites.

48. The method of Paragraph 28, wherein the at least one stem-loop structure lacks a ribosome binding site.

49. The method of Paragraph 28, wherein the at least one stem-loop structure formed at the 3′ end of the antisense nucleic acid comprises at least one rho independent terminator.

50. The method of Paragraph 49, wherein the at least one rho independent terminator comprises rrnBt1 and rrnBt2.

51. The method of Paragraph 28, wherein the gene product is an RNA.

52. The method of Paragraph 28, wherein the gene product is a polypeptide.

53. The method of Paragraph 28, wherein the sensitized cell is selected from a group consisting of Bacteroides fragilis, Bordetella pertussis, Burkholderia cepacia, Campylobacter jejuni, Chlamydia pneumoniae, Chlamydia trachomatus, Enterobacter cloacae, Escherichia coli, Haemophilus influenzae, Helicobacter pylori, Klebsiella pneumoniae, Neisseria gonorrhoeae, Neisseria meningitidis, Pasteurella haemolytica, Pasteurella multocida, Proteus vulgaris, Pseudomonas aeruginosa, Salmonella bongori, Salmonella cholerasuis, Salmonella enterica, Salmonella paratyphi, Salmonella typhi, Salmonella typhimurium, Moxarella catarrhalis, Shigella boydii, Shigella dysenteriae, Shigella flexneri, Shigella sonnei, Treponema pallidum, Yersinia enterocolitica, Yersinia pestis or any species falling within the genera of any of the above species.

54. The method of Paragraph 28, wherein the sensitized cell is selected from a group consisting of Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species.

55. A compound identified using the method of Paragraph 28.

56. A method for inhibiting the activity or expression of a gene in an operon required for proliferation comprising contacting a cell in a cell population with a nucleotide sequence comprising an antisense nucleic acid flanked on each end by at least one stem-loop structure, wherein the antisense nucleic acid is complementary to at least a portion of the operon.

57. The method of Paragraph 56, wherein the at least one stem-loop structure formed at the 5′ end of the antisense nucleic acid comprises a flush, double stranded 5′ end.

58. The method of Paragraph 56, wherein the activity of at least one enzyme involved in RNA degradation has been reduced in the cell.

59. The method of Paragraph 58, wherein the at least one enzyme involved in RNA degradation is selected from the group consisting of RNase E, RNase II, RNase III, polynucleotide phosphorylase, and poly(A) polymerase.

60. The method of Paragraph 56, wherein the cell has a reduced activity of at least one RNA helicase.

61. The method of Paragraph 56, wherein the cell has a reduced activity of enolase.

62. The method of Paragraph 56, wherein the step of contacting the cell in the cell population with the nucleotide sequence comprises transcribing the nucleotide sequence from a promoter.

63. The method of Paragraph 62, wherein the first transcribed nucleotide from the promoter is the first nucleotide of a 5′ stem-loop structure.

64. The method of Paragraph 62, wherein the promoter is regulatable.

65. The method of Paragraph 64, wherein the promoter is inducible.

66. The method of Paragraph 56, wherein the nucleotide sequence is RNA.

67. The method of Paragraph 66, wherein the RNA is untranslated.

68. The method of Paragraph 56, wherein the cell is a gram-negative bacterium.

69. The method of Paragraph 56, wherein one the stem-loop structure comprises SEQ ID NO.: 5.

70. The method of Paragraph 56, wherein the free energy of formation is less than or equal to −7 kcal/mol.

71. The method of Paragraph 56, wherein the stem of the at least one stem-loop structure comprises at least eight base pairs.

72. The method of Paragraph 56, wherein the stem of the at least one stem-loop structure comprises at least twenty-five percent GC base pairs.

73. The method of Paragraph 56, wherein the loop of the at least one stem-loop structure comprises at least five nucleotides.

74. The method of Paragraph 56, wherein the nucleotide sequence lacks RNase E recognition sites.

75. The method of Paragraph 56, wherein the at least one stem-loop structure lacks RNase III recognition sites.

76. The method of Paragraph 56, wherein the at least one stem-loop structure lacks a ribosome binding site.

77. The method of Paragraph 56, wherein the at least one stem-loop structure formed at the 3′ end of the antisense nucleic acid comprises at least one rho independent terminator.

78. The method of Paragraph 77, wherein the at least one rho independent terminator comprises rrnBt1 and rrnBt2.

79. The method of Paragraph 56, wherein the cell is selected from a group consisting of Bacteroides fragilis, Bordetella pertussis, Burkholderia cepacia, Campylobacter jejuni, Chlamydia pneumoniae, Chlamydia trachomatus, Enterobacter cloacae, Escherichia coli, Haemophilus influenzae, Helicobacter pylori, Klebsiella pneumoniae, Neisseria gonorrhoeae, Neisseria meningitidis, Pasteurella haemolytica, Pasteurella multocida, Proteus vulgaris, Pseudomonas aeruginosa, Salmonella bongori, Salmonella cholerasuis, Salmonella enterica, Salmonella paratyphi, Salmonella typhi, Salmonella typhimurium, Moxarella catarrhalis, Shigella boydii, Shigella dysenteriae, Shigella flexneri, Shigella sonnei, Treponema pallidum, Yersinia enterocolitica, Yersinia pestis or any species falling within the genera of any of the above species.

80. The method of Paragraph 56, wherein the cell is selected from a group consisting of Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species.

81. A method for identifying a gene which is required for proliferation of a cell comprising:

(a) contacting a cell with a nucleotide sequence comprising an antisense nucleic acid flanked on each end by at least one stem-loop structure, wherein the cell is a cell other than the organism from which the antisense nucleic acid was obtained;

(b) determining whether the nucleotide sequence inhibits proliferation of the cell; and

(c) identifying the gene in the cell which encodes the mRNA which is complementary to the antisense polynucleotide region or a portion thereof.

82. The method of Paragraph 81, wherein the at least one stem-loop structure formed at the 5′ end of the antisense nucleic acid comprises a flush, double stranded 5′ end.

83. The method of Paragraph 81, wherein the activity of at least one enzyme involved in RNA degradation has been reduced in the cell.

84. The method of Paragraph 83, wherein the at least one enzyme involved in RNA degradation is selected from the group consisting of RNase E, RNase II, RNase III, polynucleotide phosphorylase, and poly(A) polymerase.

85. The method of Paragraph 81, wherein the cell has a reduced activity of at least one RNA helicase.

86. The method of Paragraph 81, wherein the cell has a reduced activity of enolase.

87. The method of Paragraph 81, wherein the antisense nucleic acid comprises a random genomic fragment from the organism.

88. The method of Paragraph 87, wherein the step of contacting the cell with the nucleotide sequence comprises transcribing the nucleotide sequence from a promoter.

89. The method of Paragraph 88, wherein the first transcribed nucleotide from the promoter is the first nucleotide of a 5′ stem-loop structure.

90. The method of Paragraph 88, wherein the promoter is regulatable.

91. The method of Paragraph 90, wherein the promoter is inducible.

92. The method of Paragraph 91, wherein the step of determining whether the nucleotide sequence inhibits the proliferation of the cell comprises comparing the proliferation of the cell transcribing a first level of the nucleotide sequence to the proliferation of the cell which transcribes a lower level of the nucleotide sequence or which does not transcribe the nucleotide sequence.

93. The method of Paragraph 81, wherein the nucleotide sequence is RNA.

94. The method of Paragraph 93, wherein the RNA is untranslated.

95. The method of Paragraph 81, wherein the cell is a gram-negative bacterium.

96. The method of Paragraph 81, wherein one the stem-loop structure comprises SEQ ID NO.: 5.

97. The method of Paragraph 81, wherein the free energy of formation is less than or equal to −7 kcal/mol.

98. The method of Paragraph 81, wherein the stem of the at least one stem-loop structure comprises at least eight base pairs.

99. The method of Paragraph 81, wherein the stem of the at least one stem-loop structure comprises at least twenty-five percent GC base pairs.

100. The method of Paragraph 81, wherein the loop of the at least one stem-loop structure comprises at least five nucleotides.

101. The method of Paragraph 81, wherein the nucleotide sequence lacks RNase E recognition sites.

102. The method of Paragraph 81, wherein the at least one stem-loop structure lacks RNase III recognition sites.

103. The method of Paragraph 81, wherein the at least one stem-loop structure lacks a ribosome binding site.

104. The method of Paragraph 81, wherein the at least one stem-loop structure formed at the 3′ end of the antisense nucleic acid comprises at least one rho independent terminator.

105. The method of Paragraph 104, wherein the at least one rho independent terminator comprises rrnBt1 and rrnBt2.

106. The method of Paragraph 81, wherein the cell is selected from a group consisting of Bacteroides fragilis, Bordetella pertussis, Burkholderia cepacia, Campylobacter jejuni, Chlamydia pneumoniae, Chlamydia trachomatus, Enterobacter cloacae, Escherichia coli, Haemophilus influenzae, Helicobacter pylori, Klebsiella pneumoniae, Neisseria gonorrhoeae, Neisseria meningitidis, Pasteurella haemolytica, Pasteurella multocida, Proteus vulgaris, Pseudomonas aeruginosa, Salmonella bongori, Salmonella cholerasuis, Salmonella enterica, Salmonella paratyphi, Salmonella typhi, Salmonella typhimurium, Moxarella catarrhalis, Shigella boydii, Shigella dysenteriae, Shigella flexneri, Shigella sonnei, Treponema pallidum, Yersinia enterocolitica, Yersinia pestis or any species falling within the genera of any of the above species.

107. The method of Paragraph 81, wherein the cell is selected from a group consisting of Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species.

108. A method for identifying the biological pathway in which a proliferation-required gene or its gene product lies comprising:

(a) providing a sublethal level of a nucleotide sequence comprising an antisense nucleic acid complementary to at least a portion of the gene encoding the gene product to a test cell, wherein the antisense nucleic acid is flanked on each end by at least one stem-loop structure and wherein the nucleotide sequence inhibits the activity of the proliferation-required gene or gene product in the test cell;

(b) contacting the test cell with a compound known to inhibit growth or proliferation of a cell, wherein the biological pathway on which the compound acts is known; and

(c) determining the degree to which the proliferation of the test cell is inhibited relative to a cell which was not contacted with the compound.

109. The method of Paragraph 108, wherein the at least one stem-loop structure formed at the 5′ end of the antisense nucleic acid comprises a flush, double stranded 5′ end.

110. The method of Paragraph 108, wherein the activity of at least one enzyme involved in RNA degradation has been reduced in the test cell.

111. The method of Paragraph 110, wherein the at least one enzyme involved in RNA degradation is selected from the group consisting of RNase E, RNase II, RNase III, polynucleotide phosphorylase, and poly(A) polymerase.

112. The method of Paragraph 108, wherein the test cell has a reduced activity of at least one RNA helicase.

113. The method of Paragraph 108, wherein the test cell has a reduced activity of enolase.

114. The method of Paragraph 108, wherein the step of providing the sublethal level of the nucleotide sequence comprises transcribing the nucleotide sequence from a promoter.

115. The method of Paragraph 114, wherein the first transcribed nucleotide from the promoter is the first nucleotide of a 5′ stem-loop structure.

116. The method of Paragraph 114, wherein the promoter is regulatable.

117. The method of Paragraph 116, wherein the promoter is inducible.

118. The method of Paragraph 108, wherein the nucleotide sequence is RNA.

119. The method of Paragraph 118, wherein the RNA is untranslated.

120. The method of Paragraph 108, wherein the test cell is a gram-negative bacterium.

121. The method of Paragraph 108, wherein one the stem-loop structure comprises SEQ ID NO.: 5.

122. The method of Paragraph 108, wherein the free energy of formation is less than or equal to −7 kcal/mol.

123. The method of Paragraph 108, wherein the stem of the at least one stem-loop structure comprises at least eight base pairs.

124. The method of Paragraph 108, wherein the stem of the at least one stem-loop structure comprises at least twenty-five percent GC base pairs.

125. The method of Paragraph 108, wherein the loop of the at least one stem-loop structure comprises at least five nucleotides.

126. The method of Paragraph 108, wherein the nucleotide sequence lacks RNase E recognition sites.

127. The method of Paragraph 108, wherein the at least one stem-loop structure lacks RNase III recognition sites.

128. The method of Paragraph 108, wherein the at least one stem-loop structure lacks a ribosome binding site.

129. The method of Paragraph 108, wherein the at least one stem-loop structure formed at the 3′ end of the antisense nucleic acid comprises at least one rho independent terminator.

130. The method of Paragraph 129, wherein the at least one rho independent terminator comprises rrnBt1 and rrnBt2.

131. The method of Paragraph 108,wherein the determining step comprises determining whether the test cell has substantially greater sensitivity to the compound than a cell which does not express the sublethal level of the nucleotide sequence.

132. The method of Paragraph 108, wherein the test cell is selected from a group consisting of Bacteroides fragilis, Bordetella pertussis, Burkholderia cepacia, Campylobacter jejuni, Chlamydia pneumoniae, Chlamydia trachomatus, Enterobacter cloacae, Escherichia coli, Haemophilus influenzae, Helicobacter pylori, Klebsiella pneumoniae, Neisseria gonorrhoeae, Neisseria meningitidis, Pasteurella haemolytica, Pasteurella multocida, Proteus vulgaris, Pseudomonas aeruginosa, Salmonella bongori, Salmonella cholerasuis, Salmonella enterica, Salmonella paratyphi, Salmonella typhi, Salmonella typhimurium, Moxarella catarrhalis, Shigella boydii, Shigella dysenteriae, Shigella flexneri, Shigella sonnei, Treponema pallidum, Yersinia enterocolitica, Yersinia pestis or any species falling within the genera of any of the above species.

133. The method of Paragraph 108, wherein the test cell is selected from a group consisting of Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species.

134. A method for determining the biological pathway on which a test compound acts comprising:

(a) by providing a sublethal level of a nucleotide sequence comprising an antisense nucleic acid complementary to at least a portion of a gene encoding a gene product required for proliferation in a first cell, wherein the antisense nucleic acid is flanked on each end by at least one stem-loop structure and wherein the antisense nucleic acid inhibits the activity or expression of the gene and wherein the biological pathway in which the gene or product of the gene lies is known,

(b) contacting the first cell with the test compound; and

(c) determining the degree to which the test compound inhibits proliferation of the first cell relative to a cell which does not contain the nucleotide sequence.

135. The method of Paragraph 134, wherein the at least one stem-loop structure formed at the 5′ end of the antisense nucleic acid comprises a flush, double stranded 5′ end.

136. The method of Paragraph 134, wherein the activity of at least one enzyme involved in RNA degradation has been reduced in the sensitized cell.

137. The method of Paragraph 136, wherein the at least one enzyme involved in RNA degradation is selected from the group consisting of RNase E, RNase II, RNase III, polynucleotide phosphorylase, and poly(A) polymerase.

138. The method of Paragraph 134, wherein the first cell has a reduced activity of at least one RNA helicase.

139. The method of Paragraph 134, wherein the first cell has a reduced activity of enolase.

140. The method of Paragraph 134, wherein the step of providing the sublethal level of the nucleotide sequence comprises transcribing the nucleotide sequence from a promoter.

141. The method of Paragraph 140, wherein the first transcribed nucleotide from the promoter is the first nucleotide of a 5′ stem-loop structure.

142. The method of Paragraph 140, wherein the promoter is regulatable.

143. The method of Paragraph 142, wherein the promoter is inducible.

144. The method of Paragraph 134, wherein the nucleotide sequence is RNA.

145. The method of Paragraph 144, wherein the RNA is untranslated.

146. The method of Paragraph 134, wherein the first cell is a gram-negative bacterium.

147. The method of Paragraph 134, wherein one the stem-loop structure comprises SEQ ID NO.: 5.

148. The method of Paragraph 134, wherein the free energy of formation is less than or equal to −7 kcal/mol.

149. The method of Paragraph 134, wherein the stem of the at least one stem-loop structure comprises at least eight base pairs.

150. The method of Paragraph 134, wherein the stem of the at least one stem-loop structure comprises at least twenty-five percent GC base pairs.

151. The method of Paragraph 134, wherein the loop of the at least one stem-loop structure comprises at least five nucleotides.

152. The method of Paragraph 134, wherein the nucleotide sequence lacks RNase E recognition sites.

153. The method of Paragraph 134, wherein the at least one stem-loop structure lacks RNase III recognition sites.

154. The method of Paragraph 134, wherein the at least one stem-loop structure lacks a ribosome binding site.

155. The method of Paragraph 134, wherein the at least one stem-loop structure formed at the 3′ end of the antisense nucleic acid comprises at least one rho independent terminator.

156. The method of Paragraph 155, wherein the at least one rho independent terminator comprises rrnBt1 and rrnBt2.

157. The method of Paragraph 134, wherein the determining step comprises determining whether the first cell has a substantially greater sensitivity to the test compound than a cell which does not express the sublethal level of the nucleotide sequence 158. The method of Paragraph 134, further comprising:

(d) providing a sublethal level of a second nucleotide sequence comprising an antisense nucleic acid complementary to at least a portion of a second gene encoding a gene product required for proliferation in a second cell, wherein the second gene encoding a gene product required for proliferation is in a different biological pathway than the gene encoding a gene product required for proliferation in step (a); and

(e) determining whether the second cell does not have a substantially greater sensitivity to the test compound than a cell which does not express the sublethal level of the second gene encoding a gene product required for proliferation, wherein the test compound is specific for the biological pathway against which the nucleotide sequence of step (a) acts if the first cell has a substantially greater sensitivity to the test compound than the second cell.

159. The method of Paragraph 134, wherein the sensitized cell is selected from a group consisting of Bacteroides fragilis, Bordetella pertussis, Burkholderia cepacia, Campylobacter jejuni, Chlamydia pneumoniae, Chlamydia trachomatus, Enterobacter cloacae, Escherichia coli, Haemophilus influenzae, Helicobacter pylori, Klebsiella pneumoniae, Neisseria gonorrhoeae, Neisseria meningitidis, Pasteurella haemolytica, Pasteurella multocida, Proteus vulgaris, Pseudomonas aeruginosa, Salmonella bongori, Salmonella cholerasuis, Salmonella enterica, Salmonella paratyphi, Salmonella typhi, Salmonella typhimurium, Moxarella catarrhalis, Shigella boydii, Shigella dysenteriae, Shigella flexneri, Shigella sonnei, Treponema pallidum, Yersinia enterocolitica, Yersinia pestis or any species falling within the genera of any of the above species.

160. The method of Paragraph 134, wherein the sensitized cell is selected from a group consisting of Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species.

161. A method for manufacturing an antibiotic comprising the steps of:

(a) contacting sensitized cells which express a sublethal level of a nucleotide sequence comprising an antisense nucleic acid flanked on each end by at least one stem-loop structure with a compound;

(b) identifying a compound which substantially inhibits the proliferation of the sensitized cells relative to cells which have not been sensitized; and

(c) manufacturing the compound so identified.

162. The method of Paragraph 161, wherein the at least one stem-loop structure formed at the 5′ end of the antisense nucleic acid comprises a flush, double stranded 5′ end.

163. The method of Paragraph 161, wherein the activity of at least one enzyme involved in RNA degradation has been reduced in the sensitized cells.

164. The method of Paragraph 163, wherein the at least one enzyme involved in RNA degradation is selected from the group consisting of RNase E, RNase II, RNase III, polynucleotide phosphorylase, and poly(A) polymerase.

165. The method of Paragraph 161, wherein the sensitized cells have a reduced activity of at least one RNA helicase.

166. The method of Paragraph 161, wherein the sensitized cells have a reduced activity of enolase.

167. The method of Paragraph 161, wherein the antisense nucleic acid comprises a random genomic fragment from the sensitized cells.

168. The method of Paragraph 161, wherein the nucleotide sequence is RNA.

169. The method of Paragraph 168, wherein the RNA is untranslated.

170. The method of Paragraph 161, wherein the sensitized cells comprise a gram-negative bacterium.

171. The method of Paragraph 161, wherein one the stem-loop structure comprises SEQ ID NO.: 5.

172. The method of Paragraph 161, wherein the free energy of formation is less than or equal to −7 kcal/mol.

173. The method of Paragraph 161, wherein the stem of the at least one stem-loop structure comprises at least eight base pairs.

174. The method of Paragraph 161, wherein the stem of the at least one stem-loop structure comprises at least twenty-five percent GC base pairs.

175. The method of Paragraph 161, wherein the loop of the at least one stem-loop structure comprises at least five nucleotides.

176. The method of Paragraph 161, wherein the nucleotide sequence lacks RNase E recognition sites.

177. The method of Paragraph 161, wherein the at least one stem-loop structure lacks RNase III recognition sites.

178. The method of Paragraph 161, wherein the at least one stem-loop structure lacks a ribosome binding site.

179. The method of Paragraph 161, wherein the at least one stem-loop structure formed at the 3′ end of the antisense nucleic acid comprises at least one rho independent terminator.

180. The method of Paragraph 179, wherein the at least one rho independent terminator comprises rrnBt1 and rrnBt2.

181. The method of Paragraph 161, wherein the sensitized cells are selected from a group consisting of Bacteroides fragilis, Bordetella pertussis, Burkholderia cepacia, Campylobacter jejuni, Chlamydia pneumoniae, Chlamydia trachomatus, Enterobacter cloacae, Escherichia coli, Haemophilus influenzae, Helicobacter pylori, Klebsiella pneumoniae, Neisseria gonorrhoeae, Neisseria meningitidis, Pasteurella haemolytica, Pasteurella multocida, Proteus vulgaris, Pseudomonas aeruginosa, Salmonella bongori, Salmonella cholerasuis, Salmonella enterica, Salmonella paratyphi, Salmonella typhi, Salmonella typhimurium, Moxarella catarrhalis, Shigella boydii, Shigella dysenteriae, Shigella flexneri, Shigella sonnei, Treponema pallidum, Yersinia enterocolitica, Yersinia pestis or any species falling within the genera of any of the above species.

182. The method of Paragraph 161, wherein the sensitized cells are selected from a group consisting of Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species.

183. An isolated or purified nucleic acid comprising a random genomic fragment from a microbial organism flanked on each end by at least one nucleotide sequence which is capable of forming a stem-loop structure.

184. The nucleic acid of Paragraph 183, wherein the nucleic acid further comprises a promoter operably linked thereto.

185. The nucleic acid of Paragraph 183, wherein the promoter is a regulatable promoter.

186. The nucleic acid of Paragraph 185, wherein the promoter is an inducible promoter.

187. The nucleic acid of Paragraph 183, wherein the random genomic fragment is from a gram-negative bacterium.

188. The method of Paragraph 183, wherein the random genomic fragment is from an organism selected from the group consisting of Bacteroides fragilis, Bordetella pertussis, Burkholderia cepacia, Campylobacter jejuni, Chlamydia pneumoniae, Chlamydia trachomatus, Enterobacter cloacae, Escherichia coli, Haemophilus influenzae, Helicobacter pylori, Klebsiella pneumoniae, Neisseria gonorrhoeae, Neisseria meningitidis, Pasteurella haemolytica, Pasteurella multocida, Proteus vulgaris, Pseudomonas aeruginosa, Salmonella bongori, Salmonella cholerasuis, Salmonella enterica, Salmonella paratyphi, Salmonella typhi, Salmonella typhimurium, Moxarella catarrhalis, Shigella boydii, Shigella dysenteriae, Shigella flexneri, Shigella sonnei, Treponema pallidum, Yersinia enterocolitica, Yersinia pestis or any species falling within the genera of any of the above species.

189. The nucleic acid of Paragraph 183, wherein the random genomic fragment is from an organism selected from the group consisting of Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species.

190. A method for screening a candidate antibiotic compound which inhibits the proliferation of a cell said method comprising the steps of:

(a) sensitizing a cell by providing a sublethal level of an antisense nucleic acid complementary to at least a portion of a gene encoding a proliferation-required gene product in said cell, wherein said antisense nucleic acid is flanked on each end by at least one stem-loop structure;

(b) contacting said sensitized cell with a candidate antibiotic compound; and

(c) determining the degree to which said candidate antibiotic compound inhibits proliferation of said sensitized cell relative to a cell which has not been sensitized.

191. The method of Paragraph 190, wherein said at least one stem-loop structure formed at the 5′ end of said antisense nucleic acid comprises a flush, double stranded 5′ end.

192. The method of Paragraph 190, wherein the activity of at least one enzyme involved in RNA degradation has been reduced in said sensitized cell.

193. The method of Paragraph 192, wherein said at least one enzyme involved in RNA degradation is selected from the group consisting of RNase E, RNase II, RNase III, polynucleotide phosphorylase, and poly(A) polymerase.

194. The method of Paragraph 190, wherein said step of sensitizing said cell comprises transcribing said antisense nucleic acid from a promoter.

195. The method of Paragraph 194, wherein said promoter is regulatable.

196. The method of Paragraph 194, wherein the first transcribed nucleotide from said promoter is the first nucleotide of a 5′ stem-loop structure.

197. The method of Paragraph 1, wherein said at least one stem-loop structure comprises SEQ ID NO.: 5.

198. The method of Paragraph 190, wherein said antisense nucleic acid lacks RNase E recognition sites.

199. The method of Paragraph 190, wherein said at least one stem-loop structure lacks RNase III recognition sites.

200. The method of Paragraph 190, wherein said at least one stem-loop structure lacks a ribosome binding site.

201. The method of Paragraph 190, wherein said at least one stem-loop structure formed at the 3′ end of said antisense nucleic acid comprises at least one rho independent terminator.

202. The method of Paragraph 190, wherein said sensitized cell is a gram-negative bacterium.

203. The method of Paragraph 190, wherein said sensitized cell is selected from a group consisting of Bacteroides fragilis, Bordetella pertussis, Burkholderia cepacia, Campylobacter jejuni, Chlamydia pneumoniae, Chlamydia trachomatus, Enterobacter cloacae, Escherichia coli, Haemophilus influenzae, Helicobacter pylori, Klebsiella pneumoniae, Neisseria gonorrhoeae, Neisseria meningitidis, Pasteurella haemolytica, Pasteurella multocida, Proteus vulgaris, Pseudomonas aeruginosa, Salmonella bongori, Salmonella cholerasuis, Salmonella enterica, Salmonella paratyphi, Salmonella typhi, Salmonella typhimurium, Moxarella catarrhalis, Shigella boydii, Shigella dysenteriae, Shigella flexneri, Shigella sonnei, Treponema pallidum, Yersinia enterocolitica, Yersinia pestis or any species falling within the genera of any of the above species.

204. A candidate antibiotic compound identified using the method of Paragraph 190.

205. A method for identifying a gene which is required for proliferation of a cell comprising:

(a) contacting a cell with an antisense nucleic acid flanked on each end by at least one stem-loop structure,

(b) determining whether said antisense nucleic acid inhibits proliferation of said cell; and

(c) identifying the gene in said cell which encodes the mRNA which is complementary to said antisense nucleic acid or a portion thereof.

206. The method of Paragraph 205, wherein said step of determining whether said antisense nucleic acid inhibits the proliferation of said cell comprises comparing the proliferation of said cell transcribing a first level of said antisense nucleic acid to the proliferation of said cell which transcribes a lower level of said antisense nucleic acid or which does not transcribe said antisense nucleic acid.

207. The method of Paragraph 205, wherein said at least one stem-loop structure formed at the 5′ end of said antisense nucleic acid comprises a flush, double stranded 5′ end.

208. The method of Paragraph 205, wherein the activity of at least one enzyme involved in RNA degradation has been reduced in said cell.

209. The method of Paragraph 205, wherein said at least one enzyme involved in RNA degradation is selected from the group consisting of RNase E, RNase II, RNase III, polynucleotide phosphorylase, and poly(A) polymerase.

210. The method of Paragraph 209, wherein said antisense nucleic acid comprises a random genomic fragment from said organism.

211. The method of Paragraph 209, wherein said step of contacting said cell with said antisense nucleic acid comprises transcribing said antisense nucleic acid from a promoter.

212. The method of Paragraph 211, wherein said promoter is regulatable.

213. The method of Paragraph 211, wherein the first transcribed nucleotide from said promoter is the first nucleotide of a 5′ stem-loop structure.

214. The method of Paragraph 205, wherein said at least one stem-loop structure comprises SEQ ID NO.: 5.

215. The method of Paragraph 205, wherein said antisense nucleic acid lacks RNase E recognition sites.

216. The method of Paragraph 205, wherein said at least one stem-loop structure lacks RNase III recognition sites.

217. The method of Paragraph 205, wherein said at least one stem-loop structure lacks a ribosome binding site.

218. The method of Paragraph 205, wherein said at least one stem-loop structure formed at the 3′ end of said antisense nucleic acid comprises at least one rho independent terminator.

219. The method of Paragraph 205, wherein said cell is a gram-negative bacterium.

220. The method of Paragraph 205, wherein said sensitized cells are selected from a group consisting of Bacteroides fragilis, Bordetella pertussis, Burkholderia cepacia, Campylobacter jejuni, Chlamydia pneumoniae, Chlamydia trachomatus, Enterobacter cloacae, Escherichia coli, Haemophilus influenzae, Helicobacter pylori, Klebsiella pneumoniae, Neisseria gonorrhoeae, Neisseria meningitidis, Pasteurella haemolytica, Pasteurella multocida, Proteus vulgaris, Pseudomonas aeruginosa, Salmonella bongori, Salmonella cholerasuis, Salmonella enterica, Salmonella paratyphi, Salmonella typhi, Salmonella typhimurium, Moxarella catarrhalis, Shigella boydii, Shigella dysenteriae, Shigella flexneri, Shigella sonnei, Treponema pallidum, Yersinia enterocolitica, Yersinia pestis or any species falling within the genera of any of the above species.

221. A method for manufacturing an antibiotic comprising the steps of:

(a) contacting sensitized cells which express a sublethal level of an antisense nucleic acid flanked on each end by at least one stem-loop structure with a compound;

(b) identifying a compound which substantially inhibits the proliferation of said sensitized cells relative to cells which have not been sensitized; and

(c) manufacturing the compound so identified.

222. The method of Paragraph 221, wherein said at least one stem-loop structure formed at the 5′ end of said antisense nucleic acid comprises a flush, double stranded 5′ end.

223. The method of Paragraph 221, wherein the activity of at least one enzyme involved in RNA degradation has been reduced in said sensitized cells.

224. The method of Paragraph 221, wherein said antisense nucleic acid comprises a random genomic fragment from said sensitized cells.

225. The method of Paragraph 221, wherein said sensitized cells comprise a gram-negative bacterium.

226. The method of Paragraph 221, wherein said sensitized cells are selected from a group consisting of Bacteroides fragilis, Bordetella pertussis, Burkholderia cepacia, Campylobacter jejuni, Chlamydia pneumoniae, Chlamydia trachomatus, Enterobacter cloacae, Escherichia coli, Haemophilus influenzae, Helicobacter pylori, Klebsiella pneumoniae, Neisseria gonorrhoeae, Neisseria meningitidis, Pasteurella haemolytica, Pasteurella multocida, Proteus vulgaris, Pseudomonas aeruginosa, Salmonella bongori, Salmonella cholerasuis, Salmonella enterica, Salmonella paratyphi, Salmonella typhi, Salmonella typhimurium, Moxarella catarrhalis, Shigella boydii, Shigella dysenteriae, Shigella flexneri, Shigella sonnei, Treponema pallidum, Yersinia enterocolitica, Yersinia pestis or any species falling within the genera of any of the above species.

### DEFINITIONS

The following definitions are provided so as to facilitate the understanding of the invention as set out herein.

By “biological pathway” is meant any discrete cell function or process that is carried out by a gene product or a subset of gene products. Biological pathways include anabolic, catabolic, enzymatic, biochemical and metabolic pathways as well as pathways involved in the production of cellular structures such as cell walls. Biological pathways that are usually required for proliferation of cells or microorganisms include, but are not limited to, cell division, DNA synthesis and replication, RNA synthesis (transcription), protein synthesis (translation), protein processing, protein transport, fatty acid biosynthesis, electron transport chains, cell wall synthesis, cell membrane production, synthesis and maintenance, and the like.

By “inhibit activity of a gene or gene product” is meant having the ability to interfere with the function of a gene or gene product in such a way as to decrease expression of the gene, in such a way as to reduce the level or activity of a product of the gene or in such a way as to inhibit the interaction of the gene or gene product with other biological molecules required for its activity. Agents which inhibit the activity of a gene include agents that inhibit transcription of the gene, agents that inhibit processing of the transcript of the gene, agents that reduce the stability of the transcript of the gene, and agents that inhibit translation of the mRNA transcribed from the gene. In microorganisms, agents which inhibit the activity of a gene can act to decrease expression of the operon in which the gene resides or alter the folding or processing of operon RNA so as to reduce the level or activity of the gene product. The gene product can be a non-translated RNA such as ribosomal RNA, a translated RNA (mRNA) or the protein product resulting from translation of the gene mRNA. Of particular utility to the present invention are antisense RNAs that have activities against the operons or genes to which they specifically hybridize.

By “activity against a gene product” is meant having the ability to inhibit the function or to reduce the level or activity of the gene product in a cell. This includes, but is not limited to, inhibiting the enzymatic activity of the gene product or the ability of the gene product to interact with other biological molecules required for its activity, including inhibiting the gene product's assembly into a multimeric structure.

By “activity against a protein” is meant having the ability to inhibit the function or to reduce the level or activity of the protein in a cell. This includes, but is not limited to, inhibiting the enzymatic activity of the protein or the ability of the protein to interact with other biological molecules required for its activity, including inhibiting the protein's assembly into a multimeric structure.

By “activity against a nucleic acid” is meant having the ability to inhibit the function or to reduce the level or activity of the nucleic acid in a cell. This includes, but is not limited to, inhibiting the ability of the nucleic acid interact with other biological molecules required for its activity, including inhibiting the nucleic acid's assembly into a multimeric structure.

By “activity against a gene” is meant having the ability to inhibit the function or expression of the gene in a cell. This includes, but is not limited to, inhibiting the ability of the gene to interact with other biological molecules required for its activity.

By “activity against an operon” is meant having the ability to inhibit the function or reduce the level of one or more products of the operon in a cell. This includes, but is not limited to, inhibiting the enzymatic activity of one or more products of the operon or the ability of one or more products of the operon to interact with other biological molecules required for its activity.

By “antibiotic” is meant an agent which inhibits the proliferation of a cell or microorganism.

By “E. coli or Escherichia coli” is meant Escherichia coli or any organism previously categorized as a species of Shigella including Shigella boydii, Shigella flexneri, Shigella dysenteriae, Shigella sonnei, Shigella 2A.

The term “expression” is defined as the production of a sense or antisense RNA molecule from a gene, gene fragment, genomic fragment, chromosome, operon or portion thereof. Expression can also be used to refer to the process of peptide or polypeptide synthesis. An expression vector is defined as a vehicle by which a ribonucleic acid (RNA) sequence is transcribed from a nucleic acid sequence carried within the expression vehicle. The expression vector can also contain features that permit translation of a protein product from the transcribed RNA message expressed from the exogenous nucleic acid sequence carried by the expression vector. Accordingly, an expression vector can produce an RNA molecule as its sole product or the expression vector can produce a RNA molecule that is ultimately translated into a protein product.

By “homologous coding nucleic acid” is meant a nucleic acid homologous to a nucleotide sequence encoding a gene product whose activity or level is inhibited by a stabilized antisense nucleic acid identified as described herein or a portion thereof. In some embodiments, the homologous coding nucleic acid may have at least 97%, at least 95%, at least 90%, at least 85%, at least 80%, or at least 70% nucleotide sequence identity to a coding nucleotide sequence identified as described herein and fragments comprising at least 10, 15, 20, 25, 30, 35, 40, 50, 75, 100, 150, 200, 300, 400, or 500 consecutive nucleotides thereof. In other embodiments the homologous coding nucleic acids may have at least 97%, at least 95%, at least 90%, at least 85%, at least 80%, or at least 70% nucleotide sequence identity to a nucleotide sequence complementary to one of the antisense nucleic acid sequences identified as described herein and fragments comprising at least 10, 15, 20, 25, 30, 35, 40, 50, 75, 100, 150, 200, 300, 400, or 500 consecutive nucleotides thereof. Identity may be measured using BLASTN version 2.0 with the default parameters or tBLASTX with the default parameters. (Altschul, S. F. et al. Gapped BLAST and PSI-BLAST: A New Generation of Protein Database Search Programs, Nucleic Acid Res. 25: 3389-3402 (1997), the disclosure of which is incorporated herein by reference in its entirety) Alternatively a “homologuous coding nucleic acid” could be identified by membership of the gene of interest to a functional orthologue cluster. All other members of that orthologue cluster would be considered homologues. Such a library of functional orthologue clusters can be found at http://www.ncbi.nlm.nih.gov/COG. A gene can be classified into a cluster of orthologous groups or COG by using the COGNITOR program available at the above web site, or by direct BLASTP comparison of the gene of interest to the members of the COGs and analysis of these results as described by Tatusov, R. L., Galperin, M. Y., Natale, D. A. and Koonin, E. V. (2000) The COG database: a tool for genome-scale analysis of protein functions and evolution. Nucleic Acids Research v. 28 n. 1, pp33-36.

The term “homologous coding nucleic acid” also includes nucleic acids comprising nucleotide sequences which encode polypeptides having at least 99%, 95%, at least 90%, at least 85%, at least 80%, at least 70%, at least 60%, at least 50%, at least 40% or at least 25% amino acid identity or similarity to a polypeptide encoded by one of the coding nucleotide sequences identified as described herein or to a polypeptpide whose expression is inhibited by a stabilized nucleic acid comprising a nucleotide sequence of one of the antisense nucleic acid sequences identified as described herein or fragments comprising at least 5, 10, 15, 20, 25, 30, 35, 40, 50, 75, 100, or 150 consecutive amino acids thereof as determined using the FASTA version 3.0t78 algorithm with the default parameters. Alternatively, protein identity or similarity may be identified using BLASTP with the default parameters, BLASTX with the default parameters, TBLASTN with the default parameters, or tBLASTX with the default parameters. (Altschul, S. F. et al. Gapped BLAST and PSI-BLAST: A New Generation of Protein Database Search Programs, Nucleic Acid Res. 25: 3389-3402 (1997), the disclosure of which is incorporated herein by reference in its entirety).

The term “homologous coding nucleic acid” also includes coding nucleic acids which hybridize under stringent conditions to a nucleotide sequence complementary to one of the coding nucleic acid sequences identified as described herein and coding nucleic acids comprising nucleotide sequences which hybridize under stringent conditions to a fragment comprising at least 10, 15, 20, 25, 30, 35, 40, 50, 75, 100, 150, 200, 300, 400, or 500 consecutive nucleotides of a nucleic acid sequence complementary to one of the coding nucleic acid sequences identified as described herein. As used herein, “stringent conditions” means hybridization to filter-bound nucleic acid in 6×SSC at about 45° C. followed by one or more washes in 0.1×SSC/0.2% SDS at about 68° C. Other exemplary stringent conditions may refer, e.g., to washing in 6×SSC/0.05% sodium pyrophosphate at 37° C., 48° C., 55° C., and 60° C. as appropriate for the particular probe being used.

The term “homologous coding nucleic acid” also includes coding nucleic acids comprising nucleotide sequences which hybridize under moderate conditions to a nucleotide sequence complementary to one of the coding nucleic acid sequences identified as described herein and coding nucleic acids comprising nucleotide sequences which hybridize under moderate conditions to a fragment comprising at least 10, 15, 20, 25, 30, 35, 40, 50, 75, 100, 150, 200, 300, 400, or 500 consecutive nucleotides of a nucleic acid sequence complementary to one of the coding nucleic acid sequences identified as described herein. As used herein, “moderate conditions” means hybridization to filter-bound DNA in 6×sodium chloride/sodium citrate (SSC) at about 45° C. followed by one or more washes in 0.2×SSC/0.1% SDS at about 42-65° C.

The term “homologous coding nucleic acids” also includes nucleic acids comprising nucleotide sequences which encode a gene product whose activity may be complemented by a gene encoding a gene product whose activity is inhibited by a nucleic acid comprising a stabilized antisense nucleotide sequence identified as described herein. In some embodiments, the homologous coding nucleic acids may encode a gene product whose activity is complemented by the gene product encoded by a nucleic acid comprising a coding nucleotide sequence identified as described herein.

The term “homologous antisense nucleic acid” includes nucleic acids comprising a nucleotide sequence having at least 97%, at least 95%, at least 90%, at least 85%, at least 80%, or at least 70% nucleotide sequence identity to an antisense nucleotide sequence identified as described herein and fragments comprising at least 10, 15, 20, 25, 30, 35, 40, 50, 75, 100, 150, 200, 300, 400, or 500 consecutive nucleotides thereof. Homologous antisense nucleic acids may also comprise nucleotide sequences which have at least 97%, at least 95%, at least 90%, at least 85%, at least 80%, or at least 70% nucleotide sequence identity to a nucleotide sequence complementary to one of the coding nucleic acid sequences identified as described herein and fragments comprising at least 10, 15, 20, 25, 30, 35, 40, 50, 75, 100, 150, 200, 300, 400, or 500 consecutive nucleotides thereof. Nucleic acid identity may be determined as described above.

The term “homologous antisense nucleic acid” also includes antisense nucleic acids comprising nucleotide sequences which hybridize under stringent conditions to a nucleotide sequence complementary to one of antisense nucleic acid sequences identified as described herein and antisense nucleic acids comprising nucleotide sequences which hybridize under stringent conditions to a fragment comprising at least 10, 15, 20, 25, 30, 35, 40, 50, 75, 100, 150, 200, 300, 400, or 500 consecutive nucleotides of the sequence complementary to one of the antisense nucleic acid sequences identified as described herein. Homologous antisense nucleic acids also include antisense nucleic acids comprising nucleotide sequences which hybridize under stringent conditions to a coding nucleotide sequence identified as described herein and antisense nucleic acids comprising nucleotide sequences which hybridize under stringent conditions to a fragment comprising at least 10, 15, 20, 25, 30, 35, 40, 50, 75, 100, 150, 200, 300, 400, or 500 consecutive nucleotides of one of the coding nucleic acid sequences identified as described herein.

The term “homologous antisense nucleic acid” also includes antisense nucleic acids comprising nucleotide sequences which hybridize under moderate conditions to a nucleotide sequence complementary to one of the antisense nucleic acid sequences identified as described herein and antisense nucleic acids comprising nucleotide seuqences which hybridize under moderate conditions to a fragment comprising at least 10, 15, 20, 25, 30, 35, 40, 50, 75, 100, 150, 200, 300, 400, or 500 consecutive nucleotides of the sequence complementary to one of the antisense nucleic acid sequences identified as described herein. Homologous antisense nucleic acids also include antisense nucleic acids comprising nucleotide seuqences which hybridize under moderate conditions to a coding nucleotide sequence identified as described herein and antisense nucleic acids which comprising nucleotide sequences hybridize under moderate conditions to a fragment comprising at least 10, 15, 20, 25, 30, 35, 40, 50, 75, 100, 150, 200, 300, 400, or 500 consecutive nucleotides of one of the coding nucleic acid sequences identified as described herein.

By “homologous polypeptide” is meant a polypeptide homologous to a polypeptide whose activity or level is inhibited by a nucleic acid comprising a stabilized antisense nucleotide sequence identified as described herein or by a homologous antisense nucleic acid. The term “homologous polypeptide” includes polypeptides having at least 99%, 95%, at least 90%, at least 85%, at least 80%, at least 70%, at least 60%, at least 50%, at least 40% or at least 25% amino acid identity or similarity to a polypeptide whose activity or level is inhibited by a stabilized antisense nucleic acid identified as described herein or by a homologous antisense nucleic acid, or polypeptides having at least 99%, 95%, at least 90%, at least 85%, at least 80%, at least 70%, at least 60%, at least 50%, at least 40% or at least 25% amino acid identity or similarity to a polypeptide to a fragment comprising at least 5, 10, 15, 20, 25, 30, 35, 40, 50, 75, 100, or 150 consecutive amino acids of a polypeptide whose activity or level is inhibited by a stabilized antisense nucleic acid identified as described herein or by a homologous antisense nucleic acid. Identity or similarity may be determined using the FASTA version 3.0t78 algorithm with the default parameters. Alternatively, protein identity or similarity may be identified using BLASTP with the default parameters, BLASTX with the default parameters, or TBLASTN with the default parameters. (Altschul, S. F. et al. Gapped BLAST and PSI-BLAST: A New Generation of Protein Database Search Programs, Nucleic Acid Res. 25: 3389-3402 (1997), the disclosure of which is incorporated herein by reference in its entirety).

The term “homologous polypeptide” also includes polypeptides having at least 99%, 95%, at least 90%, at least 85%, at least 80%, at least 70%, at least 60%, at least 50%, at least 40% or at least 25% amino acid identity or similarity to a polypeptide encoded by one of the coding nucleic acid sequences identified as described herein and polypeptides having at least 99%, 95%, at least 90%, at least 85%, at least 80%, at least 70%, at least 60%, at least 50%, at least 40% or at least 25% amino acid identity or similarity to a fragment comprising at least 5, 10, 15, 20, 25, 30, 35, 40, 50, 75, 100, or 150 consecutive amino acids of a polypeptide encoded by one of the coding nucleic acid sequences identified as described herein.

The invention also includes polynucleotides, preferably DNA molecules, that hybridize to one of the antisense nucleic acid sequences identified as described herein, coding nucleic acid sequences identified as described herein or the complements of any of the preceding nucleic acids. Such hybridization may be under stringent or moderate conditions as defined above or under other conditions which permit specific hybridization. The nucleic acid molecules of the invention that hybridize to these DNA sequences include oligodeoxynucleotides (“oligos”) which hybridize to the target gene under highly stringent or stringent conditions. In general, for oligos between 14 and 70 nucleotides in length the melting temperature (Tm) is calculated using the formula:

Tm(° C.)=81.5+16.6(log[monovalent cations (molar)]+0.41 (% G+C)−(500/N)

where N is the length of the probe. If the hybridization is carried out in a solution containing formamide, the melting temperature may be calculated using the equation:

Tm(° C.)=81.5+16.6(log[monovalent cations (molar)]+0.41(% G+C)−(0.61) (% formamide)−(500/N)

where N is the length of the probe. In general, hybridization is carried out at about 20-25 degrees below Tm (for DNA-DNA hybrids) or about 10-15 degrees below Tm (for RNA-DNA hybrids).

Other hybridization conditions are apparent to those of skill in the art (see, for example, Ausubel, F. M. et al., eds., 1989, Current Protocols in Molecular Biology, Vol. I, Green Publishing Associates, Inc. and John Wiley & Sons, Inc., New York, at pp. 6.3.1-6.3.6 and 2.10.3, the disclosure of which is incorporated herein by reference in its entirety).

The term, Salmonella, is the generic name for a large group of gram-negative enteric bacteria that are closely related to Escherichia coli. The diseases caused by Salmonella are often due to contamination of foodstuffs or the water supply and affect millions of people each year. Traditional methods of Salmonella taxonomy were based on assigning a separate species name to each serologically distinguishable strain (Kauffmann, F 1966 The bacteriology of the Enterobacteriaceae. Munksgaard, Copenhagen). Serology of Salmonella is based on surface antigens (O [somatic] and H [flagellar]). Over 2,400 serotypes or serovars of Salmonella are known (Popoff, et al. 2000 Res. Microbiol. 151:63-65). Therefore, each serotype was considered to be a separate species and often given names, accordingly (e.g. S. paratyphi, S. typhimurium, S. typhi, S. enteriditis, etc.).

However, by the 1970s and 1980s it was recognized that this system was not only cumbersome, but also inaccurate. Then, many Salmonella species were lumped into a single species (all serotypes and subgenera I, II, and IV and all serotypes of Arizona) with a second subspecies, S. bongorii also recognized (Crosa, et al., 1973, J. Bacteriol. 115:307-315). Though species designations are based on the highly variable surface antigens, the Salmonella are very similar otherwise with a major exception being pathogenicity determinants.

There has been some debate on the correct name for the Salmonella species. Currently (Brenner, et al. 2000 J. Clin. Microbiol. 38:2465-2467), the accepted name is Salmonella enterica. S. enterica is divided into six subspecies (I, S. enterica subsp. enterica; II, S. enterica, subsp. salamae; IIIa, S. enterica subsp. arizonàe; IIIb, S. enterica subsp. diarizonae; IV, S. enterica subsp. houtenae; and VI, S. enterica subsp. indica). Within subspecies I, serotypes are used to distinguish each of the serotypes or serovars (e.g. S. enterica serotype Enteriditis, S. enterica serotype Typhimurium, S. enterica serotype Typhi, and S. enterica serotype Choleraesuis, etc.). Current convention is to spell this out on first usage (Salmonella enterica ser. Typhimurium) and then use an abbreviated form (Salmonella Typhimurium or S. Typhimurium). Note, the genus and species names (Salmonella enterica) are italicized but not the serotype/serovar name (Typhimurium). Because the taxonomic committees have yet to officially approve of the actual species name, this latter system is what is employed by the CDC (Brenner, et al. 2000 J. Clin. Microbiol. 38:2465-2467). Due to the concerns of both taxonomic priority and medical importance, some of these serotypes might ultimately receive full species designations (S. typhi would be the most notable).

Therefore, as used herein “Salmonella enterica or S. enterica” includes serovars Typhi, Typhimurium, Paratyphi, Choleraesuis, etc. However, appeals of the “official” name are in process and the taxonomic designations may change (S. choleraesuis is the species name that could replace S. enterica based solely on priority).

By “identifying a compound” is meant to screen one or more compounds in a collection of compounds such as a combinatorial chemical library or other library of chemical compounds or to characterize a single compound by testing the compound in a given assay and determining whether it exhibits the desired activity.

By “inducer” is meant an agent or solution which, when placed in contact with a cell or microorganism, increases transcription, or inhibitor and/or promoter clearance/fidelity, from a desired promoter.

As used herein, “nucleic acid” means DNA, RNA, or modified nucleic acids. Thus, the terminology “the nucleic acid of SEQ ID NO: X” or “the nucleic acid comprising the nucleotide sequence” includes both the DNA sequence of SEQ ID NO: X and an RNA sequence in which the thymidines in the DNA sequence have been substituted with uridines in the RNA sequence and in which the deoxyribose backbone of the DNA sequence has been substituted with a ribose backbone in the RNA sequence. Modified nucleic acids are nucleic acids having nucleotides or structures which do not occur in nature, such as nucleic acids in which the internucleotide phosphate residues with methylphosphonates, phosphorothioates, phosphoramidates, and phosphate esters. Nonphosphate internucleotide analogs such as siloxane bridges, carbonate brides, thioester bridges, as well as many others known in the art may also be used in modified nucleic acids. Modified nucleic acids may also comprise, α-anomeric nucleotide units and modified nucleotides such as 1,2-dideoxy-d-ribofuranose, 1,2-dideoxy-1-phenylribofuranose, and N⁴, N⁴-ethano-5-methyl-cytosine are contemplated for use in the present invention. Modified nucleic acids may also be peptide nucleic acids in which the entire deoxyribose-phosphate backbone has been exchanged with a chemically completely different, but structurally homologous, polyamide (peptide) backbone containing 2-aminoethyl glycine units.

As used herein, “polynucleotide” has the same meaning as nucleic acid.

As used herein, “proliferation-inhibiting” encompasses instances where the absence or substantial reduction of a gene transcript and/or gene product completely eliminates cell growth as well as instances where the absence of a gene transcript and/or gene product merely reduces cell growth. A proliferation-inhibiting antisense nucleic acid is one that can cause a reduction of a gene transcript and/or gene product that is sufficient to reduce or eliminate the growth or viability of the cell or microorganism.

As used herein, “proliferation-required” or “required for proliferation” encompasses instances where the absence or substantial reduction of a gene transcript and/or gene product completely eliminates cell growth as well as instances where the absence of a gene transcript and/or gene product merely reduces cell growth. A proliferation-required gene or gene family is one where, in the absence or substantial reduction of a gene transcript and/or gene product, growth or viability of the cell or microorganism is reduced or eliminated.

As used herein, “stabilize” means to increase resistance to degradation or decomposition.

As used herein, “sub-lethal” means a concentration of an agent below the concentration required to inhibit all cell growth.

### BRIEF DESCRIPTION OF THE DRAWINGS

The foregoing aspects and many of the attendant advantages of this invention will become more readily appreciated as the same becomes better understood by reference to the following detailed description, when taken in conjunction with the accompanying drawings, wherein:

FIG. 1 is an illustration which shows the structure of the flush 5′ stem-loop region (SEQ ID NO.: 5) and the 3′ stem loop of an RNA transcript produced by pEPEC3.

FIG. 2 is an autoradiogram of a Northern blot which shows the stability of RNA transcripts having stem-loop structures at either one or both ends.

FIG. 3 is an autoradiogram of a Northern blot which compares the stability of RNA transcripts having stem-loop structures at each end in wildtype cells and cells that have reduced RNase E function.

FIG. 4a is a graph which shows the fold increase in sensitivity to stabilized rplW antisense transcript for E. coli strains having mutations affecting one or more enzymes involved in RNA degradation. The increase in sensitivity was determined by comparing the effect of the rplW antisense transcript on the proliferation ability of each of the mutant strains with its effect on the wildtype.

FIG. 4b is a graph which shows the fold increase in sensitivity to stabilized rplL, rplJ antisense transcript for E. coli strains having mutations affecting one or more enzymes involved in RNA degradation. The increase in sensitivity was determined by comparing the effect of the rplL, rplJ antisense transcript on the proliferation ability of each of the mutant strains with its effect on the wildtype.

FIG. 4c is a graph which shows the fold increase in sensitivity to stabilized glnS antisense transcript for E. coli strains having mutations affecting one or more enzymes involved in RNA degradation. The increase in sensitivity was determined by comparing the effect of the tRNA synthetase antisense transcript on the proliferation ability of each of the mutant strains with its effect on the wildtype.

FIG. 5 is an IPTG dose response curve in E. coli transformed with an IPTG-inducible plasmid containing either an antisense clone to the E. coli gene encoding the ribosomal protein rplW (AS-rplW) which is required for protein synthesis and essential cell proliferation, or an antisense clone to the elaD (AS-elaD) gene which is not known to be involved in protein synthesis and which is also essential for proliferation.

FIG. 6A is a tetracycline dose response curve in E. coli transformed with an IPTG-inducible plasmid containing antisense to rplW(AS-rplW) in the presence of 0, 20 or 50 μM IPTG.

FIG. 6B is a tetracycline dose response curve in E. coli transformed with an IPTG-inducible plasmid containing antisense to elaD (AS-elaD) in the presence of 0, 20 or 50 μM IPTG.

FIG. 7 is a graph showing the fold increase in tetracycline sensitivity of E. coli transfected with antisense clones to essential ribosomal proteins L23 (AS-rplW) and L7/L12 and L10 (AS-rplLrplJ). Antisense clones to genes known not to be involved in protein synthesis (atpB/E(AS-atpB/E), visC (AS-visC), elaD (AS-elaD), yohH (AS-yohH)) are much less sensitive to tetracycline.

### DETAILED DESCRIPTION OF THE INVENTION

Current methods which employ antisense nucleic acids in the discovery of novel proliferation-required genes and novel antibiotics effecting the expression of those genes have been largely successful. Such methods have been described in U.S. Pat. No. 6,228,579 and International Publication Nos., WO 00/44906, WO 01/34810, WO 01/48209 and WO 01/70955, the disclosures of which are incorporated herein in their entireties. However, the full potential of such techniques has not been reached due to, at least in part, the inherent instability of certain antisense nucleic acid constructs, particularly in gram-negative bacteria. The present invention describes methods for increasing the stability of nucleic acids that are used in applications such as drug discovery and the identification of proliferation-required genes in organisms.

One aspect of the present invention contemplates utilizing degradation-resistant antisense nucleic acids in the identification of proliferation-required genes. In one embodiment, a library of genomic fragments from an organism are subcloned or otherwise inserted immediately downstream of a regulatable promoter, such as an inducible promoter, in an expression vector which has been engineered to produce degradation-resistant RNA transcripts. In one embodiment, the genomic fragments are random fragments. The vector may be engineered to have at least one nucleic acid sequence which forms a stem-loop structure when transcribed (referred to herein as “stem-loop-encoding sequences”) flanking each end of the nucleotide sequence encoding the antisense RNA. In some embodiments the transcrips may have a plurality of stem-loop structures at one or both ends of the antisense RNA. For example, these transcripts may have at leaset two, at three, at least five, or more than five stem-loop structures at one or both ends of the antisense RNA. For example, in some embodiments, the stem-loop-encoding sequences flank each end of a multiple cloning site (MCS) in the vector into which the nucleotide sequence encoding the antisense RNA is inserted. In some embodiments, the stem-loop-encoding nucleotide sequence that is located downstream of the MCS may be produced by including a nucleic acid segment which encodes a rho-independent terminator in the vector. Rho-independent terminators are well known to those of skill in the art and any rho-independent terminator may be used with the present invention. Thus, the rho-independent terminator serves the dual function of terminating transcription and increasing the stability of the RNA transcript. The second stem-loop-encoding sequence is provided by a nucleic acid segment that is located upstream of the MCS. This segment can include the transcription initiation site. Accordingly, an RNA transcript produced from this vector, will comprise the inserted polynucleotide region flanked on each end by nucleic acid segments capable of forming stem-loop structures. In a preferred embodiment of the present invention, the first nucleotide of the transcript comprises the first nucleotide of the 5′ stem-loop structure and this nucleotide is paired with a complementary nucleotide at the base of the stem so as to create a transcript having a flush double stranded 5′ end. In other emobdiments of the present invention, the last nucleotide of the transcript is paired with a complementary nucleotide at the base of the stem so as to create a transcript having a flush double stranded 3′ end. In still further embodiments, the transcript has a flush double stranded 5′ end and a flush double stranded 3′ end.

It is generally preferred that the structural integrity of the stem-loop regions of each transcript be maintained within the cell. Several methods can be used, alone or in combination, to assist in the preservation of the integrity of these stem-loops structures. For example, increasing the G/C content of the stem encoding region decreases the likelihood of disassociation of the paired bases in the stems of these structures. Stem-loop structures having stems comprising at least 15%, at least 25%, at least 35%, at least 45%, at least 55%, at least 65%, at least 75%, or at least 85% G/C content are contemplated. Increasing the number of nucleotides in the stem region has a similar stabilizing effect. For example, stem-loop structures may have stems which comprise at least 6, at least 7, at least 8, at least 9, at least 11, at least 15, or at least 30 base pairs. Additionally, the size of the loop affects the integrity of the stem-loop structure. For example, stem-loop structures may have loops of at least 3, at least 4, at least 5, at least 6, at least 8, at least 10, at least 15, at least 20 nucleotides, or more than 20 nucleotides. In certain embodiments of the present invention, stem-loop structures having a loop region of at least five nucleotides, a stem comprised of at least eight base pairs and having at least twenty-five percent G/C content are contemplated.

The methods used to preserve the integrity of stem-loop structures can also decrease their free energy of formation. For example, the free energy of formation of stem-loop structures may be less than or equal to −3 kcal/mol, less than or equal to −5 kcal/mol, less than or equal to −7 kcal/mol, less than or equal to −9 kcal/mol, less than or equal to −15 kcal/mol, less than or equal to −25 kcal/mol, less than or equal to −40 kcal/mol, or more than −40 kcal/mol. In particular, stem-loop structures having a free energy of formation of less than or equal to −7 kcal/mol are contemplated.

In addition to the methods described above, other sequence-based strategies can be employed to preserve stem-loop integrity. For example, the structural integrity of stem-loop structures within the cell maybe enhanced ensuring that the transcripts comprising the antisense nucleic acid flanked on each end by at least one stem-loop-encoding sequence are free of recognition sites for enzymes involved in RNA degradation. For example, the transcripts may be free of recognition sites for RNase E, RNase II, RNase III, polynucleotide phosphorylase, and poly (A) polymerase. Additionally or alternatively, structural integrity can be enhanced by excluding ribosomal binding sites from stem-loop-encoding regions. In a preferred embodiment of the current invention, the stem-loop structures in the transcripts are free of ribosomal binding sites as well as recognition sites for enzymes involved in RNA degradation, such as recognition sites for RNase E, RNase II, RNase III, polynucleotide phosphorylase, and poly (A) polymerase.

It is generally preferred that expression of the stabilized RNA transcripts is directed by a regulatable promoter sequence such that expression level can be adjusted by addition of variable concentrations of an inducer molecule or of an inhibitor molecule to the medium. Temperature activated promoters, such as promoters regulated by temperature sensitive repressors, such as the lambda C₁₈₅₇ repressor, are also envisioned. Although the insert nucleic acids may be derived from the chromosome of the cell or microorganism into which the expression vector is to be introduced, because the insert is not in its natural chromosomal location, the insert nucleic acid is an exogenous nucleic acid for the purposes of the discussion herein.

Once generated, the vectors comprising the genomic fragments flanked on each end by at least one stem-loop-encoding sequence operably linked to a regulatable promoter are introduced into a population of cells (such as the organism from which the exogenous nucleic acid sequences (i.e. the genomic fragments) were obtained) to identify genes that are required for proliferation. In some embodiemtns the cells are bacterial cells. The bacterial cells may be gram-positive or gram-negative. The extent of proliferation of cells grown under conditions in which the antisense nucleic acid is expressed at a first level is compared to the extent of proliferation of cells in which the antisense nucleic acid is expressed at a second level which is lower than the first level or to the extent of proliferation of cells in which the antisense nucleic acid is not expressed. If the cells expressing the first level of antisense nucleic acid proliferate significantly less than the cells which express the antisense nucleic acid at a lower level or which do not express the antisense nucleic acid, the antisense nucleic acid is complementary to at least a portion of a gene which is required for proliferation.

In some embodiments, the host strain into which the vectors are introduced can be chosen so as to further aid in the stabilization of the RNA molecules transcribed from the expression vector. For example, in some embodiments, strains which have a reduced ability to degrade RNA are used as expression hosts. Thus, in some embodiments, the vectors are introduced into strains having one or more mutations in a gene or genes that encode one or more enzymes involved in the degradation of RNA. In some embodiments, the reduction in RNA degradation ability of these strains can be due to the reduction in the activity of one or more enzymes that directly cleave RNA molecules. Such enzymes include but are not limited to RNase E, RNase II, RNase III, and polynucleotide phosphorylase. In other embodiments, the activity of one or more enzymes that are peripherally involved in RNA degradation such as poly (A) polymerase and RNA helicase may be reduced in the host strain. Additionally or alternatively, the vectors may be introduced into host strains in which the activity of one or more enzymes, such as enolase, which are involved in RNA degradation complexes has been reduced. In a preferred embodiment, the vectors are introduced into host strains in which the activities of multiple enzymes involved in RNA degradation have been reduced.

Transcription of the genomic DNA inserts in the test population of cells containing the expression library of vectors containing the genomic DNA inserts is then activated. In a preferred embodiment, the genomic DNA inserts flanked on each end by at least one stem-loop-encoding sequence are transcribed from a vector capable of producing stabilized transcripts in a host strain having a reduced ability to degrade RNA.

The test population of cells is then assayed to determine the effect of expressing the genomic DNA inserts flanked on each end by at least one stem-loop-encoding sequence on the test population of cells. Those expression vectors that negatively impact the growth of the cells upon induction of expression of the random genomic sequences contained therein are identified, isolated, and purified for further study.

A variety of assays are contemplated to identify nucleic acid sequences that negatively impact growth upon expression. In one embodiment of the present invention, growth in cultures transcribing the genomic DNA inserts flanked on each end by at least one stem-loop-encoding sequence and growth in cultures not transcribing these sequences or transcribing these sequences at a lower level is compared. Growth measurements are assayed by examining the extent of growth by measuring optical densities. Alternatively, enzymatic assays can be used to measure bacterial growth rates to identify genomic inserts of interest. Colony size, colony morphology, and cell morphology are additional factors used to evaluate growth of the host cells. Those cultures that fail to grow or grow at a reduced rate under expression conditions are identified as containing an expression vector encoding a nucleic acid fragment that negatively affects a proliferation-required gene.

Once genomic inserts of interest are identified, they are analyzed. The first step of the analysis is to acquire the nucleotide sequence of the nucleic acid fragment of interest. To achieve this end, the insert in those expression vectors identified as containing a nucleotide sequence of interest is sequenced, using standard techniques well known in the art. The next step of the process is to determine the source of the nucleotide sequence. As used herein “source” means the genomic region containing the cloned fragment.

Determination of the gene(s) corresponding to the nucleotide sequence is achieved by comparing the obtained nucleotide sequence data with databases containing known protein and nucleotide sequences from various microorganisms. Thus, initial gene identification is made on the basis of significant sequence similarity or identity to characterized or predicted genes from the organism under investigation, proteins encoded by those genes, and/or homologues in other species.

The number of nucleotide and protein sequences available in database systems has been growing exponentially for years. For example, the complete nucleotide sequences of Caenorhabditis elegans and several bacterial genomes, including E. coli, Aeropyrum pernix, Aquifex aeolicus, Archaeoglobus fulgidus, Bacillus subtilis, Borrelia burgdorferi, Chlamydia pneumoniae, Chlamydia trachomatis, Clostridium tetani, Corynebacterium diptheria, Deinococcus radiodurans, Haemophilus influenzae, Helicobacter pylori 26695, Helicobacter pylori J99, Methanobacterium thermoautotrophicum, Methanococcus jannaschii, Mycobacterium tuberculosis, Mycoplasma genitalium, Mycoplasma pneumoniae, Pseudomonas aeruginosa, Pyrococcus abyssi, Pyrococcus horikoshii, Rickettsia prowazekii, Synechocystis PCC6803, Thermotoga maritima, Treponema pallidum, Bordetella pertussis, Campylobacter jejuni, Clostridium acetobutylicum, Mycobacterium tuberculosis CSU#93, Neisseria gonorrhoeae, Neisseria meningitidis, Pseudomonas aeruginosa, Pyrobaculum aerophilum, Pyrococcus furiosus, Rhodobacter capsulatus, Salmonella typhimurium, Streptococcus mutans, Streptococcus pyogenes, Ureaplasma urealyticum and Vibrio cholera are available. This nucleotide sequence information is stored in a number of databanks, such as GenBank, the National Center for Biotechnology Information (NCBI), the Genome Sequencing Center (http://genome.wustl.edu/gsc/salmonella.shtml), and the Sanger Centre (http://www.sanger.ac.uk/projects/S_{—}typhi) which are publicly available for searching. A variety of computer programs are available to assist in the analysis of the sequences stored within these databases. FASTA, (W. R. Pearson (1990) “Rapid and Sensitive Sequence Comparison with FASTP and FASTA” Methods in Enzymology 183:63-98), Sequence Retrieval System (SRS), (Etzold & Argos, SRS an indexing and retrieval tool for flat file data libraries. Comput. Appl. Biosci. 9:49-57, 1993) are two examples of computer programs that can be used to analyze sequences of interest. In one embodiment of the present invention, the BLAST family of computer programs, which includes BLASTN version 2.0 with the default parameters, or BLASTX version 2.0 with the default parameters, is used to analyze nucleotide sequences.

BLAST, an acronym for “Basic Local Alignment Search Tool,” is a family of programs for database similarity searching. The BLAST family of programs includes: BLASTN, a nucleotide sequence database searching program, BLASTX, a protein database searching program where the input is a nucleic acid sequence; and BLASTP, a protein database searching program. BLAST programs embody a fast algorithm for sequence matching, rigorous statistical methods for judging the significance of matches, and various options for tailoring the program for special situations. Assistance in using the program can be obtained by e-mail at blast@ncbi.nlm.nih.gov. tBLASTX can be used to translate a nucleotide sequence in all three potential reading frames into an amino acid sequence.

Bacterial genes are often transcribed in polycistronic groups. These groups comprise operons, which are a collection of genes and intergenic sequences under common regulation. The genes of an operon are transcribed on the same mRNA and are often related functionally. Given the nature of the screening protocol, it is possible that the identified genomic insert corresponds to a gene or portion thereof with or without adjacent noncoding sequences, an intragenic sequence (i.e. a sequence within a gene), an intergenic sequence (i.e. a sequence between genes), a nucleotide sequence spanning at least a portion of two or more genes, a 5′ noncoding region or a 3′ noncoding region located upstream or downstream from the actual nucleotide sequence that is required for bacterial proliferation. Accordingly, it is often desirable to determine which gene(s) that is encoded within the operon is individually required for proliferation.

In one embodiment of the present invention, an operon is identified and then dissected to determine which gene or genes are required for proliferation. Operons can be identified by a variety of means known to those in the art. For example, the RegulonDB DataBase described by Huerta et al. (Nucl. Acids Res. 26:55-59, 1998), which may also be found on the website http://www.cifn.unam.mx/Computational_Biology/regulondb/, the disclosures of which are incorporated herein by reference in their entireties, provides information about operons in Escherichia coli. The Subtilist database (http://bioweb.pasteur.fr/GenoList/SubtiList), (Moszer, I., Glaser, P. and Danchin, A. (1995) Microbiology 141: 261-268 and Moszer, 1 (1998) FEBS Letters 430: 28-36, the disclosures of which are incorporated herein in their entireties), may also be used to predict operons. This database lists genes from the fully sequenced, Gram-positive bacteria, Bacillus subtilis, together with predicted promoters and terminator sites. The Pseudomonas aeruginosa web site (http://www.pseudomonas.com) can be used to help predict operon organization in this bacterium. The databases available from the Genome Sequencing Center (http://genome.wustl.edu/gsc/salmonella.shtml), and the Sanger Centre (http://www.sanger.ac.uk/projects/S_{—}typhi) may be used to predict operons in Salmonella typhimurium. The TIGR microbial database has an incomplete version of the E. faecalis genome http://www.tigr.org/cgi-bin/BlastSearch/blast.cgi?organism=e_{—}faecalis. One can take a nucleotide sequence and BLAST it for homologs.

A number of techniques that are well known in the art can be used to dissect the operon. Analysis of RNA transcripts by Northern blot or primer extension techniques are commonly used to analyze operon transcripts. In one aspect of this embodiment, gene disruption by homologous recombination is used to individually inactivate the genes of an operon that is thought to contain a gene required for proliferation.

Several gene disruption techniques have been described for the replacement of a functional gene with a mutated, non-functional (null) allele. These techniques generally involve the use of homologous recombination. One technique such technique uses crossover PCR to create a null allele with an in-frame deletion of the coding region of a target gene. The null allele is constructed in such a way that nucleotide sequences adjacent to the wild type gene are retained. These homologous nucleotide sequences surrounding the deletion null allele provide targets for homologous recombination so that the wild type gene on the bacterial chromosome can be replaced by the constructed null allele. This method can be used with a number of microbes, including Staphylococcus, Salmonella and Klebsiella species. Similar gene disruption methods that employ the counter selectable marker sacB (Schweizer, H. P., Klassen, T. and Hoang, T. (1996) Mol. Biol. of Pseudomonas. ASM press, 229-237, the disclosure of which is incorporated herein by reference in its entirety) are available for Pseudomonas, Salmonella and Klebsiella species. E. faecalis genes can be disrupted by recombining in a non-replicating plasmid that contains an internal fragment to that gene (Leboeuf, C., L. Leblanc, Y. Auffray and A. Hartke. 2000. J. Bacteriol. 182:5799-5806, the disclosure of which is incorporated herein by reference in its entirety).

The crossover PCR amplification product is subcloned into a suitable vector having a selectable marker, such as a drug resistance marker. In some embodiments the vector may have an origin of replication which is functional in E. coli or another organism distinct from the organism in which homologous recombination is to occur, allowing the plasmid to be grown in E. coli or the organism other than that in which homologous recombination is to occur, but may lack an origin of replication that is functional in the organism in which homologous recombination is to occur such that selection of the selectable marker requires integration of the vector into the homologous region of the chromosome of the organism in which homologous replication is to occur. Usually a single crossover event is responsible for this integration event such that the chromosome of the crossover host now contains a tandem duplication of the target gene consisting of one wild type allele and one deletion null allele separated by vector sequence. Subsequent resolution of the duplication results in both removal of the vector sequence and either restoration of the wild type gene or replacement by the in-frame deletion. The latter outcome will not occur if the gene should prove essential. A more detailed description of this method is provided in Example 10 below.

In another aspect, the present invention describes methods for identification of nucleotide sequences homologous to genes identified as described herein. The present invention also describes methods for identifying polypeptides homologous to polypeptides encoded by the genes identified as described herein. For example, the genes identified as described herein may be used to identify homologous coding nucleic acids, homologous antisense nucleic acids, or homologous polypeptides in microorganisms such as Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosai, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species.

The homologous coding nucleic acids, homologous antisense nucleic acids, or homologous polypeptides, may then be used in each of the methods described herein, including methods to identify compounds which inhibit the proliferation of the organism containing the homologous coding nucleic acid, homologous antisense nucleic acid or homologous polypeptide, methods of inhibiting the growth of the organism containing the homologous coding nucleic acid, homologus antisense nucleic acid or homologous polypeptide, methods of identifying compounds which influence the activity or level of a gene product required for proliferation of the organism containing the homologous coding nucleic acid, homologous antisense nucleic acid or homologous polypeptide, methods for identifying compounds or nucleic acids having the ability to reduce the level or activity of a gene product required for proliferation of the organism containing the homologous coding nucleic acid, homologous antisense nucleic acid or homologous polypeptide, methods of inhibiting the activity or expression of a gene in an operon required for proliferation of the organism containing the homologous coding nucleic acid, homologous antisense nucleic acid or homologous polypeptide, methods for identifying a gene required proliferation of the organism containing the homologous coding nucleic acid, homologous antisense nucleic acid or homologous polypeptide, methods for identifying the biological pathway in which a gene or gene product required for proliferation of the organism containing the homologous coding nucleic acid, homologous antisense nucleic acid or homologous polypeptide lies, methods for identifying compounds having activity against biological pathway required for proliferation of the organism containing the homologous coding nucleic acid, homologous antisense nucleic acid or homologous polypeptide, methods for determining the biological pathway on which a test compound acts, and methods of inhibiting the proliferation of the organism containing the homologous coding nucleic acid, homologous antisense nucleic acid or homologous polypeptide in a subject. In some embodiments of the present invention, the methods are performed in gram-negative organisms.

The nucleic acid sequences identified as described herein can be used to identify homologous coding nucleic acids or homologous polypeptides required for proliferation from these and other organisms using methods such as nucleic acid hybridization and computer database analysis.

In one embodiment of the present invention, the nucleic acid sequences identified as described herein are used to screen genomic libraries generated from bacterial species of interest. For example, the genomic library may be from gram-positive bacteria, gram-negative bacteria or other organisms including Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species. Standard molecular biology techniques are used to generate genomic libraries from various cells or microorganisms. In one aspect, the libraries are generated and bound to nitrocellulose paper. Nucleic acid sequences identified as described herein can then be used as probes to screen the libraries for homologous nucleotide sequences.

For example, the libraries may be screened to identify homologous coding nucleic acids or homologous antisense nucleic acids comprising nucleotide sequences which hybridize under stringent conditions to an antisense nucleic acid identified as described herein, nucleic acids comprising nucleotide sequences which hybridize under stringent conditions to a fragment comprising at least 10, 15, 20, 25, 30, 35, 40, 50, 75, 100, 150, 200, 300, 400, or 500 consecutive nucleotides of one of the antisense nucleic acids identified as described herein, nucleic acids comprising nucleotide sequences which hybridize under stringent conditions to a nucleic acid complementary to one of the antisense nucleic acids identified as described herein, nucleic acids comprising nucleotide sequences which hybridize under stringent conditions to a fragment comprising at least 10, 15, 20, 25, 30, 35, 40, 50, 75, 100, 150, 200, 300, 400, or 500 consecutive nucleotides of the sequence complementary to one of the antisense nucleic acids identified as described herein, nucleic acids comprising nucleotide sequences which hybridize under stringent conditions to a coding nucleic acid identified as described herein, nucleic acids comprising nucleotide sequences which hybridize under stringent conditions to a fragment comprising at least 10, 15, 20, 25, 30, 35, 40, 50, 75, 100, 150, 200, 300, 400, or 500 consecutive nucleotides of one of the coding nucleic acids identified as described herein, nucleic acids comprising nucleotide sequences which hybridize under stringent conditions to a nucleic acid complementary to one of the coding nucliec acids identified as described herein, nucleic acids comprising nucleotide sequences which hybridize under stringent conditions to a fragment comprising at least 10, 15, 20, 25, 30, 35, 40, 50, 75, 100, 150, 200, 300, 400, or 500 consecutive nucleotides of the sequence complementary to one of the coding nucleic acids identified as described herein.

For example, the libraries may be screened to identify homologous coding nucleic acids or homologous antisense nucleic acids comprising nucleotide sequences which hybridize under moderate conditions to an antisense nucleic acid identified as described herein, nucleic acids comprising nucleotide sequences which hybridize under moderate conditions to a fragment comprising at least 10, 15, 20, 25, 30, 35, 40, 50, 75, 100, 150, 200, 300, 400, or 500 consecutive nucleotides of one of the antisense nucleic acids identified as described herein, nucleic acids comprising nucleotide sequences which hybridize under moderate conditions to a nucleic acid complementary to one of the antisense nucleic acids identified as described herein, nucleic acids comprising nucleotide sequences which hybridize under moderate conditions to a fragment comprising at least 10, 15, 20, 25, 30, 35, 40, 50, 75, 100, 150, 200, 300, 400, or 500 consecutive nucleotides of the sequence complementary to one of the antisense nucleic acids identified as described herein, nucleic acids comprising nucleotide sequences which hybridize under moderate conditions to a coding nucleic acid identified as described herein, nucleic acids comprising nucleotide sequences which hybridize under moderate conditions to a fragment comprising at least 10, 15, 20, 25, 30, 35, 40, 50, 75, 100, 150, 200, 300, 400, or 500 consecutive nucleotides of one of the coding nucleic acids identified as described herein, nucleic acids comprising nucleotide sequences which hybridize under moderate conditions to a nucleic acid complementary to one of the coding nucliec acids identified as described herein, nucleic acids comprising nucleotide sequences which hybridize under moderate conditions to a fragment comprising at least 10, 15, 20, 25, 30, 35, 40, 50, 75, 100, 150, 200, 300, 400, or 500 consecutive nucleotides of the sequence complementary to one of the coding nucleic acids identified as described herein.

The homologous coding nucleic acids, homologous antisense nucleic acids or homologous polypeptides identified as above can then be used as targets or tools for the identification of new, antimicrobial compounds using methods such as those described herein. In some embodiments, the homologous coding nucleic acids, homologous antisense nucleic acids, or homologous polypeptides may be used to identify compounds with activity against more than one microorganism.

For example, the preceding methods may be used to isolate homologous coding nucleic acids or homologous antisense nucleic acids comprising a nucleotide sequence with at least 97%, at least 95%, at least 90%, at least 85%, at least 80%, or at least 70% nucleotide sequence identity to a nucleotide identified as described herein, fragments comprising at least 10, 15, 20, 25, 30, 35, 40, 50, 75, 100, 150, 200, 300, 400, or 500 consecutive nucleotides thereof, and the sequences complementary thereto. Identity may be measured using BLASTN version 2.0 with the default parameters. (Altschul, S. F. et al. Gapped BLAST and PSI-BLAST: A New Generation of Protein Database Search Programs, Nucleic Acid Res. 25: 3389-3402 (1997), the disclosure of which is incorporated herein by reference in its entirety). For example, the homologous polynucleotides may comprise a coding sequence which is a naturally occurring allelic variant of one of the coding sequences identified as described herein. Such allelic variants may have a substitution, deletion or addition of one or more nucleotides when compared to the nucleic acids identified as described herein or the nucleotide sequences complementary thereto.

Additionally, the above procedures may be used to isolate homologous coding nucleic acids which encode polypeptides having at least 99%, 95%, at least 90%, at least 85%, at least 80%, at least 70%, at least 60%, at least 50%, at least 40% or at least 25% amino acid identity or similarity to a polypeptide encoded by one of the coding nucleic acids identified as described herein or to a polypeptpide whose expression is inhibited by one of the stabilized antisense nucleic acids identified as described herein or fragments comprising at least 5, 10, 15, 20, 25, 30, 35, 40, 50, 75, 100, or 150 consecutive amino acids thereof as determined using the FASTA version 3.0t78 algorithm with the default parameters. Alternatively, protein identity or similarity may be identified using BLASTP with the default parameters, BLASTX with the default parameters, or TBLASTN with the default parameters. (Altschul, S. F. et al. Gapped BLAST and PSI-BLAST: A New Generation of Protein Database Search Programs, Nucleic Acid Res. 25: 3389-3402 (1997), the disclosure of which is incorporated herein by reference in its entirety).

Alternatively, homologous coding nucleic acids, homologous antisense nucleic acids or homologous polypeptides may be identified by searching a database to identify sequences having a desired level of nucleotide or amino acid sequence homology to a nucleic acid or polypeptide involved in proliferation or an antisense nucleic acid to a nucleic acid involved in microbial proliferation. A variety of such databases are available to those skilled in the art, including GenBank and GenSeq. In some embodiments, the databases are screened to identify nucleic acids with at least 97%, at least 95%, at least 90%, at least 85%, at least 80%, or at least 70% nucleotide sequence identity to a nucleic acid required for proliferation, an antisense nucleic acid which inhibits proliferation, or a portion of a nucleic acid required for proliferation or a portion of an antisense nucleic acid which inhibits proliferation. For example, homologous coding sequences may be identified by using a database to identify nucleic acids homologous to one of the antisense nucleic acids identified as described herein, homologous to fragments comprising at least 10, 15, 20, 25, 30, 35, 40, 50, 75, 100, 150, 200, 300, 400, or 500 consecutive nucleotides thereof, nucleic acids homologous to one of the coding antisense nucleic acids identified as described herein, homologous to fragments comprising at least 10, 15, 20, 25, 30, 35, 40, 50, 75, 100, 150, 200, 300, 400, or 500 consecutive nucleotides thereof or nucleic acids homologous to the sequences complementary to any of the preceding nucleic acids. In other embodiments, the databases are screened to identify polypeptides having at least 99%, 95%, at least 90%, at least 85%, at least 80%, at least 70%, at least 60%, at least 50%, at least 40% or at least 25% amino acid sequence identity or similarity to a polypeptide involved in proliferation or a portion thereof. For example, the database may be screened to identify polypeptides homologous to a polypeptide comprising encoded by one of the coding nucleic acids identified as described herein, a polypeptide whose expression is inhibited by one of the stabilized antisense nucleic acids identified as described herein or homologous to fragments comprising at least 5, 10, 15, 20, 25, 30, 35, 40, 50, 75, 100, or 150 consecutive amino acids of any of the preceding polypeptides. In some embodiments, the database may be screened to identify homologous coding nucleic acids, homologous antisense nucleic acids or homologous polypeptides from cells or microorganisms other than the species from which they were obtained. For example, the database may be screened to identify homologous coding nucleic acids, homologous antisense nucleic acids or homologous polypeptides from microorganisms such as Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species.

In another embodiment, gene expression arrays and microarrays can be employed. Gene expression arrays are high density arrays of DNA samples deposited at specific locations on a glass chip, nylon membrane, or the like. Such arrays can be used by researchers to quantify relative gene expression under different conditions. Gene expression arrays are used by researchers to help identify optimal drug targets, profile new compounds, and determine disease pathways. An example of this technology is found in U.S. Pat. No. 5,807,522, which is hereby incorporated by reference.

It is possible to study the expression of all genes in the genome of a particular microbial organism using a single array. For example, the arrays may consist of 12×24 cm nylon filters containing PCR products corresponding to ORFs of coding nucleic acids identified as described herein. Ten nanograms of each PCR product are spotted every 1.5 mm on the filter. Single stranded labeled cDNAs are prepared for hybridization to the array (no second strand synthesis or amplification step is done) and placed in contact with the filter. Thus the labeled cDNAs are of “antisense” orientation. Quantitative analysis is done using a phosphorimager.

Hybridization of cDNA made from a sample of total cell mRNA to such an array followed by detection of binding by one or more of various techniques known to those in the art results in a signal at each location on the array to which cDNA hybridized. The intensity of the hybridization signal obtained at each location in the array thus reflects the amount of mRNA for that specific gene that was present in the sample. Comparing the results obtained for mRNA isolated from cells grown under different conditions thus allows for a comparison of the relative amount of expression of each individual gene during growth under the different conditions.

Gene expression arrays may be used to analyze the total mRNA expression pattern at various time points after induction of a stabilized antisense nucleic acid, which is flanked on each end by at least one stem-loop structure, complementary to a proliferation-required gene. Analysis of the expression pattern indicated by hybridization to the array provides information on other genes whose expression is influenced by stabilized antisense expression. For example, if the stabilized antisense is complementary to a gene for ribosomal protein L7/L12 in the 50S subunit, levels of other mRNAs may be observed to increase, decrease or stay the same following expression of the stabilized antisense to the L7/L12 gene. If the stabilized antisense is complementary to a different 50S subunit ribosomal protein mRNA (e.g. L25), a different mRNA expression pattern may result. Thus, the mRNA expression pattern observed following expression of a stabilized antisense nucleic acid comprising a nucleotide sequence complementary to a proliferation required gene may identify other proliferation-required nucleic acids. In addition, the mRNA expression patterns observed when the bacteria are exposed to candidate drug compounds or known antibiotics may be compared to those observed with stabilized antisense nucleic acids comprising a nucleotide sequence complementary to a proliferation-required nucleic acid. If the mRNA expression pattern observed with the candidate drug compound is similar to that observed with the stabilized antisense nucleic acid, the drug compound may be a promising therapeutic candidate. Thus, the assay would be useful in assisting in the selection of promising candidate drug compounds for use in drug development.

In cases where the source of nucleic acid deposited on the array and the source of the nucleic acid being hybridized to the array are from two different cells or microorganisms, gene expression arrays can identify homologous nucleic acids in the two cells or microorganisms.

Stabilized antisense nucleic acids complementary to specific proliferation-required genes flanked on each end by at least one stem-loop structure can also be used in cell-based assays to increase the sensitivity of cells to test compounds that have potential antibiotic activity. In one embodiment of the current invention, cell sensitivity to potential antibiotic compounds is increased by transcribing a specific antisense molecule or fragment thereof from a vector capable of producing stabilized transcripts in which the antisense molecule is flanked on each end by at least one stem-loop structure. Cell sensitivity can be further increased by transcribing the stabilized antisense RNA in a host having reduced ability to degrade RNA. Transcription of the stabilized antisense RNA reduces the expression of the complementary gene thereby causing the cell to display an increased sensitivity to compounds that effect processes in which the proliferation-required gene may be involved. Cells sensitized by this method are then contacted with one or more test compounds in order to determine which compounds cause a further reduction in cell proliferation. Compounds that normally would show little or no inhibitory effect on the proliferation of an unsensitized or undersensitized organism may inhibit the proliferation of organisms that have increased sensitivity to the expressed antisense molecules. These embodiments are particularly important given the rise of drug resistant bacteria.

The number of bacterial species that are becoming resistant to existing antibiotics is growing. A partial list of these microorganisms includes: Escherichia spp., such as E. coli, Enterococcus spp, such as E. faecalis; Pseudomonas spp., such as P. aeruginosa, Clostridium spp., such as C. botulinum, Haemophilus spp., such as H. influenzae, Enterobacter spp., such as E. cloacae, Vibrio spp., such as V. cholera; Moraxala spp., such as M. catarrhalis; Streptococcus spp., such as S. pneumoniae, Neisseria spp., such as N. gonorrhoeae; Mycoplasma spp., such as Mycoplasma pneumoniae; Salmonella typhimurium; Helicobacter pylori; Escherichia coli; and Mycobacterium tuberculosis. In some embodiments of the current invention, genes that have been identified as required for proliferation through the use of stabilized antisense expression can be used to identify homologous coding nucleic acids required for proliferation from these and other organisms. Other organisms include but are not limited to Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

Antisense nucleic acids complementary to genes required for the proliferation of a host organism from which the antisense molecules were originally obtained which are flanked on each end by at least one stem-loop structure may be used to identify homologous antisense nucleic acids and the coding nucleic acids complementary thereto from cells or microorganisms other than the original host organism, to inhibit the proliferation of cells or microorganisms other than the original host organism by inhibiting the activity or reducing the amount of the identified homologous coding nucleic acid or homologous polypeptide in the cell or microorganism other than the original host organism, or to identify compounds which inhibit the growth of cells or microorganisms other than the original host organism as described below. For example, antisense nucleic acids complementary to proliferation-required genes from original host organism which are flanked on each end by at least one stem-loop structure may be used to identify compounds which inhibit the growth of Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis and any species falling within the genera of any of the above species.

In one embodiment of the present invention, antisense nucleic acids complementary to the sequences identified as required for proliferation or portions thereof are transferred to vectors capable of replicating and producing stabilized transcripts in which the antisense nucleic acids are flanked on each end by at least one stem-loop structure within a species other than the species from which the proliferation-required sequences were obtained. For example, the vector may be capable of replicating and producing stabilized transcripts in Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species. In some embodiments of the present invention, the vector may be functional in gram-negative organisms. In other embodiments, the species other than the species from which the original proliferation-required sequences were obtained is a strain that has a reduced ability to degrade RNA.

As would be appreciated by one of ordinary skill in the art, the expression vectors may contain certain elements that are species specific. These elements can include promoter sequences, operator sequences, repressor genes, origins of replication, termination sequences, and others. To use the antisense nucleic acids, one of ordinary skill in the art would know to use standard molecular biology techniques to isolate vectors containing the nucleotide sequences of interest from cultured bacterial cells, isolate and purify those sequences, and subclone those sequences into a vector that is adapted for use in the species to be screened. To adapt a vector for the production of stabilized antisense transcripts, one of ordinary skill in the art would follow the teachings disclosed herein.

Vectors for a variety of other species, which can be adapted for expression of stabilized antisense transcripts, are known in the art. For example, numerous vectors which function in E. coli are known in the art. Also, Pla et al. have reported an expression vector that is functional in a number of relevant hosts including: Salmonella typhimurium, Pseudomonas putida, and Pseudomonas aeruginosa (J. Bacteriol. 172(8):4448-55 (1990)). Brunschwig and Darzins (Gene 111:35-4 (1992), the disclosure of which is incorporated herein by reference in its entirety) described a shuttle expression vector for Pseudomonas aeruginosa.

Following the subcloning of the antisense nucleic acids, which are complementary to proliferation-required sequences or portions thereof from the host organism from which the stabilized antisense nucleic acids were originally obtained, into a vector capable of replicating and producing stabilized transcripts in a second cell or microorganism of interest (i.e. a cell or microorganism other than the one from which the identified nucleic acids were obtained), the stabilized antisense nucleic acids are conditionally transcribed to test for bacterial growth inhibition. The coding sequences complementary to the antisense nucleic acids from the original host that, when transcribed, inhibit growth of the second cell or microorganism are compared to the known genomic sequence of the second cell or microorganism to identify the homologous gene from the second organism. If the homologous sequence from the second cell or microorganism is not known, it may be identified and isolated by hybridization to the proliferation-required sequence of interest from the original host organism or by amplification using PCR primers based on the proliferation-required nucleotide sequence of interest. In this way, nucleotide sequences which may be required for the proliferation of the second cell or microorganism may be identified. For example, the second microorganism may be Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species. In some embodiments of the present invention, the second microorganism is a gram-negative organism.

The homologous nucleic acid sequences from the second cell or microorganism which are identified as described above may then be operably linked to a promoter, such as an inducible promoter, in an antisense orientation and introduced into the second cell or microorganism. The techniques described herein for identifying genes required for proliferation may thus be employed to determine whether the identified nucleotide sequences from a second cell or microorganism inhibit the proliferation of the second cell or microorganism. For example, the second microorganism may be Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species. In some embodiments of the present invention, the second microorganism may be a gram-negative organism. If the stabilized antisense nucleic acid inhibits the proliferation of the second cell or microorganism, a cell-based assay, such as the one described herein, can be used to test and identify candidate antibiotic compounds.

In another embodiment of the present invention, screening of candidate antibiotic compounds can be performed directly by using the antisense molecule isolated from the original host organism. In this embodiment, a stabilized antisense nucleic acid comprising a nucleic acid complementary to the proliferation-required sequences from the original host organism or a portion thereof is transcribed in the second host from a vector capable of replicating and producing stabilized transcripts in the second host. If the stabilized antisense molecule is transcribed so as to sufficiently alter the level or activity of a nucleic acid required for proliferation of the second host, the second host may be used directly in a cell-based assay, such as those described herein, to identify candidate antibiotic compounds.

Stabilized antisense nucleic acids can also be used to identify the pathway on which a proliferation-required gene or gene product lies. In one embodiment of the present invention, an antisense nucleic acid that is complementary to a proliferation-required gene is provided to a test cell having a reduced ability to degrade RNA by using an antisense expression vector capable of producing stabilized transcripts. For example, the vector may be capable of replicating and producing stabilized transcripts in Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species. In some embodiments of the present invention, the vector may be functional in gram-negative organisms. The test cell is then contacted with a compound that causes inhibition of proliferation wherein the pathway on which this compound acts is known. If the test cell is more sensitive to the compound than a cell that has not been induced to express the antisense nucleic acid (for example, the test cell has a significantly lower IC₅₀ to the compound than the non-induced cell), the gene lies on the biological pathway on which the compound acts.

Stabilized antisense molecules can also be used to identify the pathway on which an antibiotic compound exerts its effect. In one embodiment of the current invention, an antisense nucleic acid that is complementary to a proliferation-required gene that is known to lie on a particular biological pathway is provided to a cell having a reduced ability to degrade RNA by using an antisense expression vector capable of producing stabilized transcripts. For example, the vector may be capable of replicating and producing stabilized transcripts in Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species. In some embodiments of the present invention, the vector may be functional in gram-negative organisms. The cell is then contacted with a test compound. If the cell is more sensitive to the compound than a cell that has not been induced to express the antisense nucleic acid (for example, the cell has a significantly lower IC₅₀ to the compound than the non-induced cell), the compound acts on the biological pathway on which the proliferation-required gene lies.

In still another embodiment, the stabilized antisense nucleic acids identified as described herein that inhibit bacterial growth or proliferation can be used as antisense therapeutics for killing bacteria. The stabilized antisense sequences can be complementary to one of the coding nucleotide sequences identified as described herein, homologous nucleotide sequences, or portions thereof. Alternatively, stabilized antisense therapeutics can be complementary to operons in which proliferation-required genes reside (i.e. the stabilized antisense nucleic acid may hybridize to a nucleotide sequence of any gene in the operon in which the proliferation-required genes reside). Further, stabilized antisense therapeutics can be complementary to a proliferation-required gene or portion thereof with or without adjacent noncoding sequences, an intragenic sequence (i.e. a sequence within a gene), an intergenic sequence (i.e. a sequence between genes), a sequence spanning at least a portion of two or more genes, a 5′ noncoding region or a 3′ noncoding region located upstream or downstream from the actual sequence that is required for bacterial proliferation or an operon containing a proliferation-required gene.

In addition to therapeutic applications, the present invention encompasses the use of stabilized nucleic acids complementary to nucleic acids required for proliferation as diagnostic tools. For example, stabilized nucleic acid probes comprising nucleotide sequences complementary to proliferation-required sequences that are specific for particular species of cells or microorganisms can be used as probes to identify particular microorganism species or cells in clinical specimens. This utility provides a rapid and dependable method by which to identify the causative agent or agents of a bacterial infection. This utility would provide clinicians the ability to accurately identify the species responsible for the infection and administer a compound effective against it. In an extension of this utility, antibodies generated against proteins translated from mRNA transcribed from proliferation-required sequences can also be used to screen for specific cells or microorganisms that produce such proteins in a species-specific manner.

The following examples teach methods of stabilizing antisense nucleic acids for use in the identification of proliferation-required genes and their corresponding operons, the identification of antibiotics which are active against organisms having reduced expression of proliferation-required genes, the identification of the biological pathway on which a proliferation-required gene or gene product lies and the identification of the biological pathway on which an antibiotic acts. Vectors for the expression of stabilized nucleic acid transcripts are also taught. These examples are illustrative only and are not intended to limit the scope of the present invention.

### EXAMPLES

The Stability of Antisense RNAs Affecting Cell Proliferation

Antisense polynucleotides are nucleic acids which are complementary, at least in part, to the coding strand of a gene. Antisense RNA may interact with sense mRNA that is transcribed from the corresponding gene so as to reduce or even eliminate protein production from the sense mRNA. In cases where the sense mRNA encodes a protein required for the proliferation, bacterial cells containing an activated expression vector that contains an antisense RNA fail to grow or grow at a substantially reduced rate. In general, the more of this antisense RNA that is present in the cell, the greater the reduction in the proliferation ability of the cell. When antisense molecules are produced in a cell through the use of an expression vector, the abundance of antisense RNA can be increased both by causing a greater number of transcripts to be produced from the vector (e.g., increasing the copy number of the expression vector containing the antisense polynucleotide, using a promoter having increased strength, and decreasing the repression of inducible promoters) and by increasing the stability of the transcribed antisense RNA by protecting it from degradation.

One method of protecting antisense RNA molecules from degradation is to utilize improved expression vectors that are designed to produce transcripts flanked at each end by at least one stem-loop structure. Another method is to express the antisense RNA in mutant host strains that have reduced ability to degrade RNA. The improved vectors can then be used in conjunction with the appropriate mutant host strains to transcribe stabilized antisense nucleic acids, some of which are complementary to proliferation-required nucleic acid sequences. The use of stabilized antisense nucleic acids increases the lifetime of the antisense RNAs and thus increases the potential for inhibition of the expression of proliferation-required genes. Accordingly, stabilized antisense RNA expression can be used to improve the efficacy of gene and drug discovery methods that are based on the inhibition of proliferation-required genes, such as the methods described herein. Such an improvement in efficacy translates into the discovery of a greater number and variety of proliferation-required genes and drugs which effect those genes.

The following Examples describe the construction of vectors that are useful for expressing stabilized antisense RNA as well as methods for expressing stabilized antisense nucleic acids in host cells having reduced ability to degrade RNA. The Examples also describe methods for using stabilized antisense nucleic acids to identify proliferation-required genes, operons containing the proliferation-required genes, pathways on which the proliferation-required genes lie, compounds having antibiotic properties, pathways on which antibiotic compounds act, and proliferation-required genes in organisms heterologous to the organism from which the antisense was obtained.

### Example 1

### Construction of an Expression Vector Lacking RNase E Recognition Sites in the Transcribed Region Downstream of the Inducible Promoter

The following describes the construction of an expression vector which lacks recognition sites for RNase E in the transcribed region downstream of the inducible promoter. The expression vector pLEX5BA (described by, Krause et al., J. Mol. Biol. 274:365-380 (1997), for a general description of pLEX vectors, see Diederich et al., BioTechniques 16:916-923 (1994), the disclosures of which are incorporated herein by reference in their entirety) was modified to remove two RNase E recognition sites within its transcriptional terminator region.

The transcriptional terminator region of pLEX5BA is located downstream of the multiple cloning site (MCS) on an approximately 464 base pair HindIII to ClaI restriction endonuclease fragment. This region contains two endogenous RNase E recognition sites that flank the 5S rRNA gene which immediately precedes the tandem rrnBt1t2 terminator sequences. These RNase E sites are important in the processing of stable 5S rRNA.

A PCR based strategy was use to delete the entire nucleic acid region encoding the 5S rRNA gene and the two RNase E recognition sites. Two oligonucleotides were synthesized (Integrated DNA Technologies, Inc., Coralville, Iowa; SEQ ID NOs.: 1 & 2) and used as primers in PCR reactions to specifically amplify a 353 bp portion of pLEX5BA which includes the tandem rrnBt1t2 terminator sequences but lacks the two RNase E sites.

| | |
|---|---|
| CCGGAAGCTTATAAAACGAAAGGCTCAGTCGA | SEQ ID NO.:1 |
| AGGTGCCTCACTGATTAAGC | SEQ ID NO.:2 |

The primer represented by SEQ ID NO.:1 is identical to the 5′ end of the T1 terminator except that it includes 10 nucleotides at its 5′ end which contain a HindIII recognition site (underlined nucleotide indicate the HindIII recognition site). After amplification, the PCR fragment was digested with HindIII and ClaI overnight at 37° C. in a 15 μl total volume then subjected to electrophoresis on a 2% agarose gel thereby producing a 273 bp HindIII-ClaI fragment which was purified from the gel using a commercially available kit (QiagenGel Extraction Kit, Qiagen Corp.) according to the manufacturer's instructions.

Approximately 12 μg of pLEX5BA was digested with HindIII and ClaI at 37° C. overnight, then subjected to electrophoresis on a 1.5% agarose gel. The fragment was isolated from the gel using the commercially available kit described above. The gel purified 273 bp fragment was then ligated into the HindIII and ClaI sites of digested pLEX5BA vector at 15° C. overnight using T4 DNA ligase (New England BioLabs, Beverly, Mass.). A portion of the ligation mixture (˜50 ng of DNA) was used to transform competent XL-1 Blue cells (Stratagene, La Jolla, Calif.) and 1 μl, 10 μl, or 100 μl of the transformation mixture was plated onto L-broth plates supplemented with 100 μg/ml of carbenicillin. Isolated carbenicillin-resistant transformants were picked, and streaked to obtain single colony isolates. Plasmid DNA was then purified from representative single colony transformants. The presence of the insert in each construct was confirmed by PCR amplification of the cloned region using oligonucleotide primers flanking the insert site. Clones having the appropriate size inserts were then sequenced to ensure that the desired modifications had been made. The nucleotide sequences were determined using plasmid DNA isolated using QIAPREP (Qiagen, Valencia, Calif.) and methods supplied by the manufacturer.

The resulting plasmid, pLEX5BAΔ5S, was further modified along with the parent vector pLEX5BA, to allow for the introduction of a 5′-stem-loop structure in the resulting antisense RNA.

### Example 2

### Engineering an Expression Vector to Contain a Stem-loop-encoding Sequence Beginning at the Site of Transcription Initiation

The following describes the construction of expression vectors that include a stem-loop-encoding nucleic acid sequence having its 5′ end at the site of transcription initiation. The vectors pLEX5BA and pLEX5BAΔ5S both contain an IPTG-inducible T7 promoter-operator P_{A1-03/04} (described by, Lanzer and Bujard, Proc. Natl. Acad. Sci. USA 85:8973-8977 (1988), the disclosure of which is incorporated herein by reference in its entirety) for inducible expression of heterologous nucleic acids. The two lacI operators are located just upstream of the MCS. The first operator is located between the −10 and −35 regions of the promoter whereas the second operator overlaps the site of transcription initiation.

Both pLEX5BA and pLEX5BAΔ5S were modified by removing a MunI to HindIII restriction fragment containing the second lacI operator and the majority of the MCS then replacing that fragment with a synthetic polynucleotide sequence corresponding to an altered lacI operator sequence and a truncated MCS. This synthetic polynucleotide was constructed so as to reflect a single point mutation in the sequence of the second lacI operator thereby creating a perfect inverted repeat. Because the 5′ end of the inverted repeat corresponds to the transcription start site, the first nucleotide of the transcript initiated from the P_{A1-03/04} promoter is included in the basal most portion of the stem. Accordingly, all transcripts produced from such vectors have a flush, double stranded 5′ end.

To prepare the vectors for insertion of the synthetic operator/MCS sequence, approximately 4 μg of purified pLEX5BA and pLEX5BAΔ5S DNA were individually mixed with 20 units each of the restriction endonucleases MunI and HindIII in 60 μl total volume then incubated at 37° C. for 2 hours. After electrophoresis on a 1% agarose gel, the linearized vectors were isolated using a commercially available kit (Qiagen Gel Extraction Kit, Qiagen Corp.) according to the manufascturer's instructions.

To generate the modified operator/MCS sequence, two 5′-phosphorylated, complementary oligonucleotides were synthesized (Integrated DNA Technologies, Inc., Coralville, Iowa; SEQ ID NOs.: 3 & 4) containing a mutant lacI operator sequence, a portion of the MCS and either a MunI or HindIII 5′-overhang ends.

SEQ ID NO.:3

5′-AATTGTGAGCGGATCACAATTGAATTCCCGGGA-3′

SEQ ID NO.:4

5′-AGCTTCCCGGGAATTCAATTGTGATCCGCTCAC-3′

The underlined nucleotides comprise the 5′-overhang of the MunI restriction endonuclease site whereas the italicized nucleotides correspond to the 5′-overhang of a HindIII restriction endonuclease site. The first underlined adenine nucleotide is the first nucleotide of the resulting transcript and the nucleotide shown in bold type is the substituted nucleotide of the lacI operator sequence (i.e., a change of A to C). The only restriction enzyme recognition sites retained from the original MCS are EcoRI (GAATTC) and SmaI (CCCGGG). The modified lacI operator portion of this sequence (AATTGTGAGCGGATCACAATT) (Nucleotides 1 to 21 of SEQ ID NO.: 3) contains a perfect 8 base pair inverted repeat interposed by a loop of 5 nucleotides.

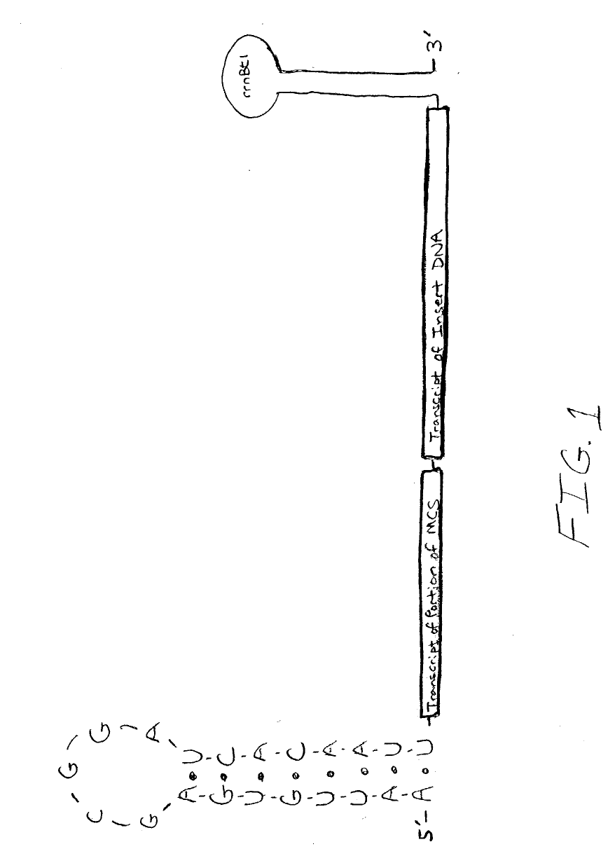

The secondary structure formed by this inverted repeat and its free energy of formation was determined using mfold version 3.0 (available at http://bioinfo.math.rpi.edu/˜zukerm/). FIG. 1 shows the structure of the most stable stem-loop that can be formed by the hydrogen bonding of the complementary residues of this region (SEQ ID NO.: 5). The predicted free energy of formation of this stem-loop structure is −7.2 kca/mol.

The double stranded operator/MCS sequence was created by combining an equimolar amount of SEQ ID NOs.: 3 and 4 in 100 μl of 10 mM Tris-HCl, pH 8.5, heating the mixture to 90° C. to denature any secondary structure, and allowing the complementary sequences to anneal by slowly cooling the solution to room temperature. The double stranded synthetic operator/MCS sequence was then ligated into the MunI and HindIII sites of digested pLEX5BA and pLEX5BAΔ5S vectors at 15° C. overnight using T4 DNA ligase (New England BioLabs). A portion of the ligation mixture (˜50 ng of DNA) was used to transform competent Top₁₀ Econ cells (Invitrogen, Carlsbad, Calif.) and 100 μl of the transformation mixture was plated onto L-broth plates supplemented with 100 μg/ml of carbenicillin. Isolated carbenicillin-resistant transformants were picked and streaked to obtain single colony isolates. Plasmid DNA was purified from representative single colony transformants.

The presence of the insert in each construct was confirmed by PCR amplification of the cloned region using oligonucleotide primers flanking the insert site. Clones having inserts of the appropriate size were then sequenced to ensure that the desired modifications had been made. Clones of pLEX5BA that were successfully engineered to contain the synthetic lacI/MCS insert were named pEPEC2 whereas the appropriate derivatives of pLEX5BAΔ5S were designated pEPEC3.

One of ordinary skill in the art will recognize that pLEX series plasmids as well as other expression vectors can be combined with a variety of polynucleotides having inverted repeat sequences to introduce one or more stem-loop structures into expressed transcripts at or near their 5′ ends. One of ordinary skill in the art will also recognize that expression vectors for use in organisms other than E. coli, as well as shuttle vectors for use between two or more different organisms, can be engineered to contain nucleic acid sequences which encode stem-loop structures that are transcribed, thereby producing RNA molecules that are stabilized in the host organisms.

### Example 3

### Improved Stability of Stem-Loop-Containing Transcripts that are Produced from Engineered Vectors

The following describes the ability a certain pEPEC series vectors (construction described above) to produce stable RNA transcripts in various strains of E. coli. The relative stability of transcripts produced by these vectors was shown by Northern blotting.

Vector RNAs were produced by separately transforming each of the pEPEC vectors and pLEX5BA into competent MG1655 and Top₁₀ Econ cells (Invitrogen, Carlsbad, Calif.). Transformed cells were grown in L-broth at 37° C. with shaking to early log phase. Transcription was then induced from the P_{A1-03/04} promoter by the addition of IPTG to the cell cultures. Immediately prior to the induction of transcription, a portion of the cell culture was withdrawn and combined with a half volume of a boiling hot buffer containing 1.5% sodium dodecylsulfate, 0.3 M sodium acetate, 30 mM EDTA, pH 7.0. This sample was boiled for 1 minute, cooled to room temperature, then stored on ice. After phenol and chloroform extractions, the cellular RNA was precipitated with ethanol and washed several times before use (for extraction and precipitation of nucleic acids see, Maniatis et al., Molecular Cloning: A Laboratory Manual, 2nd Ed., Cold Spring Harbor Laboratory Press, Cold Spring Harbor, N.Y. (1989), the disclosure of which is incorporated herein by reference). The RNA obtained from this sample was termed the preinduction control. Ten minutes after the IPTG induction, 500 μg/ml rifampicin (Sigma, St. Louis, Mo.) was added to the cultures to inhibit all subsequent transcription. At predetermined time points subsequent to rifampicin addition, a portion of each cell culture was withdrawn and total cellular RNA was isolated using the boiling SDS method as described above.

The relative stability of the RNA produced by each vector was determined by comparing the abundance vector transcripts that were isolated at each time point subsequent to the inhibition of transcription by rifampicin. Relative transcript abundance was determined by Northern blot analysis. To perform Northern blot analysis, the RNA samples were first subjected to electrophoresis on formaldehyde agarose gels. The RNA was then transferred from the gel to a nitrocellulose membrane (for formaldehyde gel preparation and RNA transfer see, Maniatis et al., (1989), the disclosure of which is incorporated herein by reference). After transfer, the RNA was fixed to the membrane by using a UV crosslinker (Stratagene, La Jolla, Calif.) as per manufascturer's instructions. Polynucleotide probes complementary to the vector RNA were made using the StarFire Oligonucleotide Labeling System (IDT Technologies, Coralville, Iowa) as per manufascturer's instructions. Each probe was purified by loading it in a 50 μl volume onto a freshly prepared Sephadex G25 spin column and then centrifuging for 4 minutes at 1100 g to recover the labeled probe. Hybridization was performed essentially as previously described (Maniatis et al., Molecular Cloning: A Laboratory Manual, 2nd Ed., Cold Spring Harbor Laboratory Press, Cold Spring Harbor, N.Y. (1989), the disclosure of which is incorporated herein by reference). After the final wash, the blot was placed in a Phosphorimager for 0.5 to 2 hours. The image was visualized using Imagequant® software (Molecular Dynamics, Sunnyvale, Calif.). Subsequent to phosphorimaging, the blot was used to produce an autoradiogram.

In E. coli strains having wildtype RNase E, it was expected that vectors containing recognition sites for this enzyme would produce transcripts that rapidly lose their stabilizing 3′ stem-loops due to site specific cleavage. Accordingly, vectors which encode such recognition sites would produce RNAs having essentially no protective 3′ stem-loop structures.

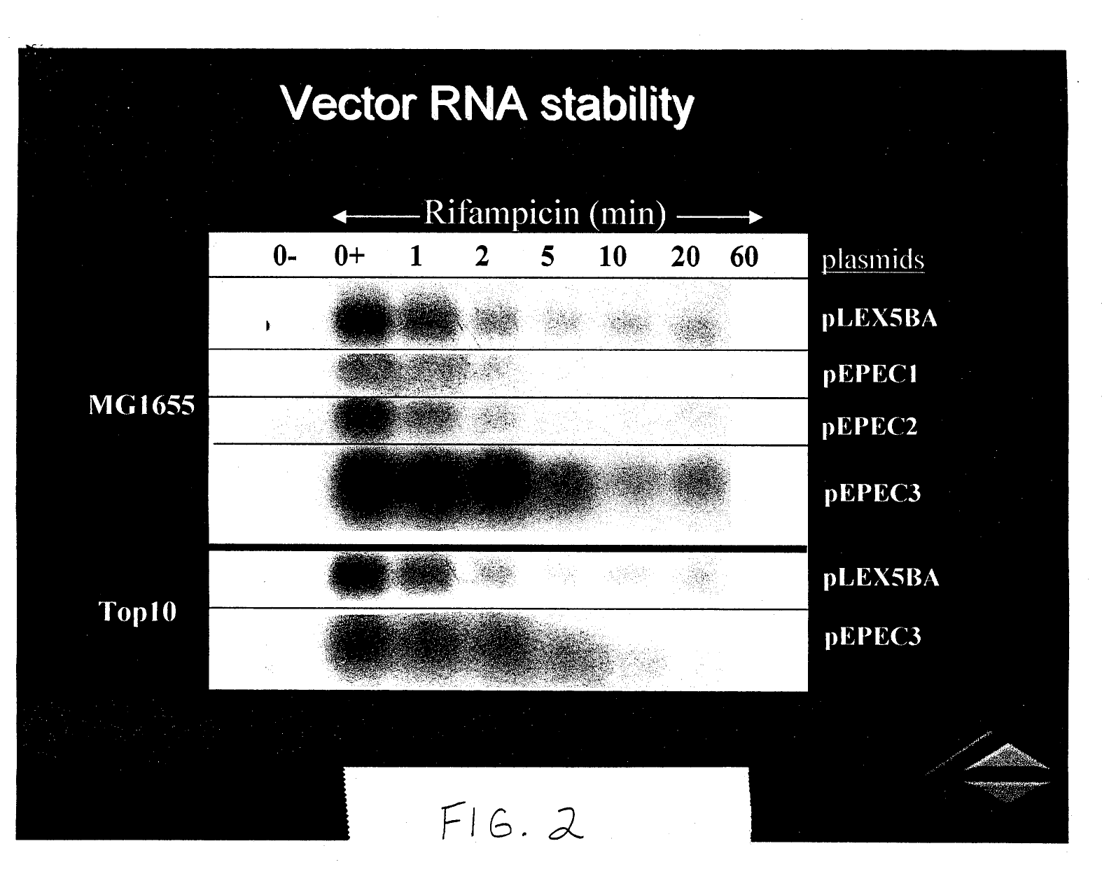

FIG. 2 shows the stability of stem-loop-containing RNA that has been transcribed from the three newly engineered vectors (pEPEC1, which is equivalent to pLEX5BAΔ5S, pEPEC2 and pEPEC3) as well as the parental vector pLEX5BA in two different strains of E. coli. Each of the expressed RNAs correspond only to transcribed vector sequence as no exogenous nucleic acids have been inserted into the multiple cloning sites. The size of the transcripts varies from 90 to 275 nt for the pEPEC vectors. The transcript produced by pLEX5BA is 333 nt. In MG1655 cells, transcripts produced by the parental vector pLEX5BA (unmodified) as well as those produced by pEPEC1 (3′ stem-loop and deletion of RNase E sites) and pEPEC2 (5′ stem-loop but no deletion of RNase E sites) are relatively unstable compared to pEPEC3 transcripts (5′ stem-loop and 3′ stem-loop having RNase E sites deleted). More specifically, FIG. 2 shows that no transcripts were produced by any of these vectors before induction with IPTG (0-time point). However, after IPTG induction and throughout the time course, the pEPEC3 transcript is present in greater abundance than those having only one or no stable stem-loop structures. Such results translate into an approximately 3- to 5-fold increase in stability of transcripts produced by pEPEC3 when compared to those produced by the other vectors. Similar results are seen for the stability of transcripts in Top 10 cells.

The result that the stem-loop structures at each end act cooperatively to increase transcript stability was unexpected. FIG. 2 shows that neither the introduction of a only a stable 3′ stem-loop structure (pEPEC1) nor the introduction of only a stable 5′ stem-loop structure (pEPEC2) has a significant effect on vector RNA stability. Accordingly, the increased stability resulting from the introduction of both features into the same transcript cannot be an additive effect. Only a synergistic or cooperative effect can explain these results.

It will be appreciated that transcript stability can be increased in other organisms by engineering appropriate expression vectors to produce RNA transcripts having both 5′ and 3′ stem-loop structures. In one embodiment of the present invention, an appropriately engineered expression vector is used to produce stabilized RNA in a gram-negative organism.

### Example 4

### Improvement of the Stability of Transcripts Produced by pEPEC3 in Cells Having Reduced Ability to Degrade RNA

The following describes improvements in the stability of the pEPEC3 transcript in E. coli mutants having reduced ability to degrade RNA. The relative stability of transcripts produced in these mutant strains was shown by Northern blotting.

pEPEC3 was transformed into competent a wildtype E. coli strain (MG1655), an E. coli mutant strain (DW97) having a mutation in the gene encoding RNase E (rne131), and an E. coli double mutant strain (DW98) having both the rne131 mutation and a mutation in pnp, a gene that encodes the 3′ to 5′ exoribonuclease polynucleotide phosphorylase. Transformed cells were grown in L-broth and transcription was induced with IPTG during early log phase. As described above, cell samples were taken immediately before induction and at specific times subsequent to the inhibition of transcription with rifampicin. RNA was isolated from each cell sample by using the boiling SDS method (described above), then subjected to electrophoresis on formaldehyde agarose gels. Relative transcript abundance was determined by Northern blots analysis (described above).

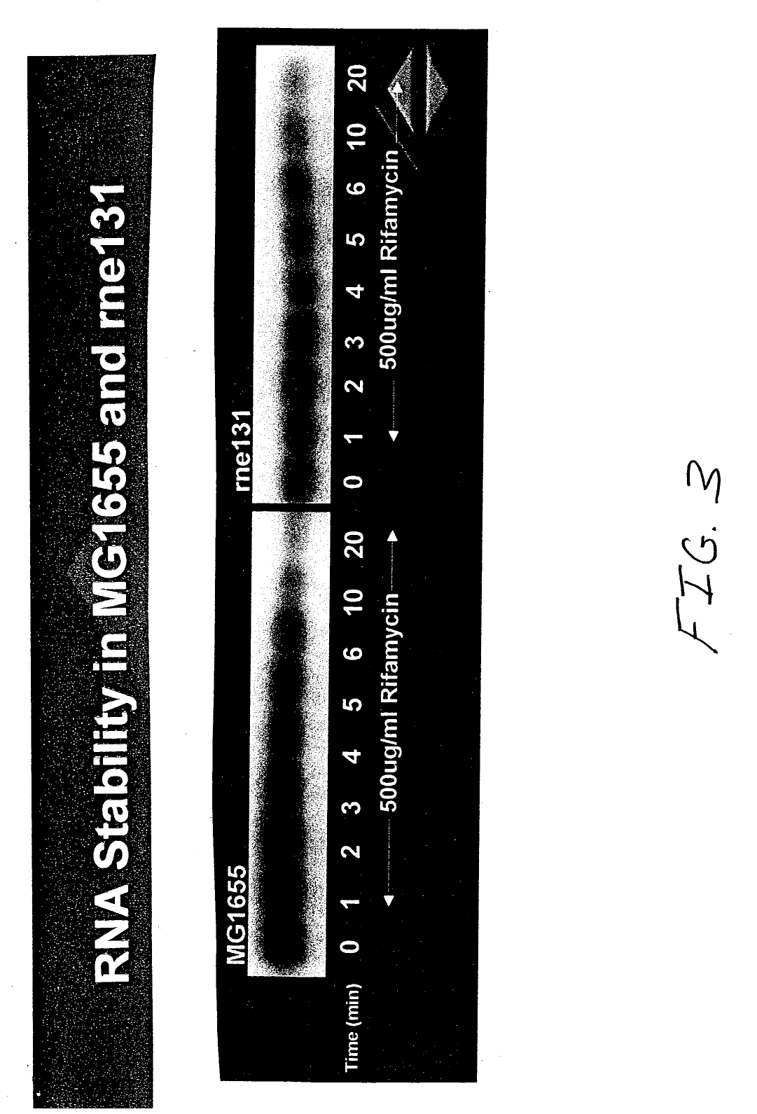

FIG. 3 shows that the expression of pEPEC3 RNA in a strain lacking RNase E activity results nearly a 2-fold increase in the half life of the vector RNA when compared to the RNA produced in wildtype cells. Similarly, when the stability of the RNA produced in the double mutant is compared to the stability of RNA produced in wildtype cells, the half life of the vector RNA in the mutant is about twice the half life of the RNA in wildtype cells. A simple linear regression analysis of the log of the percent RNA signal reduction over time indicates that the half life of a pEPEC3 transcript increases from 7 to 12 minutes when it is produced in the rne 131 mutant rather than wildtype E. coli.

### Example 5

### Increased Sensitivity to Stem-Loop-Stabilized Antisense Nucleic Acids that Inhibit Proliferation-required Genes in Cells Having Reduced Ability to Degrade RNA

To demonstrate the sensitizing effect of the in vivo production of stabilized antisense RNA to proliferation-required genes, three antisense nucleic acids previously identified to inhibit proliferation were subcloned into pEPEC3 then stably expressed in E. coli strains deficient in one or more enzymatic activity involved in RNA degradation. In general, the three antisense polynucleotides to proliferation-required genes were separately cloned into the MCS of pEPEC3, the vectors were introduced into different E. coli strains, transcription was induced and the change in the proliferation ability of each strain was measured. Because RNA expression was further stabilized in strains having reduced ability to degrade RNA, such mutants displayed increased sensitivity to proliferation-inhibiting antisense transcripts which manifested as a greater reduction in their proliferation ability.

Antisense nucleic acids to an E. coli glutaminyl tRNA synthetase (glnS) as well as rplL, rplJ and rplW were operably linked to the IPTG-inducible promoter of the pEPEC3 expression vector. The rplL, rplJ antisense polynucleotide is a nucleic acid fragment that contains a portion of each the rplL and the rplJ gene sequences. The rplL, rplJ and rplW genes encode ribosomal proteins L7/L12, L10 and L23, respectively. Expression constructs containing one of the above antisense polynucleotides were then separately transformed into the wildtype E. coli strain MG1655, the E. coli mutant strain DW97 which has a mutation in the gene encoding RNase E (rne131), the E. coli double mutant strain DW98 which has both the rne131 mutation and a mutation in pnp, a gene that encodes the 3′ to 5′ exoribonuclease polynucleotide phosphorylase, the E. coli double mutant strain DW99 which has both the RNase E (rne131) mutation and a mutation in pcnB, a gene that encodes a polyA RNA polymerase, or the E. coli strain SK5704 a triple mutant which has mutations in pnp -7, rne-1 and rnb-500, the gene that encodes RNase II.

The sensitivity of each E. coli strain to the antisense transcripts produced by pEPEC3 was measured by determining the ability of each strain to grow on selective L-broth agar plates having varying concentrations of IPTG. Overnight cultures of each strain were grown then 10, 10², 10³, 10⁴, 10⁵, 10⁶, 10⁷ and 10⁸ fold dilutions were prepared. Aliquots of from 0.5 to 3 μl of these dilutions were spotted on selective agar plates containing 0, 25, 50, 100, 150, 200, or 400 μM IPTG, however, aliquot volume was consistent between strains and over the range of IPTG concentrations. After overnight incubation at 37° C., the plates were compared to assess the sensitivity of the clones to IPTG. Specifically, the minimum concentration of cells (most dilute culture) that was capable of supporting cell growth on IPTG-containing solid medium was determined for each strain. This determination was also made for cultures plated on medium lacking IPTG. The log value for each minimum concentration was then calculated and used to represent the proliferation ability (sensitivity to the antisense transcript) of each strain at each IPTG concentration. The change in the proliferation ability of a mutant strain was measured by determining the difference between its proliferation ability value and that of MG1655. Because log values were used, a difference of one unit represents a 10-fold change in proliferation ability and accordingly, a 10-fold change in sensitivity to the antisense transcript.

Alternative methods of measuring growth are also contemplated. Examples of these methods include measurements of proteins, the expression of which is engineered into the cells being tested and can readily be measured. Examples of such proteins include green fluorescent protein (GFP) and various enzymes.

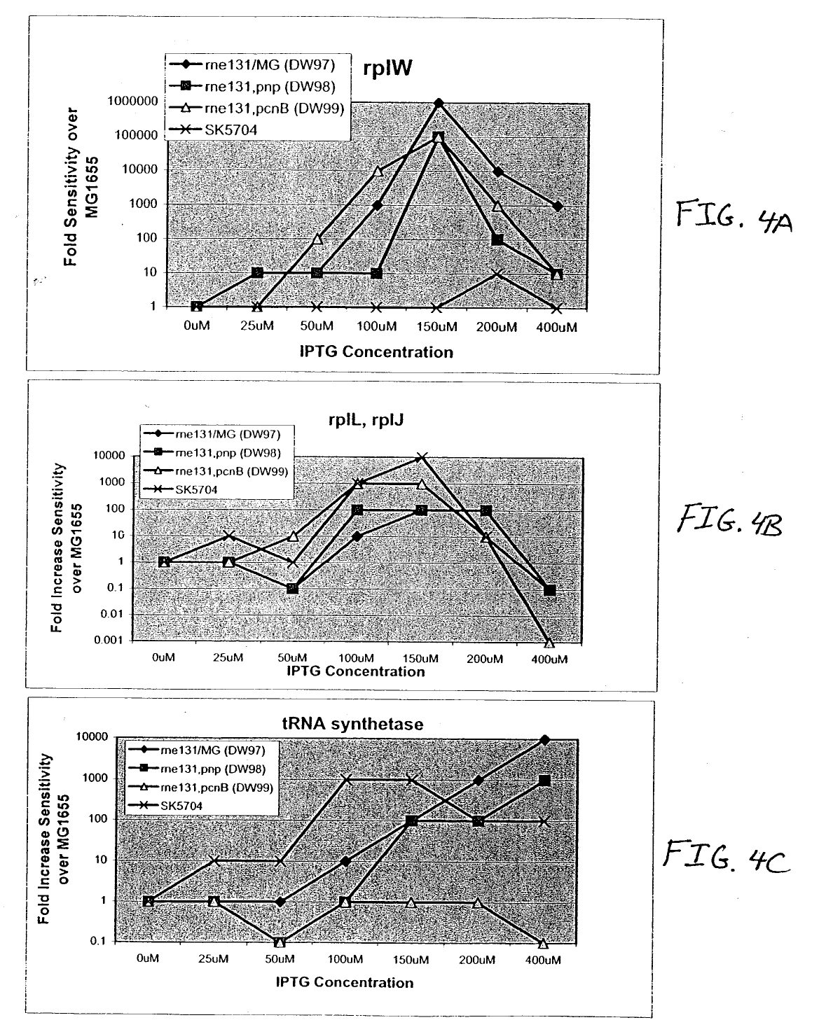

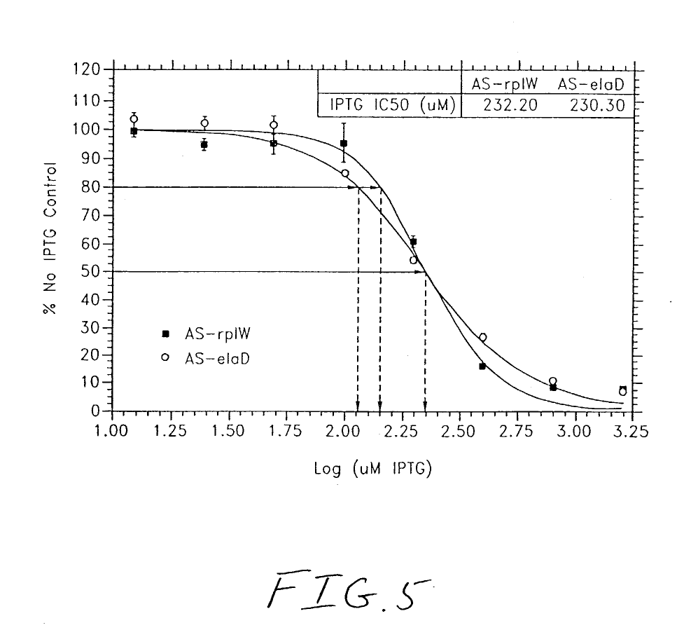

Merely having one or more mutations that reduce the cells' capability to degrade RNA was not sufficient to alter their rate of proliferation. FIGS. 4a-c each show that none of the mutant strains suffered a reduction in growth compared to MG1655 when IPTG was not added to the medium. This was true regardless of which pEPEC3 antisense construct that was introduced into the cells (compare FIGS. 4a-c).

FIG. 4a illustrates the increased sensitivity of the mutant E. coli strains to the rplW antisense nucleic acid-expressed by pEPEC3. At low concentrations of IPTG (25 to 50 μM) there was little difference in the proliferation ability of any of the mutant strains as compared to the wildtype. At increased IPTG levels (100 to 150 μM) both the single mutant (DW97) and the double mutants (DW98, DW99) showed a significant increase in sensitivity to the rplW antisense RNA. It was unexpected that the single mutant strain would show a greater increase in sensitivity to rplW antisense than the double mutants; however, compared to the MG1655 wildtype, DW97 displayed a 10⁶-fold decrease in proliferation ability, whereas both DW98 and DW99 showed only a 10⁵-fold decrease. At the highest IPTG concentrations (200-400 μM) the difference in sensitivity between the mutants and wildtype began to decrease. Unexpectedly, the proliferation ability of the triple mutant SK5704 was not significantly different from that of the wildtype over the entire range or IPTG concentrations.

FIG. 4b illustrates the increased sensitivity of the mutant E. coli strains to the rplL, rplJ antisense transcript expressed by pEPEC3. The curves shown here are similar in shape to those displayed in FIG. 4a in that there appears to be little difference in proliferation ability at low IPTG concentrations, a maximal difference at midlevel concentrations, and a decrease in the difference at the highest IPTG concentrations. For each mutant, the greatest increase in sensitization occurred at about 150 μM IPTG. The most sensitive of the strains was the triple mutant SK5704 which showed a 10⁴-fold decrease in proliferation ability. Unexpectedly, each of the mutants displayed decreased sensitivity to the rplL, rplJ antisense transcript when compared to MG1655 wildtypes at the highest IPTG concentration (400 μM). Of the four mutants, DW97 had the largest decrease in rplL, rplJ antisense sensitivity which was 10³-fold.

FIG. 4c illustrates the increased sensitivity of the mutant E. coli strains to an antisense nucleic acid expressed by pEPEC3 that corresponds to the sense strand of the gene encoding glnS which is required for proliferation. The response of SK5704 to the expression of this stabilized antisense was similar to its response to the expression of rplL, rplJ in FIG. 4b. The sensitivity of both DW97 and DW98 increased over nearly the entire range of IPTG concentrations with each strain displaying the greatest increase in sensitivity at 400 μM. Unexpectedly, the single mutant DW97 showed the greatest increase in sensitivity, which was 10⁴-fold, whereas the double mutant DW98 showed only 10³-fold increase. The proliferation ability of the double mutant DW99 was not significantly different from that of the wildtype over the entire range or IPTG concentrations.

It is clear that mutant microbial strains having reduced ability to degrade RNA are useful hosts in which to transcribe stabilized antisense nucleic acids.

It will be appreciated that antisense RNA stability may be enhanced in organisms other than E. coli. Expression vectors capable of replicating in hosts other than E. coli, including shuttle vectors, can be modified so as to produce stable antisense transcripts flanked on each end by stem-loop structures. Moreover, microorganisms other than E. coli include strains which have reduced ability to degrade RNA. Examples of such organisms are Psuedomonas aeruginosa and Klebsiella pneumoniae. Accordingly, an artisan of ordinary skill will recognize that the use of such strains in conjunction with appropriately modified expression vectors can increase the stability of transcripts in a wide range of host organisms.

The transcription of stabilized antisense RNAs can aid in the detection of novel genes required for proliferation in a wide range of microbes. Transcription of stabilized antisense RNAs increases the lifetime and thus the inhibitory effectiveness of antisense transcripts which may be short-lived, thereby resulting in identification of proliferation required genes which might not be detected with non-stabilized antisense RNAs. An example of the use of stabilized antisense transcripts in the discovery of proliferation required genes is provided below.

### Example 6

### Inhibition of Bacterial Proliferation after Induction of Stabilized Antisense Expression

Methods for the inhibition and subsequent identification of proliferation-required genes have been previously described (in U.S. Pat. No. 6,228,579, International Publication WO 00/44906 and International Publication WO 01/70955, the disclosures of which are incorporated by reference in their entirety). The procedure is described here as follows.

Random genomic fragments are cloned into an inducible expression vector designed to produce stabilized transcripts having at least one stem-loop structure flanking each end of the transcribed genomic sequence The genomic fragments are then assayed to determine their effect on cell growth. For example, derivatives of pLEX5BA (Krause et al., J. Mol. Biol. 274:365 (1997)) capable of producing stabilized RNA transcripts, such as those described in Examples 1 and 2, can be used.

Upon induction, the vector produces a stabilized RNA molecule corresponding to the subcloned genomic fragments. In those instances where the genomic fragments are in an antisense orientation with respect to the promoter, the transcript produced is complementary to at least a portion of an mRNA (messenger RNA) encoding a gene product such that they interact with sense mRNA produced from various genes and thereby decrease the translation efficiency or the level of the sense messenger RNA thus decreasing production of the protein encoded by these sense mRNA molecules. In cases where the sense mRNA encodes a protein required for proliferation, bacterial cells containing a vector from which transcription from the promoter has been induced fail to grow or grow at a substantially reduced rate. Additionally, in cases where the transcript produced is complementary to at least a portion of a non-translated RNA and where that non-translated RNA is required for proliferation, bacterial cells containing a vector from which transcription from the promoter has been induced also fail to grow or grow at a substantially reduced rate.

In one specific example, the effect of transcribing random genomic fragments on the proliferation ability of E. coli was determined. Random fragments of E. coli genomic DNA were generated by DNaseI digestion or sonication, filled in with T4 polymerase, and ligated into the SmaI site of pEPEC3. These ligation products were then transformed into electrocompetent E. coli strain XL1-Blue MRF (Stratagene) and transformants were plated on LB medium with carbenicillin at 100 μg/ml. Resulting colonies numbering 5×10⁵ or greater were scraped and combined, and were then subjected to plasmid purification.

The purified plasmid library was then re-transformed into electrocompetent E. coli. Resulting transformants were plated on LB agar with carbenicillin at 100 μg/ml in order to generate 100 to 150 platings at 500 colonies per plating. The colonies were subjected to robotic picking and arrayed into wells of 384 well culture dishes. Each well contained 100 μL of LB containing carbenicillin at 100 μg/ml. Inoculated 384 well dishes were incubated 16 hours at room temperature, and each well was robotically gridded onto solid LB containing carbenicillin at 100 μg/ml with or without 1 mM IPTG. Gridded plates were incubated 16 hours at 37 ° C., and then manually scored for arrayed colonies that were growth-compromised in the presence of IPTG.

Arrayed colonies that were growth-sensitive on medium containing 1 mM IPTG, yet were able to grow on similar medium lacking IPTG, were subjected to further growth sensitivity analysis. To study the effects of transcriptional induction in liquid medium, growth curves were carried out by back diluting cultures 1:200 into fresh media with or without 1 mM IPTG and measuring the OD₄₅₀ every 30 minutes (min). To study the effects of transcriptional induction on solid medium, 10², 10³, 10⁴, 10⁵, 10⁶, 10⁷ and 10⁸ fold dilutions of overnight cultures were prepared. Aliquots of from 0.5 to 3 μl of these dilutions were spotted on selective agar plates with or without 1 mM IPTG. After overnight incubation, the plates were compared to assess the sensitivity of the clones to IPTG. Plasmid DNA was recovered from the sensitive clones. Example 7 describes the nucleotide sequence determination of the clones that inhibited proliferation in E. coli.

It will be appreciated that a variety of alternative techniques can be used to obtain genomic DNA fragments for expression. Examples of such techniques include but are not limited to nebulization or other mechanical shearing methods and digestion with one or more restriction enzymes or other endonucleases. It will also be appreciated that vectors other than pEPEC3 can be used to transcribe the genomic DNA inserts. An artisan of ordinary skill will recognize that pEPEC3 can be modified to introduce features such as stop codons in all three reading frames downstream of the genomic DNA inserts to ensure that if the genomic DNA insert encodes a polypeptide (i.e. the insert is in the sense orientation rather than the antisense orientation or the insert is in the antisense orientation but contains a cryptic ORF) translation of the polypeptide will terminate shortly after the genomic insert.

One of ordinary skill in the art will recognize that expression vectors capable of replicating in hosts other than E. coli, including shuttle vectors, can also be modified so as to produce stem-loop-stabilized antisense transcripts.

It will also be appreciated that the above strategy for identifying proliferation-required genes and various methods based on this strategy, including but not limited to sensitizing an organism to certain chemical compounds by reducing its ability to proliferate, can be performed in a variety of hosts other than E. coli by inducing transcription of stabilized antisense nucleic acids from vectors capable of replicating in the host organism then monitoring its growth (see International Publication WO 01/70955, the disclosure of which is incorporated herein by reference in its entirety). Examples of such organisms include but are not limited to Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species. In one embodiment of the present invention, these organisms include gram-negative bacteria. In other embodiments, the host organism has a reduced ability to degrade RNA. In some embodiments the host organism is grown with vigorous shaking to ensure homogenous distribution throughout the growth medium and thus, accurate measurement of proliferation ability.

One of ordinary skill in the art will further appreciate that expression of stabilized antisense RNA from genomic fragments in a host other than E. coli can be induced from vectors that have been modified to produce stem-loop-stabilized antisense transcripts and which are capable of replicating in that host.

### Example 7

### Nucleotide Sequence Determination of Transcribed Nucleic Acid Fragments Which Inhibit Bacterial Proliferation

Using the methods described above, 8500 clones were identified in which proliferation was inhibited when transcription was induced but not under non-inducing conditioning were identified. The genomic inserts were sequenced as follows.

The polynucleotide inserts of expression vectors which, upon induction, negatively impacted E. coli growth or proliferation were subjected to nucleic acid sequence determination. The nucleotide sequences for the exogenous identified sequences were determined using plasmid DNA isolated using QIAPREP (Qiagen, Valencia, Calif.) and methods supplied by the manufacturer. The primers used for sequencing the inserts were:

5′-GTGAGCGGATAACAATGATAC-3′ (SEQ ID NO: 6) and

5′-AGGTGCCTCACTGATTAAGC-3′ (SEQ ID NO: 7)

PCR was carried out in a PE GenAmp with the following cycle times:

Step 1. 95° C. 15 min

Step 2. 94° C. 45 sec

Step 3. 54° C. 45 sec

Step 4. 72° C. 1 minute

Step 5. Return to step 2, 29 times

Step 6. 72° C. 10 minutes

Step 7. 4° C. hold

The PCR products were cleaned using Qiagen Qiaquick PCR plates according to the manufacturer's instructions. These amplified genomic DNA inserts were then subjected to automated sequencing.

### Example 8

### Comparison of the Isolated Nucleic Acid Sequences to Known Sequences

In general, antisense molecules and their complementary genes are identified as follows. First, all possible full length open reading frames (ORFs) are extracted from available genomic databases. Such databases include the GenBank nonredundant (nr) database, the unfinished genome database available from TIGR and the PathoSeq database developed by Incyte Genomics. The latter database comprises over 40 annotated bacterial genomes including complete ORF analysis. If databases are incomplete with regard to the bacterial genome of interest, it is not necessary to extract all ORFs in the genome but only to extract the ORFs within the portions of the available genomic sequences which are complementary to the clones of interest. Computer algorithms for identifying ORFs, such as GeneMark, are available and well known to those in the art. Comparison of the clone DNA to the complementary ORF(s) allows determination of whether the clone is a sense or antisense clone. Furthermore, each ORF extracted from the database can be compared to sequences in well annotated databases including the GenBank (nr) protein database, SWISSPROT and the like. A description of the gene or of a closely related gene in a closely related microorganism is often available in these databases. Similar methods are used to identify antisense clones corresponding to genes encoding non-translated RNAs.

The NCBI BLASTN 2.0.9 computer algorithm can be used to compare the nucleic acid sequences of the genomic fragments isolated from the appropriate expression vector with the extracted database sequences. The default parameters are used except that filtering is turned off. The default parameters for the BLASTN and BLASTX analyses are:

Expectation value (e)=10

Alignment view options: pairwise

Filter query sequence (DUST with BLASTN, SEG with others)=T

Cost to open a gap (zero invokes behavior)=0

Cost to extend a gap (zero invokes behavior)=0

X dropoff value for gapped alignment (in bits) (zero invokes behavior)=0

Show GI's in deflines=F

Penalty for a nucleotide mismatch (BLASTN only)=!3

Reward for a nucleotide match (BLASTN only)=1

Number of one-line descriptions (V)=500

Number of alignments to show (B)=250

Threshold for extending hits=default

Perform gapped alignment (not available with BLASTX)=T

Query Genetic code to use=1

DB Genetic code (for TBLAST[nx] only=1

Number of processors to use=1

SeqAlign file

Believe the query defline=F

Matrix=BLOSUM62

Word Size=default

Effective length of the database (use zero for the real size)=0

Number of best hits from a region to keep=100

Length of region used to judge hits=20

Effective length of the search space (use zero for the real size)=0

Query strands to search against database (for BLAST[nx] and TBLASTX), 3 is both, 1 is top, 2 is bottom=3

Produce HTML output=F

Antisense nucleic acids are identified as those fragments for which transcription from the inducible promoter would result in the expression of an RNA antisense to a complementary ORF, intergenic or intragenic sequence.

The nucleic acid sequences corresponding to genomic fragments that inhibited proliferation in E. coli were compared to known E. coli sequences in GenBank using BLAST version 2.0.6 using the following default parameters: Filtering off, cost to open a gap=5, cost to extend a gap=2, penalty for a mismatch in the BLAST portion of run=3, reward for a match in the BLAST portion of run=1, expect value (e)=10.0, word size=11. BLAST is described in Altschul, J Mol Biol. 215:403-10 (1990), the disclosure of which is incorporated herein by reference in its entirety.

Expression vectors were found to contain nucleic acids comprising nucleotide sequences in both the sense and antisense orientations. The presence of known genes, open reading frames, and ribosome binding sites was determined by the published annotation of gene content of E. coli, comparison to public databases holding genetic information and application of various programs such as The Georgia Institute of Technology's GeneMark software suite and The Institute for Genomic Research (TIGR) Glimmer software program.

Clones were designated as “antisense” if the cloned fragment was oriented to the annotated gene or predicted open reading frame such that the clone sequence was determined through BLAST sequence alignment to be complementary to the known gene or predicted open reading frame sequence. Clones were designated as “sense” if the cloned fragment sequence was determined through BLAST sequence alignment to be identical to a portion of an annotated gene or predicted open reading frame.

Over 2282 proliferation-inhibiting antisense sequences corresponding to 813 different genes were discovered. These included 623 genes which were not identified in previous analyses in which the antisense RNAs were not stabilized.

It will be appreciated that nucleic acids which inhibit proliferation in organisms other than E. coli can be isolated and identified by using methods similar to those disclosed herein and which have been described in International Publication WO 01/70955, the disclosure of which is incorporated by reference in its entirety.

### Example 9

### Identification of Genes and Corresponding Operons that are Affected by Antisense Inhibition

Once the genes involved in proliferation are identified as described above, the operons in which these genes lie can be identified by comparison with known microbial genomes. Since bacterial genes are transcribed in a polycistronic manner, the antisense inhibition of a single gene in an operon might affect the expression of all the other genes on the operon or the genes downstream from the single gene identified. Accordingly, each of the genes contained within an operon may be analyzed for their effect on proliferation.

Operons are predicted by looking for all adjacent genes in a genomic region that lie in the same orientation with no large noncoding gaps in between. First, full-length ORFs complementary to the antisense molecules are identified as described above. Adjacent ORFs are then identified and their relative orientation determined either by directly analyzing the genomic sequences surrounding the ORFs complementary to the antisense clones or by extracting adjacent ORFs from the collection obtained through whole genome ORF analysis described above followed by ORF alignment.

Operons predicted in this way can be confirmed by comparing the ORFs affecting proliferation to the arrangement of the homologous nucleic acids in the complete genome sequence of the organism from which the antisense sequence was obtained. For example, E. coli operons have been previously determined by comparing proliferation-required ORFs to the complete genomic sequence which is listed in GenBank, Accession No.U00096, the disclosure of which is incorporated herein by reference in its entirety (see, International Publication WO 00/44906, the disclosure of which is incorporated herein by reference in its entirety). Operons in Salmonella typhimurium can be determined by utilizing the extensive DNA sequences that are available for this organism through the Salmonella Genome Center (Washington University, St. Louis, Mo.) the Sanger Centre (United Kingdom) and the PathoSeq database (Incyte). Annotation of some of the DNA sequences in some of the aforementioned databases is lacking, but comparisons may be made to E. coli using tools such as BLASTX.

If insufficient genome sequence is available for the organism from which the antisense clone was obtained, the genome sequence of a closely related species can also be used. For example, operons for other gram negative bacteria such as Salmonella typhimurium and Klebsiella pneumoniae may be identified by comparison with E. coli, Haemophilus, or Pseudomonas sequences. The Pseudomonas aeruginosa web site (http://www.pseudomonas.com) can also be used to help predict operon organization in this bacterium. Predicted operons for gram positive organisms such as Staphylococcus aureus may be confirmed by comparison to the arrangement of the homologous nucleic acids in the Bacillus subtilis complete genome sequence, as reported by the genome database compiled at Institut Pasteur Subtilist Release R15.1 (Jun. 24, 1999) which can be found at http://bioweb.pasteur.fr/GenoList/SubtiList/. The Bacillus subtilis genome is the only fully sequenced and annotated genome from a gram-positive microorganism, and appears to have a high level of similarity to Staphylococcus aureus both at the level of conservation of gene sequence and genomic organization including operon structure. Combinations of public and proprietary databases may be used to analyze sequences from other organisms such as Enterococcus faecalis.

Once the full length ORFs and/or the operons containing them have been identified using the methods described above, they can be obtained from a genomic library by performing a PCR amplification using primers at each end of the desired nucleotide sequence. Those skilled in the art will appreciate that a comparison of the ORFs to homologous sequences in other cells or microorganisms will facilitate confirmation of the start and stop codons at the ends of the ORFs.

In some embodiments, the primers may contain restriction sites which facilitate the insertion of the gene or operon into a desired vector. For example, the gene may be inserted into an expression vector and used to produce the proliferation-required protein as described below. Other methods for obtaining the full length ORFs and/or operons are familiar to those of ordinary skill in the art. For example, natural restriction sites may be employed to insert the full length ORFs and/or operons into a desired vector.

### Example 10

### Identification of Individual Genes within an Operon Required for Proliferation

The following example illustrates a method for determining if a targeted gene within an operon is required for cell proliferation by replacing the targeted allele in the chromosome with an in-frame deletion of the coding region of the targeted gene.

Deletion inactivation of a chromosomal copy of a gene in E. coli can be accomplished by integrative gene replacement. The principle of this method (Xia, M., et al. 1999 Plasmid 42:144-149 and Hamilton, C. M., et al 1989. J. Bacteriol. 171: 4617-4622, the disclosures of which are incorporated herein by reference in their entireties) is to construct a mutant allele of the targeted gene, introduce that allele into the chromosome using a conditional suicide vector, and then force the removal of the native wild type allele and vector sequences. This will replace the native gene with a desired mutation(s) but leave promoters, operators, etc. intact. Essentiality of a gene is determined either by deduction from genetic analysis or by conditional expression of a wild type copy of the targeted gene (trans complementation).

This procedure can be illustrated using a nucleic acid previously identified in analyses using non-stabilized antisense RNAs which possesses sequence homology to the E. coli genes rspG and rspL. This nucleic acid also corresponds to an operon containing two additional genes fusA and tufA. The rpsL gene is the first gene in the operon. To determine which gene or genes in this operon are required for proliferation, each gene is selectively inactivated using homologous recombination. Gene rpsL is the first gene to be inactivated.

The first step is to generate a mutant rpsL allele using PCR amplification. Two sets of PCR primers are chosen to produce a copy of rpsL with a large central deletion to inactivate the gene. In order to eliminate polar effects, it is desirable to construct a mutant allele comprising an in-frame deletion of most or all of the coding region of the rpsL gene. Each set of PCR primers is chosen such that a region flanking the gene to be amplified is sufficiently long to allow recombination (typically at least 500 nucleotides on each side of the deletion). The targeted deletion or mutation will be contained within this fragment. To facilitate cloning of the PCR product, the PCR primers may also contain restriction endonuclease sites found in the cloning region of a conditional knockout vector such as pKO3 (Link, et al 1997 J. Bacteriol. 179 (20): 6228-6237). Suitable sites include NotI, SalI, BamHI and SmaI. The rpsL gene fragments are produced using standard PCR conditions including, but not limited to, those outlined in the manufacturers directions for the Hot Start Taq PCR kit (Qiagen, Inc., Valencia, Calif.). The PCR reactions will produce two fragments that can be fused together. Alternatively, crossover PCR can be used to generate a desired deletion in one step (Ho, S. N., et al 1989. Gene 77: 51-59, Horton, R. M., et al 1989. Gene 77: 61-68). The mutant allele thus produced is called a “null” allele because it cannot produce a functional gene product.

The mutant allele obtained from PCR amplification is cloned into the multiple cloning site of pKO3. Directional cloning of the rpsL null allele is not necessary. The pKO3 vector has a temperature-sensitive origin of replication derived from pSC101. Therefore, clones are propagated at the permissive temperature of 30° C. The vector also contains two selectable marker genes: one that confers resistance to chloramphenicol and another, the Bacillus subtilis sacB gene, that allows for counter-selection on sucrose containing growth medium. Clones that contain vector DNA with the null allele inserted are confirmed by restriction endonuclease analysis and DNA sequence analysis of isolated plasmid DNA. The plasmid containing the rpsL null allele insert is known as a knockout plasmid.

Once the knockout plasmid has been constructed and its nucleotide sequence verified, it is transformed into a Rec⁺ E. coli host cell. Transformation can be by any standard method such as electroporation. In some fraction of the transformed cells, plasmids will integrate into the E. coli chromosome by homologous recombination between the rpsL null allele in the plasmid and the rpsL gene in the chromosome. Transformant colonies in which such an event has occurred are readily selected by growth at the non-permissive temperature of 43° C. and in the presence of chloramphenicol. At this temperature, the plasmid will not replicate as an episome and will be lost from cells as they grow and divide. These cells are no longer resistant to chloramphenicol and will not grow when it is present. However, cells in which the knockout plasmid has integrated into the E. coli chromosome remain resistant to chloramphenicol and propagate.

Cells containing integrated knock-out plasmids are usually the result of a single crossover event that creates a tandem repeat of the mutant and native wild type alleles of rpsL separated by the vector sequences. A consequence of this is that rpsL will still be expressed in these cells. In order to determine if the gene is essential for growth, the wild type copy must be removed. This is accomplished by selecting for plasmid excision, a process in which homologous recombination between the two alleles results in looping out of the plasmid sequences. Cells that have undergone such an excision event and have lost plasmid sequences including sacB gene are selected for by addition of sucrose to the medium. The sacB gene product converts sucrose to a toxic molecule. Thus counter selection with sucrose ensures that plasmid sequences are no longer present in the cell. Loss of plasmid sequences is further confirmed by testing for sensitivity to chloramphenicol (loss of the chloramphenicol resistance gene). The latter test is important because occasionally a mutation in the sacB gene can occur resulting in a loss of sacB function with no effect on plasmid replication (Link, et. al., 1997 J. Bacteriol. 179 (20): 6228-6237). These artifact clones retain plasmid sequences and are therefore still resistant to chloramphenicol.

In the process of plasmid excision, one of the two rpsL alleles is lost from the chromosome along with the plasmid DNA. In general, it is equally likely that the null allele or the wild type allele will be lost. Therefore, if the rpsL gene is not essential, half of the clones obtained in this experiment will have the wild type allele on the chromosome and half will have the null allele. However, if the rpsL gene is essential, cells containing the null allele will not be obtained as a single copy of the null allele would be lethal.

To determine the essentiality of rpsL, a statistically significant number of the resulting clones, at least 20, are analyzed by PCR amplification of the rpsL gene. Since the null allele is missing a significant portion of the rpsL gene, its PCR product is significantly shorter than that of the wild type gene and the two are readily distinguished by gel electrophoretic analysis. The PCR products may also be subjected to sequence determination for further confirmation by methods well known to those in the art.

The above experiment is generally adequate for determining the essentiality of a gene such as rpsL. However, it may be necessary or desirable to more directly confirm the essentiality of the gene. There are several methods by which this can be accomplished. In general, these involve three steps: 1) construction of an episome containing a wild type allele, 2) isolation of clones containing a single chromosomal copy of the mutant null allele as described above but in the presence of the episomal wild type allele, and then 3) determining if the cells survive when the expression of the episomal allele is shut off. In this case, the trans copy of wild type rpsL is made by PCR cloning of the entire coding region of rpsL and inserting it in the sense orientation downstream of an inducible promoter such as the E. coli lac promoter. Transcription of this allele of rpsL will be induced in the presence of IPTG which inactivates the lac repressor. Under IPTG induction rpsL protein will be expressed as long as the recombinant gene also possesses a ribosomal binding site, also known as a “Shine-Dalgarno Sequence”. The trans copy of rpsL is cloned on a plasmid that is compatible with pSC101. Compatible vectors include p15A, pBR322, and the pUC plasmids, among others. Replication of the compatible plasmid will not be temperature-sensitive. The entire process of integrating the null allele of rpsL and subsequent plasmid excision is carried out in the presence of IPTG to ensure the expression of functional rpsL protein is maintained throughout. After the null rpsL allele is confirmed as integrated on the chromosome in place of the wild type rpsL allele, then IPTG is withdrawn and expression of functional rpsL protein shut off. If the rpsL gene is essential, cells will cease to proliferate under these conditions. However, if the rpsL gene is not essential, cells will continue to proliferate under these conditions. In this experiment, essentiality is determined by conditional expression of a wild type copy of the gene rather than inability to obtain the intended chromosomal disruption.

An advantage of this method over some other gene disruption techniques is that the targeted gene can be deleted or mutated without the introduction of large segments of foreign DNA. Therefore, polar effects on downstream genes are eliminated or minimized. There are methods described to introduce inducible promoters upstream of potential essential bacterial genes. However in such cases, polarity from multiple transcription start points can be a problem. One way of preventing this is to insert a gene disruption cassette that contains strong transcriptional terminators upstream of the integrated inducible promoter (Zhang, Y, and Cronan, J. E. 1996 J. Bacteriol. 178 (12): 3614-3620). The described techniques will all be familiar to one of ordinary skill in the art.

Following the analysis of the rpsL gene, the other genes of the operon are investigated to determine if they are required for proliferation.

Methods similar to those described above can be used in organisms other than E. coli. For example, a similar gene disruption method is available for Pseudomonas aeruginosa, except the counter selectable marker is sacB (Schweizer, H. P., Klassen, T. and Hoang, T. (1996) Mol. Biol. of Pseudomonas. ASM press, 229-237, the disclosure of which is incorporated herein by reference in its entirety). In this approach, a mutant allele of the targeted gene is constructed by way of an in-frame deletion and introduced into the chromosome using a suicide vector. This results in a tandem duplication comprising a deleted (null) allele and a wild type allele of the target gene. Cells in which the vector sequences have been deleted are isolated using a counter-selection technique. Removal of the vector sequence from the chromosomal insertion results in either restoration of the wild-type target sequence or replacement of the wild type sequence with the deletion (null) allele. E. faecalis genes can be disrupted using a suicide vector that contains an internal fragment to a gene of interest. With the appropriate selection this plasmid will homologously recombine into the chromosome (Nallapareddy, S. R., X. Qin, G. M. Weinstock, M. Hook, B. E. Murray. 2000. Infect. Immun. 68:5218-5224, the disclosure of which is incorporated herein by reference).

The method of cross-over PCR can be also be used to generate the mutant allele by amplification of nucleotide sequences flanking but not including the coding region of the gene of interest, using specifically designed primers such that overlap between the resulting two PCR amplification products allows them to hybridize. Further PCR amplification of this hybridization product using primers representing the extreme 5′ and 3′ ends can produce an amplification product containing an in-frame deletion of the coding region but retaining substantial flanking sequences. This cross-over PCR product can then be introduced into the host genome by using the appropriate suicide vector.

As described above, the resultant cell population can then be evaluated to determine whether the target sequence is required for proliferation by PCR amplification of the affected target sequence. If the targeted gene is not required for proliferation, then PCR analysis will show that roughly equal numbers of colonies have retained either the wild-type or the mutant allele. If the targeted gene is required for proliferation, then only wild-type alleles will be recovered in the PCR analysis.

The above methods have the advantage that insertion of an in-frame deletion mutation is far less likely to cause downstream polar effects on genes in the same operon as the targeted gene. However, it will be appreciated that other methods for disrupting genes in microorganisms which are familiar to those of ordinary skill in the art may also be used.

Each gene in the operon may be disrupted using the methodology above to determine whether it is required for proliferation.

### Example 11

### Screening Chemical Libraries in Cells Sensitized with Stabilized Antisense RNA

Current cell-based assays used to identify or to characterize compounds for drug discovery and development frequently depend on detecting the ability of a test compound to inhibit the activity of a target molecule located within a cell or located on the surface of a cell. Most often such target molecules are proteins such as enzymes, receptors and the like. However, target molecules may also include other molecules such as DNAs, lipids, carbohydrates and RNAs including messenger RNAs, ribosomal RNAs, tRNAs and the like. A number of highly sensitive cell-based assay methods are available to those of skill in the art to detect binding and interaction of test compounds with specific target molecules. However, these methods are generally not highly effective when the test compound binds to or otherwise interacts with its target molecule with moderate or low affinity. In addition, the target molecule may not be readily accessible to a test compound in solution, such as when the target molecule is located inside the cell or within a cellular compartment such as the periplasm of a bacterial cell. Thus, current cell-based assay methods are limited in that they are not effective in identifying or characterizing compounds that interact with their targets with moderate to low affinity or compounds that interact with targets that are not readily accessible.

Cell-based assay methods of the present invention have substantial advantages over current cell-based assays practiced in the art. These advantages derive from the use of sensitized cells in which the level or activity of a proliferation-required gene product (the target molecule) has been specifically reduced to the point where the presence or absence of its function becomes a rate-determining step for cellular proliferation. Bacterial, fungal, plant, or animal cells can all be used with the present method. Such sensitized cells become much more sensitive to compounds that are active against the affected target molecule. Thus, cell-based assays of the present invention are capable of detecting compounds exhibiting low or moderate potency against the target molecule of interest because such compounds are substantially more potent on sensitized cells than on non-sensitized cells. The affect may be such that a test compound may be two to several times more potent, at least 10 times more potent or even at least 100 times more potent when tested on the sensitized cells as compared to the non-sensitized cells.

Due in part to the increased appearance of antibiotic resistance in pathogenic microorganisms and to the significant side-effects associated with some currently used antibiotics, novel antibiotics acting at new targets are highly sought after in the art. Yet, another limitation in the current art related to cell-based assays is the problem of identifying hits against the same kinds of target molecules in the same limited set of biological pathways over and over again. This may occur when compounds acting at such new targets are discarded, ignored or fail to be detected because compounds acting at the “old” targets are encountered more frequently and are more potent than compounds acting at the new targets. As a result, the majority of antibiotics in use currently interact with a relatively small number of target molecules within an even more limited set of biological pathways.

The use of sensitized cells provides a solution to the above problem in two ways. First, desired compounds acting at a target of interest, whether a new target or a previously known but poorly exploited target, can now be detected above the “noise” of compounds acting at the “old” targets due to the specific and substantial increase in potency of such desired compounds when tested on the sensitized cells. Second, the methods used to sensitize cells to compounds acting at a target of interest may also sensitize these cells to compounds acting at other target molecules within the same biological pathway. For example, expression of a stabilized antisense molecule to a gene encoding a ribosomal protein is expected to sensitize the cell to compounds acting at that ribosomal protein and may also sensitize the cells to compounds acting at any of the ribosomal components (proteins or rRNA) or even to compounds acting at any target which is part of the protein synthesis pathway. Thus an important advantage of this method is the ability to reveal new targets and pathways that were previously not readily accessible to drug discovery methods.

Sensitized cells to be used in the screening of chemical compounds are prepared by reducing the activity or level of a target molecule. The target molecule may be a gene product, such as an RNA or polypeptide produced from proliferation-required nucleic acids. Alternatively, the target may be a gene product such as an RNA or polypeptide which is produced from a nucleotide sequence within the same operon as a proliferation-required nucleic acid. In addition, the target may be an RNA or polypeptide in the same biological pathway as a proliferation-required nucleic acid. Such biological pathways include, but are not limited to, enzymatic, biochemical and metabolic pathways as well as pathways involved in the production of cellular structures such the cell wall.

Current methods employed in the arts of medicinal and combinatorial chemistries are able to make use of structure-activity relationship information derived from testing compounds in various biological assays including direct binding assays and cell-based assays. Occasionally compounds are directly identified in such assays that are sufficiently potent to be developed as drugs. More often, initial hit compounds exhibit moderate or low potency. Once a hit compound is identified with low or moderate potency, directed libraries of compounds are synthesized and tested in order to identify more potent leads. Generally these directed libraries are combinatorial chemical libraries consisting of compounds with structures related to the hit compound but containing systematic variations including additions, subtractions and substitutions of various structural features. When tested for activity against the target molecule, structural features are identified that either alone or in combination with other features enhance or reduce activity. This information is used to design subsequent directed libraries containing compounds with enhanced activity against the target molecule. After one or several iterations of this process, compounds with substantially increased activity against the target molecule are identified and may be further developed as drugs. This process is facilitated by use of the sensitized cells of the present invention since compounds acting at the selected targets exhibit increased potency in such cell-based assays, thus; more compounds can now be characterized providing more useful information than would be obtained otherwise.

Thus, it is now possible using cell-based assays of the present invention to identify or characterize compounds that previously would not have been readily identified or characterized including compounds that act at targets that previously were not readily exploited using cell-based assays. The process of evolving potent drug leads from initial hit compounds is also substantially improved by the cell-based assays of the present invention because, for the same number of test compounds, more structure-function relationship information is likely to be revealed.

The method of sensitizing a cell entails selecting a suitable gene or operon. A suitable gene or operon is one whose expression is required for the proliferation of the cell to be sensitized. The next step is to introduce into the cells to be sensitized, a stabilized antisense RNA capable of hybridizing to the suitable gene or operon or to the RNA encoded by the suitable gene or operon. Introduction of the stabilized antisense RNA can be in the form of an expression vector in which the stabilized antisense RNA is produced under the control of an inducible promoter. The amount of stabilized antisense RNA produced is limited by varying the inducer concentration to which the cell is exposed and thereby varying the activity of the promoter driving transcription of the stabilized antisense RNA. Thus, cells are sensitized by exposing them to an inducer concentration that results in a sub-lethal level of stabilized antisense RNA expression.

Compared to cells that are sensitized by nonstabilized RNA, cells that contain stabilized antisense RNA suffer a further reduction in the functional expression of the proliferation-required gene target and thus become more sensitive to marginally active antibiotics. Although many antisense RNAs may be produced using high-levels or inducer, rapid RNA degradation can lead to a low effective concentration of antisense molecules in the cell. Expression of stabilized antisense RNA molecules in a host having reduced ability to degrade RNA increases the lifetime and thus the effective concentration of these molecules. Cells that contain stable antisense molecules show a reduced level of proliferation-required gene expression when compared to similarly induced cells that are sensitized by nonstabilized antisense molecules. The corresponding decrease in target protein further increases the sensitivity of the cell to marginally active antibiotics. Additionally, sensitizing cells with stable antisense RNA molecules reduces the expression of proliferation-required genes that are not normally inhibited by short-lived antisense molecules thereby enhancing the detection of these potentially novel antibiotic targets.

In one embodiment of the current invention, the transcript stabilizing vectors described herein may be used for antisense RNA expression. In another embodiment, the antisense RNA can be stabilized by expression in mutant host strains having a reduced ability to degrade RNA. In yet another embodiment, the RNA stabilization methods disclosed herein can be combined for use in the above cell-based assays.

In one embodiment of the cell-based assays, stabilized antisense nucleic acids which are complementary to least a portion of an E. coli gene encoding E. coli a proliferation-required protein are used. Expression vectors producing stabilized antisense RNA against identified genes required for proliferation are used to limit the concentration of a proliferation-required protein without severely inhibiting growth. To achieve that goal, a growth inhibition dose curve of inducer is calculated by plotting various doses of inducer against the corresponding growth inhibition caused by the antisense expression. From this curve, various percentages of antisense induced growth inhibition, from 1 to 100% can be determined. If the promoter contained in the expression vector contains a lac operator the transcription is regulated by lac repressor and expression from the promoter is inducible with IPTG. For example, the highest concentration of the inducer IPTG that does not reduce the growth rate (0% growth inhibition) can be predicted from the curve. Cellular proliferation can be monitored by growth medium turbidity via OD measurements. In another example, the concentration of inducer that reduces growth by 25% can be predicted from the curve. In still another example, a concentration of inducer that reduces growth by 50% can be calculated. Additional parameters such as colony forming units (cfu) can be used to measure cellular viability.

Cells to be assayed are exposed to the above-determined concentrations of inducer. The presence of the inducer at this sub-lethal concentration reduces the amount of the proliferation required gene product to the lowest amount in the cell that will support growth. Cells grown in the presence of this concentration of inducer are therefore specifically more sensitive to inhibitors of the proliferation-required protein or RNA of interest or to inhibitors of proteins or RNAs in the same biological pathway as the proliferation-required protein or RNA of interest but not to inhibitors of unrelated proteins or RNAs.

Cells pretreated with sub-inhibitory concentrations of inducer and thus containing a reduced amount of proliferation-required target gene product are then used to screen for compounds that reduce cell growth. The sub-lethal concentration of inducer may be any concentration consistent with the intended use of the assay to identify candidate compounds to which the cells are more sensitive. For example, the sub-lethal concentration of the inducer may be such that growth inhibition is at least about 5%, at least about 8%, at least about 10%, at least about 20%, at least about 30%, at least about 40%, at least about 50%, at least about 60% at least about 75%, or more. Cells which are pre-sensitized using the preceding method are more sensitive to inhibitors of the target protein because these cells contain less target protein to inhibit than wild-type cells.

It will also be appreciated that the above cell-based assays may be performed using antisense nucleic acids complementary to any of the proliferation-required nucleic acids from organisms other than E. coli, or portions thereof, antisense nucleic acids complementary to homologous coding nucleic acids or portions thereof, or homologous antisense nucleic acids. In this way, the level or activity of a target, such as any of the proliferation-required polypeptides from organisms other than E. coli, or homologous polypeptides may be reduced.

An artisan of ordinary skill will appreciate that the methods for stabilizing RNA disclosed herein can also be used in organisms other than E. coli. One of ordinary skill in the art will recognize that vectors for the stabilized expression of RNA in organisms other than E. coli, including shuttle vectors, can be constructed using the techniques disclosed herein. A skilled artisan will also recognize that host organisms other than E. coli that have a reduced ability to degrade RNA can be used for the stabilized expression of RNA. Furthermore, one skilled in the art will appreciate that both stabilization methods can be used together and in conjunction with the above cell-based assays.

In particular, the above cell-based assays employing methods of stabilizing RNA expression can be used with organisms including, but not limited to, Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species. In one embodiment of the present invention, these organisms include gram-negative bacteria.

The sensitivity of cell based assays can also be enhanced by reducing the level or activity of a proliferation-required gene product by using a temperature sensitive mutation in the proliferation-required sequence and an antisense nucleic acid against the proliferation-required sequence. Growing the cells at an intermediate temperature between the permissive and restrictive temperatures of the temperature sensitive mutant where the mutation is in a proliferation-required gene produces cells with reduced activity of the proliferation-required gene product. The antisense RNA directed against the proliferation-required sequence further reduces the activity of the proliferation required gene product. Drugs that may not have been found using either the temperature sensitive mutation or the stabilized antisense nucleic acid alone may be identified by determining whether cells in which expression of the stabilized antisense nucleic acid has been induced and which are grown at a temperature between the permissive temperature and the restrictive temperature are substantially more sensitive to a test compound than cells in which expression of the stabilized antisense nucleic acid has not been induced and which are grown at a permissive temperature. Also drugs found previously from either the stabilized antisense nucleic acid alone or the temperature sensitive mutation alone may have a different sensitivity profile when used in cells combining the two approaches, and that sensitivity profile may indicate a more specific action of the drug in inhibiting one or more activities of the gene product.

Temperature sensitive mutations may be located at different sites within the gene and correspond to different domains of the protein. For example, the dnaB gene of Escherichia coli encodes the replication fork DNA helicase. DnaB has several domains, including domains for oligomerization, ATP hydrolysis, DNA binding, interaction with primase, interaction with DnaC, and interaction with DnaA [(Biswas, E. E. and Biswas, S. B. 1999. Mechanism and DnaB helicase of Escherichia coli: structural domains involved in ATP hydrolysis, DNA binding, and oligomerization. Biochem. 38:10919-10928; Hiasa, H. and Marians, K. J. 1999. Initiation of bidirectional replication at the chromosomal origin is directed by the interaction between helicase and primase. J. Biol. Chem. 274:27244-27248; San Martin, C., Radermacher, M., Wolpensinger, B., Engel, A., Miles, C. S., Dixon, N. E., and Carazo, J. M. 1998. Three-dimensional reconstructions from cryoelectron microscopy images reveal an intimate complex between helicase DnaB and its loading partner DnaC. Structure 6:501-9; Sutton, M. D., Carr, K. M., Vicente, M., and Kaguni, J. M. 1998. Escherichia coli DnaA protein. The N-terminal domain and loading of DnaB helicase at the E. coli chromosomal. J. Biol. Chem. 273:34255-62.), the disclosures of which are incorporated herein by reference in their entireties]. Temperature sensitive mutations in different domains of DnaB confer different phenotypes at the restrictive temperature, which include either an abrupt stop or slow stop in DNA replication with or without DNA breakdown (Wechsler, J. A. and Gross, J. D. 1971. Escherichia coli mutants temperature-sensitive for DNA synthesis. Mol. Gen. Genetics 113:273-284, the disclosure of which is incorporated herein by reference in its entirety) and termination of growth or cell death. Combining the use of temperature sensitive mutations in the dnaB gene that cause cell death at the restrictive temperature with a stabilized antisense to the dnaB gene could lead to the discovery of very specific and effective inhibitors of one or a subset of activities exhibited by DnaB.

It will be appreciated that cell-based assays such as those described above can be used alone or in combination. For example, a temperature sensitive mutation to a proliferation-required gene in a host having reduced ability to degrade RNA can be isolated. An RNA stabilizing expression vector, such as pEPEC3, containing a nucleic acid complementary to at least a portion of the mutant proliferation-required gene is transformed into the host. The host is then grown under semi-restrictive conditions in the presence of appropriate concentrations of inducer so as to express a stabilized antisense molecule having a stem-loop structure at each end.

When screening for antimicrobial agents against a gene product required for proliferation, growth inhibition of cells containing a limiting amount of that proliferation-required gene product can be assayed. Growth inhibition can be measured by directly comparing the amount of growth, measured by the optical density of the growth medium, between an experimental sample and a control sample. Alternative methods for assaying cell proliferation include measuring green fluorescent protein (GFP) reporter construct emissions, various enzymatic activity assays, and other methods well known in the art.

It will be appreciated that the above method may be performed in solid phase, liquid phase or a combination of the two. For example, cells grown on nutrient agar containing the inducer of the antisense construct may be exposed to compounds spotted onto the agar surface. A compound's effect may be judged from the diameter of the resulting killing zone, the area around the compound application point in which cells do not grow. Multiple compounds may be transferred to agar plates and simultaneously tested using automated and semi-automated equipment including but not restricted to multi-channel pipettes (for example the Beckman Multimek) and multi-channel spotters (for example the Genomic Solutions Flexys). In this way multiple plates and thousands to millions of compounds may be tested per day.

The compounds may also be tested entirely in liquid phase using microtiter plates as described below. Liquid phase screening may be performed in microtiter plates containing 96, 384, 1536 or more wells per microtiter plate to screen multiple plates and thousands to millions of compounds per day. Automated and semi-automated equipment may be used for addition of reagents (for example cells and compounds) and determination of cell density. In some embodiments the host organism is grown with vigorous shaking to ensure homogenous distribution throughout the growth medium and thus, accurate measurement of proliferation ability.

### Example 12

### Cell-Based Assay Using Antisense Complementary to Genes Encoding Ribosomal Proteins

The effectiveness of the above cell-based assays in which the level or activity of a gene product required for proliferation was reduced was validated using constructs transcribing a non-stabilized antisense RNA to the proliferation required E. coli genes rplL, rplJ and rplW encoding ribosomal proteins L7/L12, L10 and L23 respectively. These proteins are essential components of the protein synthesis apparatus of the cell and as such are required for proliferation. Constructs transcribing antisense RNA to several other genes (elaD, visC, yohH, and atpE/B), the products of which are not involved in protein synthesis were used for comparison.

First, pLex5BA (Krause et al., J. Mol. Biol. 274: 365 (1997), the disclosure of which is incorporated herein by reference in its entirety) vectors containing antisense constructs to either rplW or to elaD were introduced into separate E. coli cell populations. Vector introduction is a technique well known to those of ordinary skill in the art. The vectors of this example contain IPTG inducible promoters that drive the transcription of the antisense RNA in the presence of the inducer. However, those skilled in the art will appreciate that other inducible promoters may also be used. Antisense clones to genes encoding different ribosomal proteins or to genes encoding proteins that are not involved in protein synthesis were utilized to test the effect of antisense transcription on cell sensitivity to the antibiotics known to bind to ribosomal proteins and inhibit protein synthesis. Antisense nucleic acids comprising a nucleotide sequence complementary to the elaD, atpB&atpE, visC and yohH genes are referred to as AS-elaD, AS-atpB/E, AS-visC, AS-yohH respectively. These genes are not known to be involved in protein synthesis. Antisense nucleic acids to the rplL, rplL&rplJ and rplW genes are referred to as AS-rplL, AS-rplL/J, and AS-rplW respectively. These genes encode ribosomal proteins L7/L12 (rplL) L10 (rplJ) and L23 (rplW). Vectors containing these antisense nucleic acids were introduced into separate E. coli cell populations.

The cell populations containing vectors producing AS-elaD or AS-rplW were exposed to a range of IPTG concentrations in liquid medium to obtain the growth inhibitory dose curve for each clone (FIG. 5). First, seed cultures were grown to a particular turbidity measured by the optical density (OD) of the growth solution. The OD of the solution is directly related to the number of bacterial cells contained therein. Subsequently, sixteen 200 μl liquid medium cultures were grown in a 96 well microtiter plate at 37° C. with a range of IPTG concentrations in duplicate two-fold serial dilutions from 1600 uM to 12.5 μM (final concentration). Additionally, control cells were grown in duplicate without IPTG. These cultures were started from an inoculum of equal amounts of cells derived from the same initial seed culture of a clone of interest. The cells were grown for up to 15 hours and the extent of growth was determined by measuring the optical density of the cultures at 600 nm. When the control culture reached mid-log phase the percent growth (relative to the control culture) for each of the IPTG containing cultures was plotted against the log concentrations of IPTG to produce a growth inhibitory dose response curve for the IPTG. The concentration of IPTG that inhibits cell growth to 50% (IC₅₀) as compared to the 0 mM IPTG control (0% growth inhibition) was then calculated from the curve. Under these conditions, an amount of antisense RNA was produced that reduced the expression levels of rplW or elaD to a degree such that growth of cells containing their respective antisense vectors was inhibited by 50%.

Alternative methods of measuring growth are also contemplated. Examples of these methods include measurements of proteins, the expression of which is engineered into the cells being tested and can readily be measured. Examples of such proteins include green fluorescent protein (GFP), luciferase, and various enzymes.

Cells were pretreated with the selected concentration of IPTG and then used to test the sensitivity of cell populations to tetracycline, erythromycin and other known protein synthesis inhibitors. FIG. 5 is an IPTG dose response curve in E. coli transformed with an IPTG-inducible plasmid containing either an antisense clone to the E. coli rplW gene (AS-rplW) which encodes ribosomal protein L23 which is required for protein synthesis and essential for cell proliferation, or an antisense clone to the elaD (AS-elaD) gene which is not known to be involved in protein synthesis.

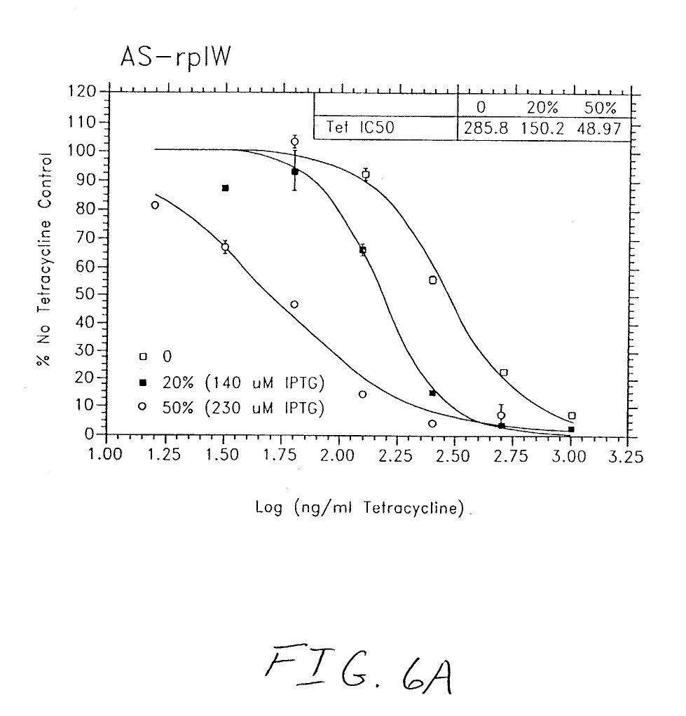

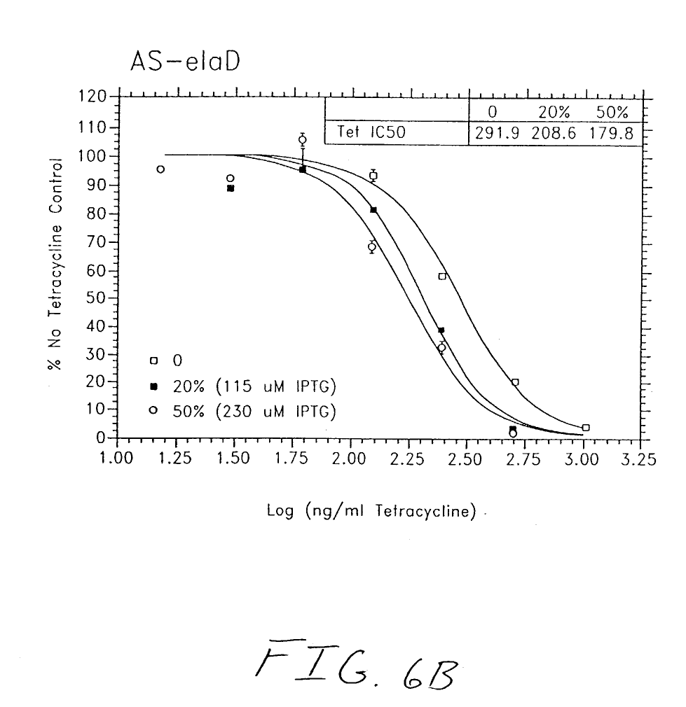

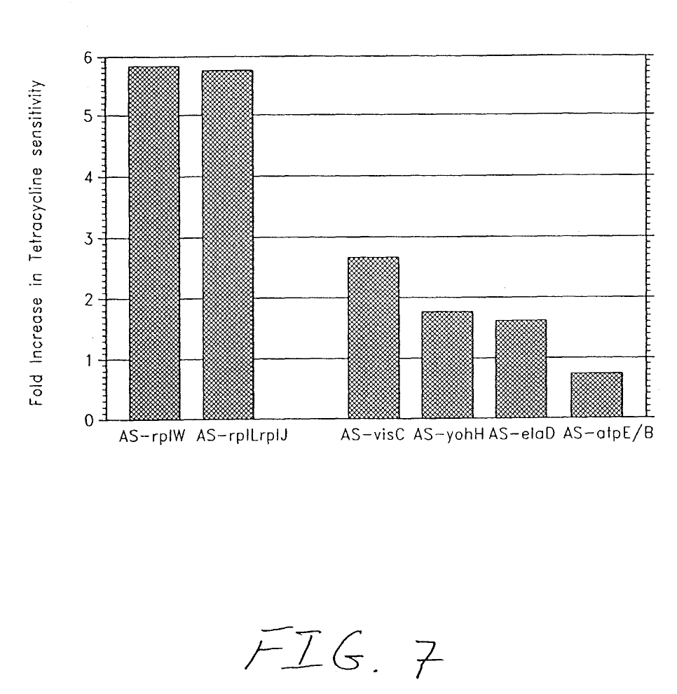

An example of a tetracycline dose response curve is shown in FIGS. 6A and 6B for the rplW and elaD genes, respectively. Cells were grown to log phase and then diluted into medium alone or medium containing IPTG at concentrations which give 20% and 50% growth inhibition as determined by IPTG dose response curves. After 2.5 hours, the cells were diluted to a final OD₆₀₀ of 0.002 into 96 well plates containing (1) +/−IPTG at the same concentrations used for the 2.5 hour pre-incubation; and (2) serial two-fold dilutions of tetracycline such that the final concentrations of tetracycline range from 1 μg/ml to 15.6 ng/ml and 0 μg/ml. The 96 well plates were incubated at 37° C. and the OD₆₀₀ was read by a plate reader every 5 minutes for up to 15 hours. For each IPTG concentration and the no IPTG control, tetracycline dose response curves were determined when the control (absence of tetracycline) reached 0.1 OD₆₀₀.

To compare tetracycline sensitivity with and without IPTG, tetracycline IC₅₀ₛ were determined from the dose response curves (FIGS. 7A and 7B). Cells transcribing antisense nucleic acids AS-rplL or AS-rplW to genes encoding ribosomal proteins L7/L12 and L23 respectively showed increased sensitivity to tetracycline (FIG. 6A) as compared to cells with reduced levels of the elaD gene product (AS-elaD) (FIG. 6B). FIG. 3 shows a summary bar chart in which the ratios of tetracycline IC₅₀ₛ determined in the presence of IPTG which gives 50% growth inhibition versus tetracycline IC₅₀ₛ determined without IPTG (fold increase in tetracycline sensitivity) were plotted. Cells with reduced levels of either L7/L12 (encoded by genes rplL, rplJ) or L23 (encoded by the rplW gene) showed increased sensitivity to tetracycline (FIG. 7). Cells expressing antisense to genes not known to be involved in protein synthesis (AS-atpB/E, AS-visC, AS-elaD, AS-yohH) did not show the same increased sensitivity to tetracycline, validating the specificity of this assay (FIG. 7).

In addition to the above, it has been observed in initial experiments that clones transcribing antisense RNA to genes involved in protein synthesis (including genes encoding ribosomal proteins L7/L12 & L10, L7/L12 alone, L22, and L18, as well as genes encoding rRNA and Elongation Factor G) have increased sensitivity to the macrolide, erythromycin, whereas clones transcribing antisense to the non-protein synthesis genes elaD, atpB/E and visC do not. Furthermore, the clone transcribing antisense to rplL and rplJ (AS-rplL/J) does not show increased sensitivity to nalidixic acid and ofloxacin, antibiotics which do not inhibit protein synthesis.

The results with the ribosomal protein genes rplL, rplJ, and rplW as well as the initial results using various other antisense clones and antibiotics show that limiting the concentration of an antibiotic target makes cells more sensitive to the antimicrobial agents that specifically interact with that protein. The results also show that these cells are sensitized to antimicrobial agents that inhibit the overall function in which the protein target is involved but are not sensitized to antimicrobial agents that inhibit other functions.

It will be appreciated that the above cell-based assays may be performed in conjunction with the methods for stabilizing RNA disclosed herein. In one embodiment of the current invention, the vectors for transcribing stabilized antisense RNAs described herein may be used. In another embodiment, the antisense RNA can be stabilized by expression in mutant host strains having a reduced ability to degrade RNA. In yet another embodiment, the RNA stabilization methods disclosed herein can be combined for use in the above cell-based assays.

It will also be appreciated that the above cell-based assays may be performed using stabilized antisense nucleic acids complementary to any of the proliferation-required nucleic acids identified as described herein, or portions thereof, antisense nucleic acids complementary to homologous coding nucleic acids or portions thereof, or homologous antisense nucleic acids. In this way, the level or activity of a target, such as a proliferation-required polypeptides from a heterologous organism may be reduced.

A skilled artisan will appreciate that the methods for stabilizing RNA disclosed herein can also be used in organisms other than E. coli. One skilled in the art will recognize that vectors for the transcription of stabilized antisense RNA in organisms other than E. coli, including shuttle vectors, can be constructed using the techniques disclosed herein. In one embodiment, the vector described in U.S. Patent Application Serial No. 60/259,434, the disclosure of which is incorporated by reference in its entirety, may be modified to transcribe stabilized antisense RNAs. A skilled artisan will also recognize that host organisms other than E. coli that have a reduced ability to degrade RNA can be used for the stabilized expression of RNA. Furthermore, one skilled in the art will appreciate that both stabilization methods can be used together and in conjunction with the above cell-based assays.

In particular, the above cell-based assays employing methods of stabilizing RNA expression can be used with organisms including, but not limited to, Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species. In one embodiment of the current invention, these organisms include gram-negative bacteria. In some embodiments the host organism is grown with vigorous shaking to ensure homogenous distribution throughout the growth medium and thus, accurate measurement of proliferation ability.

The cell-based assay described above may also be used to identify the biological pathway in which a proliferation-required nucleic acid or its gene product lies. In such methods, cells transcribing a sub-lethal level of a stabilized antisense RNA complementary to a target proliferation-required nucleic acid and control cells in which transcription of the stabilized antisense RNA has not been induced are contacted with a panel of antibiotics known to act in various pathways. If the antibiotic acts in the pathway in which the target proliferation-required nucleic acid or its gene product lies, cells in which transcription of the stabilized antisense RNA has been induced will be more sensitive to the antibiotic than cells in which expression of the stabilized antisense RNA has not been induced.

As a control, the results of the assay may be confirmed by contacting a panel of cells transcribing stabilized antisense nucleic acids complementary to at least a portion of many different proliferation-required genes including the target proliferation-required gene. If the antibiotic is acting specifically, heightened sensitivity to the antibiotic will be observed only in the cells transcribing stabilized antisense RNA complementary to a target proliferation-required gene (or cells expressing stabilized antisense RNA complementary to other proliferation-required genes in the same pathway as the target proliferation-required gene) but will not be observed generally in all cells expressing stabilized antisense RNA complementary to proliferation-required genes.

In some embodiments, the antisense RNA can be stabilized by expression in mutant host strains having a reduced ability to degrade RNA. In yet another embodiment, the RNA stabilization methods disclosed herein (i.e. stabilization by transcribing antisense RNAs flanked on each end by at least one stem-loop and stabilization by transcription of antisense RNA in hosts having reduced abillity to degrade RNA) can be combined for use in the above cell-based assays.

It will also be appreciated that the above cell-based assays may be performed using stabilized antisense nucleic acids complementary to any of the proliferation-required nucleic acids identified as described herein, or portions thereof, antisense nucleic acids complementary to homologous coding nucleic acids or portions thereof, or homologous antisense nucleic acids. In this way, the level or activity of a target, such as a proliferation-required polypeptide from a heterologous organism may be reduced.

A skilled artisan will appreciate that the methods for stabilizing RNA disclosed herein can also be used in organisms other than E. coli. One skilled in the art will recognize that vectors for the transcription of stabilized antisense RNA in organisms other than E. coli, including shuttle vectors, can be constructed using the techniques disclosed herein. In one embodiment, the vector described in U.S. Patent Application Serial No. 60/259,434, the disclosure of which is incorporated by reference in its entirety, may be modified to transcribe stabilized antisense RNAs. A skilled artisan will also recognize that host organisms other than E. coli that have a reduced ability to degrade RNA can be used for the stabilized expression of RNA. Furthermore, one skilled in the art will appreciate that both stabilization methods can be used together and in conjunction with the above cell-based assays.

In particular, the above cell-based assays employing methods of stabilizing RNA expression can be used with organisms including, but not limited to, Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species. In one embodiment of the current invention, these organisms include gram-negative bacteria. In some embodiments the host organism is grown with vigorous shaking to ensure homogenous distribution throughout the growth medium and thus, accurate measurement of proliferation ability.

Similarly, the above method may be used to determine the pathway on which a test compound, such as a test antibiotic acts. A panel of cells, each of which transcribes a stabilized antisense RNA complementary to at least a portion of a proliferation-required nucleic acid in a known pathway, is contacted with a compound for which it is desired to determine the pathway on which it acts. The sensitivity of the panel of cells to the test compound is determined in cells in which transcription of the stabilized antisense RNA has been induced and in control cells in which expression of the stabilized antisense RNA has not been induced. If the test compound acts on the pathway on which a stabilized antisense nucleic acid acts, cells in which expression of the stabilized antisense nucleic acid has been induced will be more sensitive to the compound than cells in which expression of the stabilized antisense nucleic acids has not been induced. In addition, control cells in which transcription of stabilized antisense nucleic acids complementary to proliferation-required genes in other pathways has been induced will not exhibit heightened sensitivity to the compound. In this way, the pathway on which the test compound acts may be determined.

In some embodiments, the antisense RNA can be stabilized by expression in mutant host strains having a reduced ability to degrade RNA. In yet another embodiment, the RNA stabilization methods disclosed herein (i.e. stabilization by transcribing antisense RNAs flanked on each end by at least one stem-loop and stabilization by transcription of antisense RNA in hosts having reduced abillity to degrade RNA) can be combined for use in the above cell-based assays.

It will also be appreciated that the above cell-based assays may be performed using stabilized antisense nucleic acids complementary to any of the proliferation-required nucleic acids identified as described herein, or portions thereof, antisense nucleic acids complementary to homologous coding nucleic acids or portions thereof, or homologous antisense nucleic acids. In this way, the level or activity of a target, such as a proliferation-required polypeptide from a heterologous organism may be reduced.

A skilled artisan will appreciate that the methods for stabilizing RNA disclosed herein can also be used in organisms other than E. coli. One skilled in the art will recognize that vectors for the transcription of stabilized antisense RNA in organisms other than E. coli, including shuttle vectors, can be constructed using the techniques disclosed. herein. In one embodiment, the vector described in U.S. patent application Ser. No. 60/259,434, the disclosure of which is incorporated by reference in its entirety, may be modified to transcribe stabilized antisense RNAs. A skilled artisan will also recognize that host organisms other than E. coli that have a reduced ability to degrade RNA can be used for the stabilized expression of RNA. Furthermore, one skilled in the art will appreciate that both stabilization methods can be used together and in conjunction with the above cell-based assays.

In particular, the above cell-based assays employing methods of stabilizing RNA expression can be used with organisms including, but not limited to, Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species. In one embodiment of the current invention, these organisms include gram-negative bacteria. In some embodiments the host organism is grown with vigorous shaking to ensure homogenous distribution throughout the growth medium and thus, accurate measurement of proliferation ability.

The Example below provides one method for performing such assays.

### Example 13

### Identification of the Pathway in Which a Proliferation-Required Gene Lies or the Pathway on Which an Antibiotic Acts

A. Preparation of Bacterial Stocks for Assay

To provide a consistent source of cells to screen, frozen stocks of host bacteria containing the desired antisense construct are prepared using standard microbiological techniques. For example, a single clone of the microorganism can be isolated by streaking out a sample of the original stock onto an agar plate containing nutrients for cell growth and an antibiotic for which the antisense construct contains a selectable marker which confers resistance. After overnight growth an isolated colony is picked from the plate with a sterile needle and transferred to an appropriate liquid growth medium containing the antibiotic required for maintenance of the plasmid. The cells are incubated at 30° C. to 37° C. with vigorous shaking for 4 to 6 hours to yield a culture in exponential growth. Sterile glycerol is added to 15% (volume to volume) and 100 μL to 500 μL aliquots are distributed into sterile cryotubes, snap frozen in liquid nitrogen, and stored at −80° C. for future assays.

B. Growth of Bacteria for Use in the Assay

A day prior to an assay, a stock vial is removed from the freezer, rapidly thawed (37° C. water bath) and a loop of culture is streaked out on an agar plate containing nutrients for cell growth and an antibiotic to which the selectable marker of the antisense construct confers resistance. After overnight growth at 37° C., ten randomly chosen, isolated colonies are transferred from the plate (sterile inoculum loop) to a sterile tube containing 5 mL of LB medium containing the antibiotic to which the stabilized antisense vector confers resistance. After vigorous mixing to form a homogeneous cell suspension, the optical density of the suspension is measured at 600 nm (OD₆₀₀) and if necessary an aliquot of the suspension is diluted into a second tube of 5 mL, sterile, LB medium plus antibiotic to achieve an OD₆₀₀<0.02 absorbance units. The culture is then incubated at 37° C. for 1-2 hrs with shaking until the OD₆₀₀ reaches OD 0.2-0.3. At this point the cells are ready to be used in the assay.

C. Selection of Media to be Used in Assay

Two-fold dilution series of the inducer are generated in culture media containing the appropriate antibiotic for maintenance of the stabilized antisense construct. Several media are tested side by side and three to four wells are used to evaluate the effects of the inducer at each concentration in each media. For example, M9 minimal media, LB broth, TBD broth and Muller-Hinton media may be tested with the inducer IPTG at the following concentrations, 50 μM, 100 μM, 200 μM, 400 μM, 600 μM, 800 μM and 1000 μM. Equal volumes of test media-inducer and cells are added to the wells of a 384 well microtiter plate and mixed. The cells are prepared as described above and diluted 1:100 in the appropriate media containing the test antibiotic immediately prior to addition to the microtiter plate wells. For a control, cells are also added to several wells of each media that do not contain inducer, for example 0 mM IPTG. Cell growth is monitored continuously by incubation at 37° C. in a microtiter plate reader monitoring the OD₆₀₀ of the wells over an 18-hour period. The percent inhibition of growth produced by each concentration of inducer is calculated by comparing the rates of logarithmic growth against that exhibited by cells growing in medium without inducer. The medium yielding greatest sensitivity to inducer is selected for use in the assays described below.

D. Measurement of Test Antibiotic Sensitivity in the Absence of Antisense Construct Induction

Two-fold dilution series of antibiotics of known mechanism of action are generated in the culture medium selected for further assay development that has been supplemented with the antibiotic used to maintain the construct. A panel of test antibiotics known to act on different pathways is tested side by side with three to four wells being used to evaluate the effect of a test antibiotic on cell growth at each concentration. Equal volumes of test antibiotic and cells are added to the wells of a 384 well microtiter plate and mixed. Cells are prepared as described above using the medium selected for assay development supplemented with the antibiotic required to maintain the antisense construct and are diluted 1:100 in identical medium immediately prior to addition to the microtiter plate wells. For a control, cells are also added to several wells that lack antibiotic, but contain the solvent used to dissolve the antibiotics. Cell growth is monitored continuously by incubation at 37° C. in a microtiter plate reader monitoring the OD₆₀₀ of the wells over an 18-hour period. The percent inhibition of growth produced by each concentration of antibiotic is calculated by comparing the rates of logarithmic growth against that exhibited by cells growing in medium without antibiotic. A plot of percent inhibition against log[antibiotic concentration] allows extrapolation of an IC₅₀ value for each antibiotic.

E. Measurement of Test Antibiotic Sensitivity in the Presence of Antisense Construct Inducer

The culture medium selected for use in the assay is supplemented with inducer at concentrations shown to inhibit cell growth by 50% and 80% as described above, as well as the antibiotic used to maintain the construct. Two-fold dilution series of the panel of test antibiotics used above are generated in each of these media. Several antibiotics are tested side by side in each medium with three to four wells being used to evaluate the effects of an antibiotic on cell growth at each concentration. Equal volumes of test antibiotic and cells are added to the wells of a 384 well microtiter plate and mixed. Cells are prepared as described above using the medium selected for use in the assay supplemented with the antibiotic required to maintain the antisense construct. The cells are diluted 1:100 into two 50 mL aliquots of identical medium containing concentrations of inducer that have been shown to inhibit cell growth by 50% and 80% respectively and incubated at 37° C. with shaking for 2.5 hours. Immediately prior to addition to the microtiter plate wells, the cultures are adjusted to an appropriate OD₆₀₀ (typically 0.002) by dilution into warm (37° C.) sterile medium supplemented with identical concentrations of the inducer and antibiotic used to maintain the antisense construct. For a control, cells are also added to several wells that contain solvent used to dissolve test antibiotics but which contain no antibiotic. Cell growth is monitored continuously by incubation at 37° C. in a microtiter plate reader monitoring the OD₆₀₀ of the wells over an 18-hour period. The percent inhibition of growth produced by each concentration of antibiotic is calculated by comparing the rates of logarithmic growth against that exhibited by cells growing in medium without antibiotic. A plot of percent inhibition against log[antibiotic concentration] allows extrapolation of an IC₅₀ value for each antibiotic.

F. Determining the Specificity of the Test Antibiotics

A comparison of the IC₅₀s generated by antibiotics of known mechanism of action under antisense induced and non-induced conditions allows the pathway in which a proliferation-required nucleic acid lies to be identified. If cells expressing an antisense nucleic acid comprising a nucleotide sequence complementary to a proliferation-required gene are selectively sensitive to an antibiotic acting via a particular pathway, then the gene against which the antisense acts is involved in the pathway on which the antibiotic acts.

G. Identification of Pathway in Which a Test Antibiotic Acts

As discussed above, the cell-based assay may also be used to determine the pathway against which a test antibiotic acts. In such an analysis, the pathways against which each member of a panel of antisense nucleic acids acts are identified as described above. A panel of cells, each containing an inducible vector which transcribes a stabilized antisense nucleic acid comprising a nucleotide sequence complementary to a gene in a known proliferation-required pathway, is contacted with a test antibiotic for which it is desired to determine the pathway on which it acts under inducing and non-inducing conditions. If heightened sensitivity is observed in induced cells transcribing stabilized antisense complementary to a gene in a particular pathway but not in induced cells transcribing stabilized antisense nucleic acids comprising nucleotide sequences complementary to genes in other pathways, then the test antibiotic acts against the pathway for which heightened sensitivity was observed.

One skilled in the art will appreciate that further optimization of the assay conditions, such as the concentration of inducer used to induce antisense transcription and/or the growth conditions used for the assay (for example incubation temperature and medium components) may further increase the selectivity and/or magnitude of the antibiotic sensitization exhibited.

In some embodiments, the antisense RNA can be stabilized by expression in mutant host strains having a reduced ability to degrade RNA. In yet another embodiment, the RNA stabilization methods disclosed herein (i.e. stabilization by transcribing antisense RNAs flanked on each end by at least one stem-loop and stabilization by transcription of antisense RNA in hosts having reduced abillity to degrade RNA) can be combined for use in the above cell-based assays.

It will also be appreciated that the above cell-based assays may be performed using stabilized antisense nucleic acids complementary to any of the proliferation-required nucleic acids identified as described herein, or portions thereof, antisense nucleic acids complementary to homologous coding nucleic acids or portions thereof, or homologous antisense nucleic acids. In this way, the level or activity of a target, such as a proliferation-required polypeptide from a heterologous organism may be reduced.

A skilled artisan will appreciate that the methods for stabilizing RNA disclosed herein can also be used in organisms other than E. coli. One skilled in the art will recognize that vectors for the transcription of stabilized antisense RNA in organisms other than E. coli, including shuttle vectors, can be constructed using the techniques disclosed herein. In one embodiment, the vector described in U.S. Patent Application Serial No. 60/259,434, the disclosure of which is incorporated by reference in its entirety, may be modified to transcribe stabilized antisense RNAs. A skilled artisan will also recognize that host organisms other than E. coli that have a reduced ability to degrade RNA can be used for the stabilized expression of RNA. Furthermore, one skilled in the art will appreciate that both stabilization methods can be used together and in conjunction with the above cell-based assays.

In particular, the above cell-based assays employing methods of stabilizing RNA expression can be used with organisms including, but not limited to, Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species. In one embodiment of the current invention, these organisms include gram-negative bacteria. In some embodiments the host organism is grown with vigorous shaking to ensure homogenous distribution throughout the growth medium and thus, accurate measurement of proliferation ability.

The following example confirms the effectiveness of the methods described above.

### Example 14

### Identification of the Biological Pathway in Which a Proliferation-Required Gene Lies

The effectiveness of the above assays was validated using proliferation-required genes from E. coli that were identified using non-stabilized antisense RNAs. Antibiotics of various chemical classes and modes of action were purchased from Sigma Chemicals (St. Louis, Mo.). Stock solutions were prepared by dissolving each antibiotic in an appropriate aqueous solution based on information provided by the manufacturer. The final working solution of each antibiotic contained no more than 0.2% (w/v) of any organic solvent. To determine their potency against a bacterial strain engineered for transcription of an antisense comprising a nucleotide sequence complementary to a proliferation-required 50S ribosomal protein, each antibiotic was serially diluted two-or three-fold in growth medium supplemented with the appropriate antibiotic for maintenance of the antisense construct. At least ten dilutions were prepared for each antibiotic. 25 μL aliquots of each dilution were transferred to discrete wells of a 384-well microplate (the assay plate) using a multi-channel pipette. Quadruplicate wells were used for each dilution of an antibiotic under each treatment condition (plus and minus inducer). Each assay plate contained twenty wells for cell growth controls (growth medium replacing antibiotic), ten wells for each treatment (plus and minus inducer, in this example IPTG). Assay plates were usually divided into the two treatments: half the plate containing induced cells and an appropriate concentrations of inducer (in this example IPTG) to maintain the state of induction, the other half containing non-induced cells in the absence of IPTG.

Cells for the assay were prepared as follows. Bacterial cells containing a construct, from which transcription of antisense nucleic acid comprising a nucleotide sequence complementary to rplL and rplJ (AS-rplL/J), which encode proliferation-required 50S ribosomal subunit proteins, is inducible in the presence of IPTG, were grown into exponential growth (OD₆₀₀ 0.2 to 0.3) and then diluted 1:100 into fresh medium containing either 400 μM or 0 μM inducer (IPTG). These cultures were incubated at 37° C. for 2.5 hr. After a 2.5 hr incubation, induced and non-induced cells were respectively diluted into an assay medium at a final OD₆₀₀ value of 0.0004. The medium contained an appropriate concentration of the antibiotic for the maintenance of the antisense construct. In addition, the medium used to dilute induced cells was supplemented with 800 μM IPTG so that addition to the assay plate would result in a final IPTG concentration of 400 μM. Induced and non-induced cell suspensions were dispensed (25 μl/well) into the appropriate wells of the assay plate as discussed previously. The plate was then loaded into a plate reader, incubated at constant temperature, and cell growth was monitored in each well by the measurement of light scattering at 595 nm. Growth was monitored every 5 minutes until the cell culture attained a stationary growth phase. For each concentration of antibiotic, a percentage inhibition of growth was calculated at the time point corresponding to mid-exponential growth for the associated control wells (no antibiotic, plus or minus IPTG). For each antibiotic and condition (plus or minus IPTG), a plot of percent inhibition versus log of antibiotic concentration was generated and the IC₅₀ determined. A comparison of the IC₅₀ for each antibiotic in the presence and absence of IPTG revealed whether induction of the antisense construct sensitized the cell to the mechanism of action exhibited by the antibiotic. Cells which exhibited a statistically significant decrease in the IC₅₀ value in the presence of inducer were considered to have an increased sensitivity to the test antibiotic.

The results are provided in the table below, which lists the classes and names of the antibiotics used in the analysis, the targets of the antibiotics, the IC₅₀ in the absence of IPTG, the IC₅₀ in the presence of IPTG, the concentration units for the IC₅₀s, the fold increase in IC₅₀ in the presence of IPTG, and whether increased sensitivity was observed in the presence of IPTG.

TABLE I
Effect of Expression of Antisense RNA to rplL and rplJ on Antibiotic Sensitivity

| ANTIBIOTIC CLASS/Names | TARGET | IC50 (−IPTG) | IC50 (+IPTG) | Conc. | Fold Increase | Sensitivity |
|---|---|---|---|---|---|---|
| PROTEIN SYNTHESIS | | | | | | |
| INHIBITOR ANTIBIOTICS | | | | | | |
| AMINOGLYCOSIDES | | | | | | |
| Gentamicin | 30S ribosome function | 2715 | 19.19 | ng/ml | 141 | Yes |
| Streptomycin | 30S ribosome function | 11280 | 161 | ng/ml | 70 | Yes |
| Spectinomycin | 30S ribosome function | 18050 | <156 | ng/ml | Yes | |
| Tobramycin | 30S ribosome function | 3594 | 70.58 | ng/ml | 51 | Yes |
| MACROLIDES | | | | | | |
| Erythromycin | 50S ribosome function | 7467 | 187 | ng/ml | 40 | Yes |
| AROMATIC POYKETIDES | | | | | | |
| Tetracycline | 30S ribosome function | 199.7 | 1.83 | ng/ml | 109 | Yes |
| Minocycline | 30S ribosome function | 668.4 | 3.897 | ng/ml | 172 | Yes |
| Doxycycline | 30S ribosome function | 413.1 | 27.81 | ng/ml | 15 | Yes |
| OTHER PROTEIN SYNTHESIS INHIBITORS | | | | | | |
| Fusidic acid | Elongation Factor G function | 59990 | 641 | ng/ml | 94 | Yes |
| Chloramphenicol | 30S ribosome function | 465.4 | 1.516 | ng/ml | 307 | Yes |
| Lincomycin | 50S ribosome function | 47150 | 324.2 | ng/ml | 145 | Yes |
| OTHER ANTIBIOTIC MECHANISMS | | | | | | |
| B-LACTAMS | | | | | | |
| Cefoxitin | Cell wall biosynthesis | 2782 | 2484 | ng/ml | 1 | No |
| Cefotaxime | Cell wall biosynthesis | 24.3 | 24.16 | ng/ml | 1 | No |
| DNA SYNTHESIS INHIBITORS | | | | | | |
| Nalidixic acid | DNA Gyrase activity | 6973 | 6025 | ng/ml | 1 | No |
| Ofloxacin | DNA Gyrase activity | 49.61 | 45.89 | ng/ml | 1 | No |
| OTHER | | | | | | |
| Bacitracin | Cell membrane function | 4077 | 4677 | ng/ml | 1 | No |
| Trimethoprim | Dihydrofolate Reductase | 128.9 | 181.97 | ng/ml | 1 | No |
| | activity | | | | | |
| Vancomycin | Cell wall biosynthesis | 145400 | 72550 | ng/ml | 2 | No |

The above results demonstrate that induction of an antisense RNA to genes encoding 50S ribosomal subunit proteins results in a selective and highly significant sensitization of cells to antibiotics that inhibit ribosomal function and protein synthesis. The above results further demonstrate that induction of an antisense construct to an essential gene sensitizes an organism to compounds that interfere with that gene products' biological role. This sensitization is restricted to compounds that interfere with pathways associated with the targeted gene and it's product.

Assays utilizing antisense constructs to essential genes can be used to identify compounds that specifically interfere with the activity of multiple targets in a pathway. Such constructs can be used to simultaneously screen a sample against multiple targets in one pathway in one reaction (Combinatorial HTS).

It will be appreciated that analyses such as that described above may be performed using the stabilized antisense RNAs of the present invention. Furthermore, as discussed above, panels of cells containing constructs which can transcribe stabilized antisense RNAs may be used to characterize the point of intervention of any compound affecting an essential biological pathway including antibiotics with no known mechanism of action.

Other representative known antibiotics which may be used in the above methods are provided in the table below. However, it will be appreciated that other antibiotics may also be used.

TABLE II
Antibiotics: Mechanism of Action and Known Resistance Genes

| | | RESISTANT |
|---|---|---|
| ANTIBIOTIC | INHIBITS/TARGET | MUTANTS |
| Inhibitors of Transcription | | |
| Rifamycin, 1959 Rifampicin | Inhibits initiation of transcription/β-subunit | rpoB, crp, cyaA |
| Rifabutin Rifaximin | RNA polymerase, rpoB | |
| Streptolydigin | Accelerates transcription chain | rpoB |
| | termination/β-subunit RNA polymerase | |
| Streptovaricin | an acyclic ansamycin, inhibits RNA | rpoB |
| | polymerase | |
| Actinomycin D + EDTA | Intercalates between 2 successive G-C | pldA |
| | pairs, rpoB, inhibits RNA synthesis | |
| Inhibitors of Nucleic Acid Metabolism | | |
| Quinolones, 1962 Nalidixic | α subunit gyrase and/or topoisomerase IV, | gyrAorB, icd, sloB |
| acid Oxolmic acid | gyrA | |
| Fluoroquinolones | α subunit gyrase, gyrA and/or | gyrA |
| Ciprofloxacin, 1983 | topoisomerase IV (probable target in Staph) | norA (efflux in Staph) |
| Norfloxacin | | hipQ |
| Coumerins Novobiocin | Inhibits ATPase activity of β-subunit | gyrB, cysB, cysE, nov, |
| | gyrase, gyrB | ompA |
| Coumermycin | Inhibits ATPase activity of β-subunit | gyrB, hisW |
| | gyrase, gyrB | |
| Albicidin | DNA synthesis | tsx (nucleoside channel) |
| Metronidazole | Causes single-strand breaks in DNA | nar |
| Inhibitors of Metabolic Pathways | | |
| Sulfonamides, 1932 | blocks synthesis of dihydrofolate, dihydro- | folP, gpt, pabA, pabB, |
| Sulfanilamide | pteroate synthesis, folP | pabC |
| Trimethoprim, 1962 | Inhibits dihydrofolate reductase, folA | folA, thyA |
| Showdomycin | Nucleoside analogue capable of alkylating | nupC, pnp |
| | sulfhydryl groups, inhibitor of thymidylate | |
| | synthetase | |
| Thiolactomycin | type II fatty acid synthase inhibitor | emrB |
| | | fadB, emrB due to gene |
| | | dosage |
| Psicofuranine | Adenosine glycoside antibiotic, target is | guaA, B |
| | GMP synthetase | |
| Triclosan | Inhibits fatty acid synthesis | fabI (envM) |
| Diazoborines Isoniazid, | heterocyclic, contains boron, inhibit fatty | fabI (envM) |
| Ethionamide | acid synthesis, enoyl-ACP reductase, fabI | |
| Inhibitors of Translation | | |
| Phenylpropanoids | Binds to ribosomal peptidyl transfer center | rrn, cmlA, marA, ompF, |
| Chloramphenicol, 1947 | preventing peptide translocation/binds to | ompR |
| | S6, L3, L6, L14, L16, L25, L26, L27, but | |
| | preferentially to L16 | |
| Tetracyclines, 1948, type II | Binding to 30S ribosomal subunit, “A” site | clmA (cmr), mar, ompF |
| polyketides | on 30S subunit, blocks peptide elongation, | |
| Minocycline | strongest binding to S7 | |
| Doxycycline | | |
| Macrolides (type I polyketides) | Binding to 50 S ribosomal subunit, 23S | rrn, rplC, rplD, rplV, |
| Erythromycin, 1950 | rRNA, blocks peptide translocation, L15, | mac |
| Carbomycin, Spiramycin | L4, L12 | |
| etc | | |
| Aminoglycosides Streptomycin, | Irreversible binding to 30S ribosomal | rpsL, strC, M, ubiF |
| 1944 | subunit, prevents translation or causes | atpA-E, ecfB, |
| Neomycin | mistranslation of mRNA/16S rRNA | hemAC, D, E, G, topA, |
| Spectinomycin | | rpsC, D, E, rrn, spcB |
| | | atpA-atpE, cpxA, ecfB, |
| Kanamycin | | hemA, B, L, topA |
| Kasugamycin | | ksgA, B, C, D, rplB, K, |
| Gentamicin, 1963 | | rpsI, N, M, R |
| Amikacin | | rplF, ubiF |
| Paromycin | | cpxA |
| | | rpsL |
| Lincosamides | Binding to 50 S ribosomal subunit, blocks | linB, rplN, O, rpsG |
| Lincomycin, 1955 | peptide translocation | |
| Clindamycin | | |
| Streptogramins Virginiamycin, | 2 components, Streptogramins A&B, bind | |
| 1955 Pristinamycin | to the 505 ribosomal subunit blocking | |
| Synercid: quinupristin/ | peptide translocation and peptide bond | |
| dalfopristin | formation | |
| Fusidanes | Inhibition of elongation factor G (EF-G) | fusA |
| Fusidic Acid | prevents peptide translocation | |
| Kirromycin (Mocimycin) | Inhibition of elongation factor TU (EF-Tu), | tufA, B |
| | prevents peptide bond formation | |
| Pulvomycin | Binds to and inhibits EF-TU | |
| Thiopeptin | Sulfur-containing antibiotic, inhibits protein | rplE |
| | synthesis, EF-G | |
| Tiamulin | Inhibits protein synthesis | rplC, rplD |
| Negamycin | Inhibits termination process of protein | prfB |
| | synthesis | |
| Oxazolidinones Linezolid | 235 rRNA | |
| Isoniazid | | |
| Nitrofurantoin | Inhibits protein synthesis, nitroreductases | pdx |
| | convert nitrofurantoin to highly reactive | nfnA, B |
| | electrophilic intermediates which attack | |
| | bacterial ribosomal proteins non- | |
| | specifically | |
| Pseudomonic Acids Mupirocin | Inhibition of isoleucyl tRNA synthetase- | ileS |
| (Bactroban) | used for Staph, topical cream, nasal | |
| | spray | |
| Indolmycin | Inhibits tryptophanyl-tRNA synthetase | trpS |
| Viomycin | | rrmA (23S rRNA |
| | | methyltransferase; |
| | | mutant has slow |
| | | growth rate, slow |
| | | chain elongation rate, |
| | | and viomycin |
| | | resistance) |
| Thiopeptides | Binds to L11-23S RNA complex | |
| Thiostrepton | Inhibits GTP hydrolysis by EF-G | |
| Micrococcin | Stimulates GTP hydrolysis by EF-G | |

In some embodiments, the antisense RNA can be stabilized by expression in mutant host strains having a reduced ability to degrade RNA. In yet another embodiment, the RNA stabilization methods disclosed herein (i.e. stabilization by transcribing antisense RNAs flanked on each end by at least one stem-loop and stabilization by transcription of antisense RNA in hosts having reduced abillity to degrade RNA) can be combined for use in the above cell-based assays.

It will also be appreciated that the above cell-based assays may be performed using stabilized antisense nucleic acids complementary to any of the proliferation-required nucleic acids identified as described herein, or portions thereof, antisense nucleic acids complementary to homologous coding nucleic acids or portions thereof, or homologous antisense nucleic acids. In this way, the level or activity of a target, such as a proliferation-required polypeptide from a heterologous organism may be reduced.

A skilled artisan will appreciate that the methods for stabilizing RNA disclosed herein can also be used in organisms other than E. coli. One skilled in the art will recognize that vectors for the transcription of stabilized antisense RNA in organisms other than E. coli, including shuttle vectors, can be constructed using the techniques disclosed herein. In one embodiment, the vector described in U.S. Patent Application Serial No. 60/259,434, the disclosure of which is incorporated by reference in its entirety, may be modified to transcribe stabilized antisense RNAs. A skilled artisan will also recognize that host organisms other than E. coli that have a reduced ability to degrade RNA can be used for the stabilized expression of RNA. Furthermore, one skilled in the art will appreciate that both stabilization methods can be used together and in conjunction with the above cell-based assays.

In particular, the above cell-based assays employing methods of stabilizing RNA expression can be used with organisms including, but not limited to, Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species. In one embodiment of the current invention, these organisms include gram-negative bacteria. In some embodiments the host organism is grown with vigorous shaking to ensure homogenous distribution throughout the growth medium and thus, accurate measurement of proliferation ability.

### Example 15

### Transfer of Exogenous Nucleic Acid Sequences to other Bacterial Species

The ability of an antisense molecule identified in a first organism to inhibit the proliferation of a second organism (thereby confirming that a gene in the second organism which is homologous to the gene from the first organism is required for proliferation of the second organism) was validated using non-stabilized antisense nucleic acids which inhibit the growth of E. coli which were identified using methods similar to those described above. Expression vectors which inhibited growth of E. coli upon induction of antisense RNA expression with IPTG were transformed directly into Enterobacter cloacae, Klebsiella pneumonia or Salmonella typhimurium. The transformed cells were then assayed for growth inhibition according to the methods described in Example 6. After growth in liquid culture, cells were plated at various serial dilutions and a score determined by calculating the log difference in growth for INDUCED vs. UNINDUCED antisense RNA expression as determined by the maximum 10-fold dilution at which a colony was observed. The results of these experiments are listed below in Table III. If there was no effect of antisense RNA expression in a microorganism, the clone is minus in Table III. In contrast, a positive in Table III means that at least 10 fold more cells were required to observe a colony on the induced plate than on the non-induced plate under the conditions used and in that microorganism.

TABLE III
Sensitivity of Other Microorganisms to Antisense Nucleic Acids
That Inhibit Proliferation in E. coli

| Mol. No. | S. typhimurium | E. cloacae | K. pneumoniae |
|---|---|---|---|
| EcXA001 | + | + | − |
| EcXA004 | + | − | − |
| EcXA005 | + | + | + |
| EcXA006 | − | − | − |
| EcXA007 | − | + | − |
| EcXA008 | + | − | + |
| EcXA009 | − | − | − |
| EcXA010 | + | + | + |
| EcXA011 | − | + | − |
| EcXA012 | − | + | − |
| EcXA013 | + | + | + |
| EcXA014 | + | + | − |
| EcXA015 | + | + | + |
| EcXA016 | + | + | + |
| EcXA017 | + | + | + |
| EcXA018 | + | + | + |
| EcXA019 | + | + | + |
| EcXA020 | + | + | + |
| EcXA021 | + | + | + |
| EcXA023 | + | + | + |
| EcXA024 | + | − | + |
| EcXA025 | − | − | − |
| EcXA026 | + | + | − |
| EcXA027 | + | + | − |
| EcXA028 | + | − | − |
| EcXA029 | − | − | − |
| EcXA030 | + | + | + |
| EcXA031 | + | − | − |
| EcXA032 | + | + | − |
| EcXA033 | + | + | + |
| EcXA034 | + | + | + |
| EcXA035 | − | − | − |
| EcXA036 | + | − | + |
| EcXA037 | + | + | − |
| EcXA038 | + | + | + |
| EcXA039 | + | − | − |
| EcXA041 | + | + | + |
| EcXA042 | − | + | + |
| EcXA043 | − | − | − |
| EcXA044 | − | − | − |
| EcXA045 | + | + | + |
| EcXA046 | − | − | − |
| EcXA047 | + | + | − |
| EcXA048 | − | − | − |
| EcXA049 | + | − | − |
| EcXA050 | − | − | − |
| EcXA051 | + | − | − |
| EcXA052 | + | − | − |
| EcXA053 | + | + | + |
| EcXA054 | − | − | + |
| EcXA055 | + | − | − |
| EcXA056 | + | − | + |
| EcXA057 | + | + | − |
| EcXA058 | − | − | − |
| EcXA059 | + | + | + |
| EcXA060 | − | − | − |
| EcXA061 | − | − | − |
| EcXA062 | − | − | − |
| EcXA063 | + | + | − |
| EcXA064 | − | − | − |
| EcXA065 | + | + | − |
| EcXA066 | − | − | − |
| EcXA067 | − | + | − |
| EcXA068 | − | − | − |
| EcXA069 | − | + | − |
| EcXA070 | − | − | − |
| EcXA071 | + | − | − |
| EcXA072 | + | − | + |
| EcXA073 | + | + | + |
| EcXA074 | + | + | + |
| EcXA075 | + | − | − |
| EcXA076 | − | + | − |
| EcXA077 | + | + | − |
| EcXA079 | + | + | + |
| EcXA080 | + | − | − |
| EcXA082 | − | + | − |
| EcXA083 | − | − | − |
| EcXA084 | − | + | − |
| EcXA086 | − | − | − |
| EcXA087 | − | − | − |
| EcXA088 | − | − | − |
| EcXA089 | − | − | − |
| EcXA090 | − | − | − |
| EcXA091 | − | − | − |
| EcXA092 | − | − | − |
| EcXA093 | − | − | − |
| EcXA094 | + | + | + |
| EcXA095 | + | + | − |
| EcXA096 | − | − | − |
| EcXA097 | + | − | − |
| EcXA098 | + | − | − |
| EcXA099 | − | − | − |
| EcXA100 | − | − | − |
| EcXA101 | − | − | − |
| EcXA102 | − | − | − |
| EcXA103 | − | + | − |
| EcXA104 | + | + | + |
| EcXA106 | + | + | − |
| EcXA107 | − | − | − |
| EcXA108 | − | − | − |
| EcXA109 | − | − | − |
| EcXA110 | + | + | − |
| EcXA111 | − | − | − |
| EcXA112 | − | + | − |
| EcXA113 | + | + | + |
| EcXA114 | − | + | − |
| EcXA115 | − | + | − |
| EcXA116 | + | + | − |
| EcXA117 | + | − | − |
| EcXA118 | − | − | − |
| EcXA119 | + | + | − |
| EcXA120 | − | − | − |
| EcXA121 | − | − | − |
| EcXA122 | + | − | + |
| EcXA123 | + | − | − |
| EcXA124 | − | − | − |
| EcXA125 | − | − | − |
| EcXA126 | − | − | − |
| EcXA127 | + | + | − |
| EcXA128 | − | − | − |
| EcXA129 | − | + | − |
| EcXA130 | + | + | − |
| EcXA132 | − | − | − |
| EcXA133 | − | − | − |
| EcXA136 | − | − | − |
| EcXA137 | − | − | − |
| EcXA138 | + | − | − |
| EcXA139 | − | − | − |
| EcXA140 | + | − | − |
| EcXA141 | + | − | − |
| EcXA142 | − | − | − |
| EcXA143 | − | + | − |
| EcXA144 | + | + | − |
| EcXA145 | − | − | − |
| EcXA146 | − | − | − |
| EcXA147 | − | − | − |
| EcXA148 | − | − | − |
| EcXA149 | + | + | + |
| EcXA150 | − | − | − |
| EcXA151 | + | − | − |
| EcXA152 | − | − | − |
| EcXA153 | + | + | − |
| EcXA154 | − | − | − |
| EcXA155 | − | − | ND |
| EcXA156 | − | + | − |
| EcXA157 | − | − | − |
| EcXA158 | − | − | − |
| EcXA159 | + | − | − |
| EcXA160 | + | − | − |
| EcXA162 | − | − | − |
| EcXA163 | − | − | − |
| EcXA164 | − | − | − |
| EcXA165 | − | − | − |
| EcXA166 | − | − | − |
| EcXA167 | − | − | − |
| EcXA168 | − | − | − |
| EcXA169 | − | + | − |
| EcXA171 | − | − | − |
| EcXA172 | − | − | − |
| EcXA173 | − | − | − |
| EcXA174 | − | − | − |
| EcXA175 | − | − | − |
| EcXA176 | − | − | − |
| EcXA178 | − | − | − |
| EcXA179 | − | − | − |
| EcXA180 | + | − | − |
| EcXA181 | − | − | − |
| EcXA182 | − | − | − |
| EcXA183 | − | − | − |
| EcXA184 | − | − | − |
| EcXA185 | − | − | − |
| EcXA186 | − | − | − |
| EcXA187 | + | + | + |
| EcXA189 | + | − | − |
| EcXA190 | + | + | + |
| EcXA191 | + | + | − |
| EcXA192 | − | + | − |

In some embodiments, the antisense RNA can be stabilized by expression in mutant host strains having a reduced ability to degrade RNA. In yet another embodiment, the RNA stabilization methods disclosed herein (i.e. stabilization by transcribing antisense RNAs flanked on each end by at least one stem-loop and stabilization by transcription of antisense RNA in hosts having reduced abillity to degrade RNA) can be combined for use in the above cell-based assays.

It will also be appreciated that the above cell-based assays may be performed using stabilized antisense nucleic acids complementary to any of the proliferation-required nucleic acids identified as described herein, or portions thereof, antisense nucleic acids complementary to homologous coding nucleic acids or portions thereof, or homologous antisense nucleic acids. In this way, the level or activity of a target, such as a proliferation-required polypeptide from a heterologous organism may be reduced.

A skilled artisan will appreciate that the methods for stabilizing RNA disclosed herein can also be used in organisms other than E. coli. One skilled in the art will recognize that vectors for the transcription of stabilized antisense RNA in organisms other than E. coli, including shuttle vectors, can be constructed using the techniques disclosed herein. In one embodiment, the vector described in U.S. patent application Ser. No. 60/259,434, the disclosure of which is incorporated by reference in its entirety, may be modified to transcribe stabilized antisense RNAs. A skilled artisan will also recognize that host organisms other than E. coli that have a reduced ability to degrade RNA can be used for the stabilized expression of RNA. Furthermore, one skilled in the art will appreciate that both stabilization methods can be used together and in conjunction with the above cell-based assays.

In particular, the above methods for evaluating the ability of stabilized antisense RNA to inhibit the proliferation of a heterologous organism can be used with organisms including, but not limited to, Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species. In one embodiment of the current invention, these organisms include gram-negative bacteria. In some embodiments the host organism is grown with vigorous shaking to ensure homogenous distribution throughout the growth medium and thus, accurate measurement of proliferation ability.

Those skilled in the art will appreciate that a negative result in a heterologous cell or microorganism does not mean that that cell or microorganism is missing that gene nor does it mean that the gene is unessential. However, a positive result means that the heterologous cell or microorganism contains a homologous gene which is required for proliferation of that cell or microorganism. The homologous gene may be obtained using the methods described herein. Those cells that are inhibited by antisense may be used in cell-based assays as described herein for the identification and characterization of compounds in order to develop antibiotics effective in these cells or microorganisms. Those skilled in the art will appreciate that an antisense molecule which works in the microorganism from which it was obtained will not always work in a heterologous cell or microorganism.

All documents cited herein are incorporated herein by reference in their entireties.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit and scope of that which is described and claimed.

### Example 16

### Identification of Sequences Homologous to Proliferation-required Genes

As a demonstration of the methodology required to find homologues to an essential gene, homologs of essential genes from several different microorganism which were identified using non-stabilized antisense transcripts were identified as follows. First, the most reliable source of gene sequences for each organism was assessed by conducting a survey of the public and private data sources. The nine organisms studied were Enterococcus faecalis, Escherichia coli, Haemophilus influenzae, Helicobacter pylori, Klebsiella pneumoniae, Pseudomonas aeruginosa, Staphylococcus aureus, Streptococcus pneumoniae and Salmonella typhi. Full-length gene protein and nucleotide sequences for these organisms were assembled from various sources. For Escherichia coli, Haemophilus influenzae and Helicobacter pylori, gene sequences were adopted from the public sequencing projects, and derived from the GenPept 115 database (available from NCBI). For Pseudomonas aeruginosa, gene sequences were adopted from the Pseudomonas genome sequencing project (downloaded from http://www.pseudomonas.com). For Klebsiella pneumoniae, Staphylococcus aureus, Streptococcus pneumoniae and Salmonella typhi, genomic sequences from PathoSeq v 4.1 (March 2000 release) was reanalyzed for ORFs using the gene finding software GeneMark v 2.4a, which was purchased from GenePro Inc. 451 Bishop St., N.W., Suite B, Atlanta, Ga., 30318, USA.

The genes identified as being essential were compared to genes from other organisms using the FASTA program v3.3. Genes were considered homologues if they were greater than 25% identical and the alignment between the two genes covered more than 70% of the length of one of the genes. The best homologue for each of the nine organisms, defined as the most significantly scoring match which also fulfilled the above criteria was reported. For many of the proliferation-required gene tested homologues were found in every heterologous organism examined.

It will be appreciated that similar analyses may be conducted using genes identified using stabilized antisense nucleic acids.

Use of Isolated Exogenous Nucleic Acid Fragments as Antisense Antibiotics

In addition to using stabilzed antisense nucleic acids identified as described herein to enable screening of molecule libraries to identify compounds useful to identify antibiotics, stabilized antisense nucleic acids complementary to the proliferation-required sequences or portions thereof, stabilized antisense nucleic acids complementary to homologous coding nucleic acids, or stabilized homologous antisense nucleic acids can be used as therapeutic agents. Specifically, the proliferation-required sequences or homolgous coding nucleic acids, or portions therof, can be provided to an individual as stabilized antisense nucleic acids or stabilized homologous antisense nucleic acids to inhibit the translation of a bacterial target gene or the processing, folding, or assembly into a protein/RNA complex of a nontranslated RNA.

### Example 17

### Generation of Antisense Therapeutics from Identified Exogenous Sequences

Stabilized antisense nucleic acids complementary to proliferation-required nucleic acid sequences identified as described herein, or portions thereof, stabilized antisense nucleic acids complementary to homologous coding nucleic acids, or portions thereof, or stabilized homologous antisense nucleic acids or portions thereof can be used as antisense therapeutics for the treatment of bacterial infections or simply for inhibition of bacterial growth in vitro or in vivo. For example, stabilized antisense nucleic acid therapeutics, which are flanked on each end by at least one stem-loop structure, may be used to treat infections caused by or inhibit the growth of Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species.

The therapy exploits the biological process in cells where genes are transcribed into messenger RNA (mRNA) that is then translated into proteins. Stabilized antisense RNA technology contemplates the use of stabilized antisense nucleic acids, including stabilized antisense oligonucleotides, complementary to a target gene that will bind to its target nucleic acid and decrease or inhibit the expression of the target gene. For example, the stabilized antisense nucleic acid may inhibit the translation or transcription of the target nucleic acid. In one embodiment, stabilized antisense oligonucleotides can be used to treat and control a bacterial infection of a cell culture containing a population of desired cells contaminated with bacteria. In another embodiment, the stabilized antisense oligonucleotides can be used to treat an organism with a bacterial infection.

Stabilized antisense oligonucleotides, which are flanked on each end by at least one stem-loop structure, can be synthesized from any of the nucleic acid sequences identified as described herein by using methods well known in the art. In a preferred embodiment, stabilized antisense oligonucleotides are synthesized using artificial means. Uhlmann & Peymann, Chemical Rev. 90:543-584 (1990) review antisense oligonucleotide technology in detail. Modified or unmodified stabilized antisense oligonucleotides can be used as therapeutic agents. Modified stabilized antisense oligonucleotides are preferred. Modification of the phosphate backbones of the stabilized antisense oligonucleotides can be achieved by substituting the internucleotide phosphate residues with methylphosphonates, phosphorothioates, phosphoramidates, and phosphate esters. Nonphosphate internucleotide analogs such as siloxane bridges, carbonate brides, thioester bridges, as well as many others known in the art may also be used. The preparation of certain antisense oligonucleotides with modified internucleotide linkages is described in U.S. Pat. No. 5,142,047, hereby incorporated by reference. Such oligonucleotides can be modified to include a stem-loop structure at one or preferably both ends.

Modifications to the nucleoside units of the stabilized antisense oligonucleotides are also contemplated. These modifications can further increase the half-life and increase cellular rates of uptake for the oligonucleotides in vivo. For example, α-anomeric nucleotide units and modified nucleotides such as 1,2-dideoxy-d-ribofuranose, 1,2-dideoxy-1-phenylribofuranose, and N⁴, N⁴-ethano-5-methyl-cytosine are contemplated for use in the present invention.

An additional form of modified stabilized antisense molecules is found in peptide nucleic acids. Peptide nucleic acids (PNA) have been developed to hybridize to single and double stranded nucleic acids. PNA are nucleic acid analogs in which the entire deoxyribose-phosphate backbone has been exchanged with a chemically different, but structurally homologous, polyamide (peptide) backbone containing 2-aminoethyl glycine units. Unlike DNA, which is highly negatively charged, the PNA backbone is neutral. Therefore, there is much less repulsive energy between complementary strands in a PNA-DNA hybrid than in the comparable DNA-DNA hybrid, and consequently they are much more stable. PNA can hybridize to DNA in either a Watson/Crick or Hoogsteen fashion (Demidov et al., Proc. Natl. Acad. Sci. USA. 92:2637-2641, 1995; Egholm, Nature 365:566-568, 1993; Nielsen et al., Science 254:1497-1500, 1991; Dueholm et al., New J. Chem. 21:19-31, 1997).

Molecules called PNA “clamps” have been synthesized which have two identical PNA sequences joined by a flexible hairpin linker containing three 8-amino-3,6-dioxaoctanoic acid units. When a PNA clamp is mixed with a complementary homopurine or homopyrimidine DNA target sequence, a PNA-DNA-PNA triplex hybrid can form which has been shown to be extremely stable (Bentin et al., Biochemistry 35:8863-8869, 1996; Eghohm et al., Nucleic Acids Res. 23:217-222, 1995; Griffith et al., J. Am. Chem. Soc. 117:831-832, 1995).

The sequence-specific and high affinity duplex and triplex binding of PNA have been extensively described (Nielsen et al., Science 254:1497-1500, 1991; Egholm et al., J. Am. Chem. Soc. 114:9677-9678, 1992; Egholm et al., Nature 365:566-568, 1993; Almarsson et al., Proc. Natl. Acad. Sci. U.S.A. 90:9542-9546, 1993; Demidov et al., Proc. Natl. Acad. Sci. U.S.A. 92:2637-2641, 1995). They have also been shown to be resistant to nuclease and protease digestion (Demidov et al., Biochem. Pharm. 48:1010-1313, 1994). PNA has been used to inhibit gene expression (Hanvey et al., Science 258:1481-1485,1992; Nielsen et al., Nucl. Acids. Res., 21:197-200, 1993; Nielsen et al., Gene 149:139-145, 1994; Good & Nielsen, Science, 95: 2073-2076, 1998; all of which are hereby incorporated by reference), to block restriction enzyme activity (Nielsen et al., supra., 1993), to act as an artificial transcription promoter (Mollegaard, Proc. Natl. Acad. Sci. U.S.A. 91:3892-3895, 1994) and as a pseudo restriction endonuclease (Demidov et al., Nucl. Acids. Res. 21:2103-2107, 1993). Recently, PNA has also been shown to have antiviral and antitumoral activity mediated through an antisense mechanism (Norton, Nature Biotechnol., 14:615-619, 1996; Hirschman et al., J. Investig. Med. 44:347-351, 1996). PNAs have been linked to various peptides in order to promote PNA entry into cells (Basu et al., Bioconj. Chem. 8:481-488, 1997; Pardridge et al., Proc. Natl. Acad. Sci. U.S.A. 92:5592-5596, 1995).

The stabilized antisense oligonucleotides, which are flanked on each end by at least one stem-loop structure, contemplated by the present invention can be administered by direct application of oligonucleotides to a target using standard techniques well known in the art. The stabilized antisense oligonucleotides can be generated within the target using a plasmid, or a phage. In particular, the plasmid consturcts described herein may be used. Alternatively, the stabilized antisense nucleic acid may be expressed from a nucleotide sequence in the chromosome of the target cell. For example, a promoter may be introduced into the chromosome of the target cell near the target gene such that the promoter directs the transcription of the stabilized antisense nucleic acid. Alternatively, a nucleic acid containing the stabilized antisense sequence operably linked to a promoter may be introduced into the chromosome of the target cell. It is further contemplated that the stabilized antisense oligonucleotides are incorporated in a ribozyme sequence to enable the stabilized antisense to specifically bind and cleave its target mRNA. For technical applications of ribozyme and antisense oligonucleotides see Rossi et al., Pharmacol. Ther. 50(2):245-254, (1991), which is hereby incorporated by reference. The present invention also contemplates using a retron to introduce an stabilized antisense oligonucleotide to a cell. Retron technology is exemplified by U.S. Pat. No. 5,405,775, which is hereby incorporated by reference. Stabilized antisense oligonucleotides can also be delivered using liposomes or by electroporation techniques which are well known in the art.

The stabilized antisense nucleic acids identified as described herein can also be used to design antibiotic compounds comprising nucleic acids which function by intracellular triple helix formation. Triple helix oligonucleotides are used to inhibit transcription from a genome. The stabiliized antisense nucleic acids can be used to inhibit cell or microorganism gene expression in individuals infected with such microorganisms or containing such cells. Traditionally, homopurine sequences were considered the most useful for triple helix strategies. However, homopyrimidine sequences can also inhibit gene expression. Such homopyrimidine oligonucleotides bind to the major groove at homopurine:homopyrimidine sequences. Thus, both types of sequences based on the sequences identified as described herein or homologous nucleic acids that are required for proliferation are contemplated for use as antibiotic compound templates.

The stabilized antisense nucleic acids, such as stabilized antisense oligonucleotides, which are complementary to the proliferation-required nucleic acids identified as described herein or to homologous coding nucleic acids, or portions thereof, may be used to induce bacterial cell death or at least bacterial stasis by inhibiting target nucleic acid transcription or translation. Stabilized antisense oligonucleotides complementary to about 8 to 40 nucleotides of the proliferation-required nucleic acids identified as described herein or homologous coding nucleic acids have sufficient complementarity to form a duplex with the target sequence under physiological conditions.

To kill bacterial cells or inhibit their growth, the stabilized antisense oligonucleotides are applied to the bacteria or to the target cells under conditions that facilitate their uptake. These conditions include sufficient incubation times of cells and oligonucleotides so that the stabilized antisense oligonucleotides are taken up by the cells. In one embodiment, an incubation period of 7-10 days is sufficient to kill bacteria in a sample. An optimum concentration of stabilized antisense oligonucleotides is selected for use.

The concentration of stabilized antisense oligonucleotides to be used can vary depending on the type of bacteria sought to be controlled, the nature of the stabilized antisense oligonucleotide to be used, and the relative toxicity of the stabilized antisense oligonucleotide to the desired cells in the treated culture. Stabilized antisense oligonucleotides can be introduced to cell samples at a number of different concentrations preferably between 1×10⁻¹⁰M to 1×10⁻⁴M. Once the minimum concentration that can adequately control gene expression is identified, the optimized dose is translated into a dosage suitable for use in vivo. For example, an inhibiting concentration in culture of 1×10⁻⁷ translates into a dose of approximately 0.6 mg/kg body weight. Levels of oligonucleotide approaching 100 mg/kg body weight or higher may be possible after testing the toxicity of the oligonucleotide in laboratory animals. It is additionally contemplated that cells from the subject are removed, treated with the stabilized antisense oligonucleotide, and reintroduced into the subject. This range is merely illustrative and one of skill in the art are able to determine the optimal concentration to be used in a given case.

After the bacterial cells have been killed or controlled in a desired culture, the desired cell population may be used for other purposes.

### Example 18

### Use of Antisense Oligonucleotides to Treat Contaminated Cell Cultures

The following example demonstrates the ability of a stabilized antisense oligonucleotide or a stabilized antisense oligonucleotide complementary to a homologous coding nucleic acid, or portions thereof, to act as a bacteriocidal or bacteriostatic agent to treat a contaminated cell culture system. The application of the stabilized antisense oligonucleotides is thought to inhibit the translation of bacterial gene products required for proliferation. The stabilized antisense nucleic acids may also inhibit the transcription, folding or processing of the target RNA.

In one embodiment of the present invention, the stabilized antisense oligonucleotide, which is flanked on each end by at least one stem-loop structure, may comprise a phosphorothioate modified nucleic acid comprising at least about 15, at least about 20, at least about 25, at least about 30, at least about 35, at least about 40, or more than 40 consecutive nucleotides of a stabilized antisense nucleic acid identified as described herein. A sense oligodeoxynucleotide, which is flanked on each end by at least one stem-loop structure, complementary to the stabilized antisense sequence is synthesized and used as a control. The oligonucleotides are synthesized and purified according to the procedures of Matsukura, et al., Gene 72:343 (1988). The test oligonucleotides are dissolved in a small volume of autoclaved water and added to culture medium to make a 100 micromolar stock solution.

Human bone marrow cells are obtained from the peripheral blood of two patients and cultured according standard procedures well known in the art. The culture is innoculated with an organism such as Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis, any species falling within the genera of any of the above species or an organism containing a homologous nucleic acid. The culture is then incubated at 37° C. overnight to establish bacterial infection.

The stabilized control and stabilized antisense oligonucleotide containing solutions are added to the contaminated cultures and monitored for bacterial growth. After a 10 hour incubation of culture and oligonucleotides, samples from the control and experimental cultures are drawn and analyzed for the translation of the target bacterial gene using standard microbiological techniques well known in the art. The target Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species gene or an organism containing the homologous coding nucleic acid is found to be translated in the control culture treated with the stabilized control oligonucleotide, however, translation of the target gene in the experimental culture treated with the stabilized antisense oligonucleotide of the present invention is not detected or reduced, indicating that the culture is no longer contaminated or is contaminated at a reduced level.

### Example 19

### Use of Antisense Oligonucleotides to Treat Infections

A subject suffering from a Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis or any species falling within the genera of any of the above species infection or an infection with an organism containing a homologous coding nucleic acid is treated with the stabilized antisense oligonucleotide preparation above. The stabilized antisense oligonucleotide, which is flanked on each end by at least one stem-loop structure, is provided in a pharmaceutically acceptable carrier at a concentration effective to inhibit the transcription or translation of the target nucleic acid. The present subject is treated with a concentration of stabilized antisense oligonucleotide sufficient to achieve a blood concentration of about 0.1-100 micromolar. The patient receives daily injections of stabilized antisense oligonucleotide to maintain this concentration for a period of 1 week. At the end of the week a blood sample is drawn and analyzed for the presence or absence of the organism using standard techniques well known in the art. There is no detectable evidence of Anaplasma marginale, Aspergillus fumigatus, Bacillus anthracis, Bacteroides fragilis, Bordetella pertussis, Burkholderia cepacia, Campylobacter jejuni, Candida albicans, Candida glabrata (also called Torulopsis glabrata), Candida tropicalis, Candida parapsilosis, Candida guilliermondii, Candida krusei, Candida kefyr (also called Candida pseudotropicalis), Candida dubliniensis, Chlamydia pneumoniae, Chlamydia trachomatus, Clostridium botulinum, Clostridium difficile, Clostridium perfringens, Coccidioides immitis, Corynebacterium diptheriae, Cryptococcus neoformans, Enterobacter cloacae, Enterococcus faecalis, Enterococcus faecium, Escherichia coli, Haemophilus influenzae, Helicobacter pylori, Histoplasma capsulatum, Klebsiella pneumoniae, Listeria monocytogenes, Mycobacterium leprae, Mycobacterium tuberculosis, Neisseria gonorrhoeae, Neisseria meningitidis, Nocardia asteroides, Pasteurella haemolytica, Pasteurella multocida, Pneumocystis carinii, Proteus vulgaris, Pseudomonas aeruginosa, Salmonella bongori, Salmonella cholerasuis, Salmonella enterica, Salmonella paratyphi, Salmonella typhi, Salmonella typhimurium, Staphylococcus aureus, Listeria monocytogenes, Moxarella catarrhalis, Shigella boydii, Shigella dysenteriae, Shigella flexneri, Shigella sonnei, Staphylococcus epidermidis, Streptococcus pneumoniae, Streptococcus mutans, Treponema pallidum, Yersinia enterocolitica, Yersinia pestis, any species falling within the genera of any of the above species or an organim containing a homologous coding nucleic acid and the treatment is terminated.

Stabilized antisense nucleic acids complementary to a homologous coding nucleic acid or a portion thereof may be used in the preceding method to treat individuals infected with an organism containing the homologous coding nucleic acid.

### Example 20

### Preparation and Use of Triple Helix Forming Oligonucleotides

The sequences of proliferation-required nucleic acids, homologous coding nucleic acids, or homologous antisense nucleic acids are scanned to identify 10-mer to 20-mer homopyrimidine or homopurine stretches that could be used in triple-helix based strategies for inhibiting gene expression. Following identification of candidate homopyrimidine or homopurine stretches, their efficiency in inhibiting gene expression is assessed by introducing varying amounts of stabilized oligonucleotides containing the candidate sequences into a population of bacterial cells that normally express the target gene. The stabilized oligonucleotides may be prepared on an oligonucleotide synthesizer or they may be purchased commercially from a company specializing in custom oligonucleotide synthesis.

The stabilized oligonucleotides, which are flanked on each end by at least one stem-loop structure, can be introduced into the cells using a variety of methods known to those skilled in the art, including but not limited to calcium phosphate precipitation, DEAE-Dextran, electroporation, liposome-mediated transfection or native uptake.

Treated cells are monitored for a reduction in proliferation using techniques such as monitoring growth levels as compared to untreated cells using optical density measurements. The stabilized oligonucleotides that are effective in inhibiting gene expression in cultured cells can then be introduced in vivo using the techniques well known in that art at a dosage level shown to be effective.

In some embodiments, the natural (beta) anomers of the stabilized oligonucleotide units can be replaced with alpha anomers to render the stabilized oligonucleotide even more resistant to nucleases. Further, an intercalating agent such as ethidium bromide, or the like, can be attached to the 3′ end of the alpha oligonucleotide to stabilize the triple helix. For information on the generation of oligonucleotides suitable for triple helix formation see Griffin et al. (Science 245:967-971 (1989), which is hereby incorporated by this reference).

What is claimed is:

1. A method for screening a candidate antibiotic compound which inhibits the proliferation of a cell said method comprising the steps of:
(a) sensitizing a cell by providing a sublethal level of an antisense nucleic acid complementary to at least a portion of a gene encoding a proliferation-required gene product in said cell, wherein said antisense nucleic acid is flanked on each end by at least one stem-loop structure;
(b) contacting said sensitized cell with a candidate antibiotic compound; and
(c) determining the degree to which said candidate antibiotic compound inhibits proliferation of said sensitized cell relative to a cell which has not been sensitized.

2. The method of claim 1, wherein said at least one stem-loop structure formed at the 5′ end of said antisense nucleic acid comprises a flush, double stranded 5′ end.

3. The method of claim 1, wherein the activity of at least one enzyme involved in RNA degradation has been reduced in said sensitized cell.

4. The method of claim 3, wherein said at least one enzyme involved in RNA degradation is selected from the group consisting of RNase E, RNase II, RNase III, polynucleotide phosphorylase, and poly(A) polymerase.

5. The method of claim 1, wherein said step of sensitizing said cell comprises transcribing said antisense nucleic acid from a promoter.

6. The method of claim 5, wherein said promoter is regulatable.

7. The method of claim 5, wherein the first transcribed nucleotide from said promoter is the first nucleotide of a 5′ stem-loop structure.

8. The method of claim 1, wherein said at least one stem-loop structure comprises SEQ ID NO.: 5.

9. The method of claim 1, wherein said antisense nucleic acid lacks RNase E recognition sites.

10. The method of claim 1, wherein said at least one stem-loop structure lacks RNase III recognition sites.

11. The method of claim 1, wherein said at least one stem-loop structure lacks a ribosome binding site.

12. The method of claim 1, wherein said at least one stem-loop structure formed at the 3′ end of said antisense nucleic acid comprises at least one rho independent terminator.

13. The method of claim 1, wherein said sensitized cell is a gram-negative bacterium.

14. The method of claim 1, wherein said sensitized cell is selected from a group consisting of Bacteroides fragilis, Bordetella pertussis, Burkholderia cepacia, Campylobacter jejuni, Chlamydia pneumoniae, Chlamydia trachomatus, Enterobacter cloacae, Escherichia coli, Haemophilus influenzae, Helicobacter pylori, Klebsiella pneumoniae, Neisseria gonorrhoeae, Neisseria meningitidis, Pasteurella haemolytica, Pasteurella multocida, Proteus vulgaris, Pseudomonas aeruginosa, Salmonella bongori, Salmonella cholerasuis, Salmonella enterica, Salmonella paratyphi, Salmonella typhi, Salmonella typhimurium, Moxarella catarrhalis, Shigella boydii, Shigella dysenteriae, Shigella flexneri, Shigella sonnei, Treponema pallidum, Yersinia enterocolitica, Yersinia pestis or any species falling within the genera of any of the above species.

15. A candidate antibiotic compound identified using the method of claim 1.

16. A method for identifying a gene which is required for proliferation of a cell comprising:
(a) contacting a cell with an antisense nucleic acid flanked on each end by at least one stem-loop structure,
(b) determining whether said antisense nucleic acid inhibits proliferation of said cell; and
(c) identifying the gene in said cell which encodes the mRNA which is complementary to said antisense nucleic acid or a portion thereof.

17. The method of claim 16, wherein said step of determining whether said antisense nucleic acid inhibits the proliferation of said cell comprises comparing the proliferation of said cell transcribing a first level of said antisense nucleic acid to the proliferation of said cell which transcribes a lower level of said antisense nucleic acid or which does not transcribe said antisense nucleic acid.

18. The method of claim 16, wherein said at least one stem-loop structure formed at the 5′ end of said antisense nucleic acid comprises a flush, double stranded 5′ end.

19. The method of claim 16, wherein the activity of at least one enzyme involved in RNA degradation has been reduced in said cell.

20. The method of claim 19, wherein said at least one enzyme involved in RNA degradation is selected from the group consisting of RNase E, RNase II, RNase III, polynucleotide phosphorylase, and poly(A) polymerase.

21. The method of claim 16, wherein said antisense nucleic acid comprises a random genomic fragment from said organism.

22. The method of claim 16, wherein said step of contacting said cell with said antisense nucleic acid comprises transcribing said antisense nucleic acid from a promoter.

23. The method of claim 22, wherein said promoter is regulatable.

24. The method of claim 22, wherein the first transcribed nucleotide from said promoter is the first nucleotide of a 5′ stem-loop structure.

25. The method of claim 16, wherein said at least one stem-loop structure comprises SEQ ID NO.: 5.

26. The method of claim 16, wherein said antisense nucleic acid lacks RNase E recognition sites.

27. The method of claim 16, wherein said at least one stem-loop structure lacks RNase III recognition sites.

28. The method of claim 16, wherein said at least one stem-loop structure lacks a ribosome binding site.

29. The method of claim 16, wherein said at least one stem-loop structure formed at the 3′ end of said antisense nucleic acid comprises at least one rho independent terminator.

30. The method of claim 16, wherein said cell is a gram-negative bacterium.

31. The method of claim 16, wherein said sensitized cells are selected from a group consisting of Bacteroides fragilis, Bordetella pertussis, Burkholderia cepacia, Campylobacter jejuni, Chlamydia pneumoniae, Chlamydia trachomatus, Enterobacter cloacae, Escherichia coli, Haemophilus influenzae, Helicobacter pylori, Klebsiella pneumoniae, Neisseria gonorrhoeae, Neisseria meningitidis, Pasteurella haemolytica, Pasteurella multocida, Proteus vulgaris, Pseudomonas aeruginosa, Salmonella bongori, Salmonella cholerasuis, Salmonella enterica, Salmonella paratyphi, Salmonella typhi, Salmonella typhimurium, Moxarella catarrhalis, Shigella boydii, Shigella dysenteriae, Shigella flexneri, Shigella sonnei, Treponema pallidum, Yersinia enterocolitica, Yersinia pestis or any species falling within the genera of any of the above species.

32. A method for manufacturing an antibiotic comprising the steps of:
(a) contacting sensitized cells which express a sublethal level of an antisense nucleic acid flanked on each end by at least one stem-loop structure with a compound;
(b) identifying a compound which substantially inhibits the proliferation of said sensitized cells relative to cells which have not been sensitized; and
(c) manufacturing the compound so identified.

33. The method of claim 32, wherein said at least one stem-loop structure formed at the 5′ end of said antisense nucleic acid comprises a flush, double stranded 5′ end.

34. The method of claim 32, wherein the activity of at least one enzyme involved in RNA degradation has been reduced in said sensitized cells.

35. The method of claim 32, wherein said antisense nucleic acid comprises a random genomic fragment from said sensitized cells.

36. The method of claim 32, wherein said sensitized cells comprise a gram-negative bacterium.

37. The method of claim 32, wherein said sensitized cells are selected from a group consisting of Bacteroides fragilis, Bordetella pertussis, Burkholderia cepacia, Campylobacter jejuni, Chlamydia pneumoniae, Chlamydia trachomatus, Enterobacter cloacae, Escherichia coli, Haemophilus influenzae, Helicobacter pylori, Klebsiella pneumoniae, Neisseria gonorrhoeae, Neisseria meningitidis, Pasteurella haemolytica, Pasteurella multocida, Proteus vulgaris, Pseudomonas aeruginosa, Salmonella bongori, Salmonella cholerasuis, Salmonella enterica, Salmonella paratyphi, Salmonella typhi, Salmonella typhimurium, Moxarella catarrhalis, Shigella boydii, Shigella dysenteriae, Shigella flexneri, Shigella sonnei, Treponema pallidum, Yersinia enterocolitica, Yersinia pestis or any species falling within the genera of any of the above species.


SEQUENCE LISTING (7 sequences)

SEQ ID NO: 1 - DNA, 32 bp, Artificial Sequence
  Primer
ccggaagcttataaaacgaaaggctcagtcga

SEQ ID NO: 2 - DNA, 20 bp, Artificial Sequence
  Primer
aggtgcctcactgattaagc

SEQ ID NO: 3 - DNA, 33 bp, Artificial Sequence
  Oligonucleotide
aattgtgagcggatcacaattgaattcccggga

SEQ ID NO: 4 - DNA, 33 bp, Artificial Sequence
  Oligonucleotide
agcttcccgggaattcaattgtgatccgctcac

SEQ ID NO: 5 - RNA, 21 nt, Artificial Sequence
  Stem Loop Structure
aauugugagcggaucacaauu

SEQ ID NO: 6 - DNA, 21 bp, Artificial Sequence
  LexU1 Primer
gtgagcggataacaatgatac

SEQ ID NO: 7 - DNA, 20 bp, Artificial Sequence
  ClaU1 Primer
aggtgcctcactgattaagc